(19) United States
(12) Patent Application Publication
(10) Pub. No.: US 2023/0277071 A1
(43) Pub. Date: Sep. 7, 2023

(54) SYSTEMS, METHODS, AND DEVICES FOR NON-INVASIVE AND CONTINUOUS HEMODYNAMIC MEASUREMENT

(71) Applicant: MacDonald, Dettwiler and Associates Inc., Brampton (CA)

(72) Inventors: Yannick D′Mello, Montreal (CA); Michel Arthur Lortie, Saint-Lazare (CA); David V. Plant, Westmount (CA); James Skoric, Montreal (CA)

(21) Appl. No.: 18/007,632

(22) PCT Filed: Jun. 1, 2021
(86) PCT No.: PCT/CA2021/050741

§ 371 Date: Dec. 1, 2022

Related U.S. Application Data
(60) Provisional application No. 63/033,103, filed on Jun. 1, 2020.

(51) Int. Cl. A61B 5/021

(52) U.S. Cl. CPC A61B 5/021; A61B 2562/0219

(57) ABSTRACT

Provided is a system, method, and device for non-invasive hemodynamic measurement of a subject. The method includes identifying vibrational pulses V1 and V2 and vibrations corresponding to cardiac mechanical motion from vibrational cardiography (VCG) data, the VCG data derived from a vibration signal acquired at the surface of the chest of the subject corresponding to cardiac-induced vibrations; determining a vibration feature from the vibration signal; and determining a hemodynamic measurement from the vibration feature.

### TECHNICAL FIELD

The following relates generally to vital sign measurement technology, and more particularly to systems, methods and devices for non-invasive continuous hemodynamic measurement.

### INTRODUCTION

The measurement of vital signs, such as blood pressure, is critical to understanding and determining the status of the body’s vital or life-sustaining functions. Vital sign measurement can be used to help assess the general physical health of a person, give indications as to the possible existence of diseases, and show progress towards recovery. For example, regular cardiac monitoring can facilitate the diagnosis, analysis, and prevention of cardiac ailments. Continuous monitoring of vital signs provides an opportunity to detect irregular and anomalous activity at an early stage, which can then inform subsequent prevention and treatment strategies.

Blood pressure is the pressure of circulating blood on the walls of the blood vessels. The term “blood pressure” usually refers to the pressure in large arteries of the systemic circulation. Blood pressure is usually expressed in terms of the systolic pressure (maximum during one heartbeat) over diastolic pressure (minimum in between two heartbeats) and is measured in millimeters of mercury (mmHg), above the surrounding atmospheric pressure. Normal resting blood pressure in an adult is approximately 120 millimetres of mercury (16 kPa) systolic, and 80 millimetres of mercury (11 kPa) diastolic, abbreviated as “120/80 mmHg”. Deviations from normal resting blood pressure values can be indicative of health issues such as cardiac ailments and cardiovascular disease.

Existing approaches to blood pressure measurement include invasive and non-invasive techniques. Catheterization is an invasive measurement technique that represents the gold standard in blood pressure measurement. This method measures instantaneous blood pressure by placing a strain gauge in fluid contact with blood at any arterial site (e.g., radial artery, aorta). However, catheterization is a highly invasive technique and is usually restricted to hospital settings.

Non-invasive blood pressure measurement techniques include cuff-based and cuffless techniques. Examples of cuff-based techniques include auscultation, oscillometry, and volume clamping. Such cuff-based techniques use multiple pieces of equipment and can require certain professional skill to perform accurately. Further, while cuff-based techniques are generally useful for discrete measurements of an individual’s blood pressure, they are generally not suitable options for continuous blood pressure measurement as they are not only obtrusive but also invasive to the patient because inflation of the cuff results in the crushing of blood vessels carrying blood in the arm and slowly releasing pressure as points at which crushed blood vessels bounce back are monitored. Additionally, cuff-based techniques do not truly provide a continuous blood pressure measurement but rather blood pressure measurements at discrete points in time.

Pulse-transit time (PTT) is a cuffless non-invasive blood pressure measurement technique. PTT systems determine pulse transit time, which is the time it takes for a pulse to propagate from a proximal point to a distal point in the arterial tree. PTT has been shown to be physiologically related to BP through pulse wave propagation models. PTT systems use two pieces of equipment (e.g. finger sensor and chest sensor) that need to be coordinated and a third piece of equipment for performing the coordination. Existing PTT setups can be cumbersome and are more suitable for use in special cases. Further, the equipment setup makes PTT unsuitable for continuous monitoring in remote monitoring or telehealth situations because undressing and putting the equipment on adds unwanted inconvenience. Some PTT systems even require infrequent cuff use for calibration measurement.

Existing approaches to blood pressure measurement are not suitable for continuous remote health monitoring applications as they can be invasive, involve complicated equipment setups that add inconvenience and reduce freedom to move, and may require a second person to administer the measurement or process the results. Such disadvantages of existing measurement systems limit their practicality for remote applications such as telehealth monitoring, military applications including monitoring vital signs of wounded soldiers in transit from battle field to a definitive care setting, and spaceflight missions where health professionals may not be physically present or accessible and where maintaining health states can be particularly critical.

Accordingly, there is a need for improved systems and methods for hemodynamic measurement that overcome at least some of the disadvantages of existing systems and methods.

### SUMMARY

A method of non-invasive hemodynamic measurement of a subject is provided. The method includes identifying vibrational pulses V1 and V2 from vibrational cardiography (VCG) data, the VCG data derived from a vibration signal acquired at the surface of the chest of the subject corresponding to cardiac-induced vibrations. The method further includes determining a vibration feature from the vibration pulses V1 and V2. The method further includes determining a hemodynamic measurement from the vibration feature.

The hemodynamic measurement may be blood pressure.

The method may further include identifying, extracting, or analyzing a respiration signal from the VCG data. The respiration signal may be analyzed without extracting the respiration signal from the VCG data.

The method may further include identifying or analyzing individual cardiac cycles in the VCG data.

Determining the blood pressure measurement may include determining maxima, minima, or mean of a central aortic or left ventricular pressure waveform for each cardiac cycle in real-time.

The vibration signal may include a linear acceleration component and a rotational velocity component.

The vibration signal may include six orthogonal motion signals.

Determining the vibration feature may include quantifying the fraction of energy of stroke volume converted to vibration. The energy may be kinetic energy.

The vibration feature may be determined using a linear acceleration component of the vibration signal and a rotational velocity component of the vibration signal.

Determining the vibration feature may include determining any one or more of jerk, amplitude, frequency, phase, and a cardiac time interval from a linear acceleration component or rotational velocity component of the vibration signal.

The method may further include filtering or demodulating any one or more of motion artifact, sensor placement, exertion, respiration, and a physical characteristic of the subject from the vibration signal.

The method may further include extracting or analyzing the vibrational pulses V1 and V2 from the VCG data.

The hemodynamic measurement may be a blood pressure measurement.

A system for non-invasive blood pressure measurement of a subject is also provided. The system includes a sensor device including an accelerometer and a gyroscope. The sensor device detects vibrations at the surface of the chest of the subject corresponding to cardiac mechanical activity of the heart and transmits a vibration signal associated with the detected vibrations. The system also includes a computing device communicatively connected to the sensor device via a data communication link. The computing device includes: a communication interface for receiving the vibration signal from the sensor device via the data communication link; a processor configured to determine a vibration feature from the vibration signal, determine a blood pressure measurement from the vibration feature, and generate a human-readable format of the blood pressure measurement; a memory for storing the blood pressure measurement; and a display device for outputting the blood pressure measurement in the human-readable format.

The processor may be further configured to identify vibrational pulses V1 and V2 from vibrational cardiography (VCG) data, wherein the VCG data is derived from the vibration signal, and determine the vibration feature from the vibrational pulses V1 and V2.

The processor may be further configured to identify, extract, or analyze a respiration signal from the VCG data. The processor may analyze the respiration signal without extracting the respiration signal from the VCG data.

The processor may be further configured to identify or analyze individual cardiac cycles in the VCG data.

Determining the blood pressure measurement by the processor may include determining maxima, minima, or mean of a central aortic or left ventricular pressure waveform for each cardiac cycle in real-time.

The vibration signal may include a linear acceleration component and a rotational velocity component.

The vibration signal may include six orthogonal motion signals.

Determining the vibration feature by the processor may include quantifying the fraction of energy of stroke volume converted to vibration. The energy may be kinetic energy.

Determining the vibration feature by the processor may include determining any one or more of jerk, amplitude, frequency, phase, and a cardiac time interval from a linear acceleration component or rotational velocity component of the vibration signal.

The processor may be further configured to filter or demodulate any one or more of motion artifact, sensor placement, exertion, respiration, or a physical characteristic from the vibration signal.

The processor may be further configured to extract or analyze the vibrational pulses V1 and V2 from the VCG data.

A computer system for non-invasive blood pressure measurement of a subject is also provided. The computer system includes a communication interface for receiving a vibration signal. The vibration signal is detected at the surface of the chest of the subject and corresponds to cardiac mechanical activity of the heart. The computer system further includes a processor configured to: generate vibrational cardiography (VCG) waveform data from the vibration signal; filter and demodulate the VCG waveform data to generate a processed VCG waveform; determine a vibration feature from the processed VCG waveform data; determine a blood pressure measurement from the vibration feature; and generate a human-readable format of the blood pressure measurement. The computer system further includes a display device for outputting the blood pressure measurement in the human-readable format.

The processor may be further configured to identify vibrational pulses V1 and V2 from the processed vibrational cardiography waveform data and determine the vibration feature from the vibrational pulses V1 and V2.

The filtering and demodulating by the processor may include extracting or analyzing a respiration signal from the VCG waveform data. The respiration signal may be analyzed without extracting the respiration signal from the VCG data. For example, the processor may use machine learning techniques to analyze the respiration signal without extracting the respiration signal from the VCG data.

The processor may be further configured to identify individual cardiac cycles in the processed VCG waveform data.

Determining the blood pressure measurement from the vibration feature by the processor may include determining maxima, minima, or mean of a central aortic or left ventricular pressure waveform for each cardiac cycle in real-time.

The vibration signal may include a linear acceleration component and a rotational velocity component.

The vibration signal may include six orthogonal motion signals.

Determining the vibration feature from the processed VCG waveform data by the processor may include quantifying the fraction of energy of stroke volume converted to vibration. The energy may be kinetic energy.

Determining the vibration feature from the processed VCG waveform data by the processor may include determining any one or more of jerk, amplitude, frequency, phase, and a cardiac time interval from a linear acceleration component or rotational velocity component of the vibration signal.

The filtering and demodulating by the processor may include filtering or demodulating any one or more of motion artifact, sensor placement, exertion, respiration, and a physical characteristic of the subject from the vibration signal.

The processor may be further configured to extract or analyze the vibrational pulses V1 and V2 from the processed VCG waveform data.

In another aspect, a method of non-invasive hemodynamic measurement of a subject is provided. The method includes identifying cardiac-induced vibrations from vibrational cardiography (VCG) data. The VCG data is derived from a vibration signal acquired at the surface of the chest of the subject corresponding to the cardiac-induced vibrations. The method further includes determining a vibration feature from the vibration signal and determining a hemodynamic measurement from the vibration feature.

The hemodynamic measurement may be a blood pressure measurement.

The cardiac-induced vibrations may include vibrational pulses V1 and V2. The vibrational pulses V1 and V2 may correspond to the primary heart sounds. The vibration feature may or may not be directly related to vibrational pulses V1 and V2. The cardiac-induced vibrations may include vibrations corresponding to cardiac mechanical motion.

The cardiac-induced vibrations may be vibrations having a frequency less than 20 Hz. The cardiac-induced vibrations may be vibrations having a frequency in the infrasonic range. The cardiac-induced vibrations may be vibrations having a frequency in the 1 Hz to 2 Hz range.

In another aspect, a computer system for non-invasive hemodynamic measurement of a subject is provided. The computer system comprises a processor and a memory in communication with processor. The memory stores computer executable instructions which when executed by the processor cause the computer system to: identify cardiac-induced vibrations from vibrational cardiography (VCG) data, the VCG data derived from a vibration signal acquired at the surface of the chest of the subject corresponding to the cardiac-induced vibrations; determine a vibration feature from the vibration signal; and determine a hemodynamic measurement from the vibration feature.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a schematic representation of the heart;

FIG. 2 is a flow diagram of a method of non-invasive continuous blood pressure measurement, according to an embodiment;

FIG. 3 is a flow diagram of a method of non-invasive continuous blood pressure measurement using vibrational cardiography (VCG), according to an embodiment;

FIG. 4 is a block diagram of a system for non-invasive continuous blood pressure measurement using a wearable sensor module, according to an embodiment;

FIG. 5 is a block diagram of a computer system for performing non-invasive continuous blood pressure measurement, according to an embodiment;

FIG. 6 is a flow diagram of a method of non-invasive continuous blood pressure measurement using the system of FIG. 4, according to an embodiment;

FIG. 7 is a block diagram of a computing device of the system of FIG. 4;

FIG. 8 is a diagram illustrating biometric measurements that may be determined from a VCG signal using the systems of the present disclosure, such as the system of FIG. 4;

FIG. 9 is an example seismocardiography (SCG) waveform showing valve actions;

FIG. 10 is an example electrocardiography (ECG) waveform;

FIG. 11 is a schematic representation of the blood circulation circuit for a cardio-vascular system;

FIG. 12 is a cardiac cycle diagram (or Wiggers diagram) presenting blood pressure in relation to the physical movements of the heart and its electrical commands;

FIG. 13 is a block diagram illustrating an analytical approach to determining aortic blood pressure from vibration measurements or features at the xiphoid process implemented by the systems of the present disclosure, according to an embodiment;

FIG. 14 is a block diagram illustrating a machine learning-based approach to determining aortic blood pressure from vibrational measurements or features at the xiphoid process, according to an embodiment;

FIG. 15 is a diagram illustrating equipment used for physiological measurements by the non-invasive physiological activity monitoring system, according to an embodiment;

FIG. 16 is a diagram illustrating equipment used for physiological measurements in a non-invasive physiological activity monitoring system laboratory, according to an embodiment;

FIG. 17 is a system configuration diagram for a non-invasive physiological activity monitoring system of the present disclosure, according to an embodiment;

FIG. 18 is a system configuration diagram for a non-invasive physiological activity monitoring system of the present disclosure, according to another embodiment;

FIG. 19 is a histogram of a sampling rate of a system of the present disclosure using a sequential method, according to an embodiment;

FIG. 20 is a histogram of a sampling rate of a system of the present disclosure using a multi-threading method, according to an embodiment;

FIG. 21 is an example web-based interface for a system of the present disclosure, according to an embodiment;

FIG. 22 is a schematic representation showing a sensor set-up and experimental procedure for experimental work related to the system of the present disclosure;

FIG. 23A is a sensor placement grid;

FIG. 23B is an image illustrating the sensor positions of FIG. 16A traced on the chest of a subject;

FIG. 24 is a graph illustrating AO amplitude as a function of sensor position and R-AO delay as a function of sensor position (change in AO position and timing);

FIG. 25 is a graph illustrating wave form changes due to (a) high lung volume, (b) low lung volume, (c) across all subjects, and (d) RMS for all subjects;

FIG. 26 is a schematic diagram of the human heart indicating valves, ventricles, atria, and major blood vessels;

FIG. 27 is a graph illustrating the cardiac pressure cycle showing (a) typical changes in ventricular pressure and volume, (b) the P-V loop representing a cardiac cycle, and (c) Wiggers diagram displaying synchronized changes in pressure, volume, ECG, PCG, and SCG;

FIG. 28 is a spectral profile of a VCG signal for the az, gx, and gy axes (three main axes);

FIG. 29 is a graph illustrating respiration volume integrated directly from the spirometer (yellow), with resets (red), and calculated from the IMU sensor (blue);

FIG. 30 is a graph illustrating (a) a comparison between the outputs of the VarWin (top) and DerWin (bottom) functions and (b) the DerWin output separated by cardiac cycles;

FIG. 31 is a graph illustrating (a) acceleration measured by the IMU compared with twice-differentiated displacement from the Keyence sensor, (b) integrated acceleration from the IMU compared with differentiated displacement from the Keyence sensor, (c) twice-integrated acceleration from the IMU compared with the displacement measured by the Keyence sensor, and (d) The velocity-squared term of the vibrational Kinetic Energy detected by the (blue) IMU accelerometer, (red) IMU gyroscope, and (yellow) laser displacement sensor;

FIG. 32 is a graph illustrating processed VCG signal for the different physiological metrics, including (a) NIBP and VCG waveforms, (b) RV derived from the spirometer and the IMU, (c) HR, BTB, and LVET calculations from the SCG, ECG, ICG, and NIBP signals, (d) Central aortic pressure waveforms fitted to the sBP and dBP measurements obtained from the NIBP during the systolic phase of each cardiac cycle, and (e) Calibrated pressure obtained by simply scaling the amplitudes of the SCG signal to match the first ten seconds of data;

FIG. 33 is a three-dimensional representation of the geometry of a proposed model to study electrical activity at the heart;

FIG. 34 is a graph illustrating a change in pressure caused by a potential difference at the ventricle;

FIG. 35 is a three-dimensional representation of the geometry of a proposed valve model;

FIG. 36 is a graph illustrating pressure differential at the input and the output of the valve;

FIG. 37 is a graph illustrating (a) deformation of the heart valve at 0.07s (at the beginning when input pressure is higher than the output pressure), and (b) deformation at 0.21s (when pressure differential between the input and the output is maximum);

FIG. 38 is a graph illustrating (a) simulated acceleration at the XP compared to the (b) acceleration acquired through experiment;

FIG. 39 is a three-dimensional representation of the geometry of a wave propagation model;

FIG. 40 is a graph illustrating correlation (left) and Bland-Altmann (right) plots of the measured systolic blood pressure in comparison with the calculated VarWin amplitude at the AO event for each subject (excluding calibration measurement);

FIG. 41 is a graph illustrating correlation (left) and Bland-Altmann (right) plots of the measured diastolic blood pressure in comparison with the calculated VarWin amplitude at the AC event for each subject (excluding calibration measurement);

FIG. 42 is a graph illustrating correlation plots of the 1D CNN predictions for systolic and diastolic BP;

FIG. 43 is a schematic representation of (a) general placement of the ICG electrodes (green), ECG electrodes (blue), and VCG sensor (red), and (b) system configuration enabling simultaneous recordings of ECG, ICG, and VCG;

FIG. 44 is a flow diagram of a method of signal processing steps for obtaining vibrational pulses from an acquired vibrational motion signal, according to an embodiment;

FIG. 45 is a graph illustrating simultaneous recordings of (a) ECG with circles representing the identified R-peaks; (b) Raw (blue) and filtered (red) ICG with the annotated B- and X-points shown as circles and crosses respectively; and (c) SCG acceleration αZ (blue) and jerk magnitude |daZ/dt| (red), (d) X-axis GCG gX (blue) and its RKE component gX2 (red), and (e) gY (blue) and gY2 (red) with dotted, black lines representing the identified timestamps of V1 and V2;

FIG. 46 is a graph illustrating correlation of heart rate calculated from (a) VCG and (b) ICG with a r2 of 0.9887 and 0.9824 respectively, when referenced with ECG;

FIG. 47 is a graph illustrating correlation of (a) the time interval from the ECG R-peak to both V2 from VCG and B from ICG, and (b) LVETF obtained from VCG and ICG with a r2 of 0.251 and 0.2797 respectively;

FIG. 48 is a diagram illustrating (a) Placement of the inertial measurement unit (IMU) on the xiphoid process of the sternum (shown in black) with its orientation represented by the Cartesian reference axis, and the electrocardiography (ECG) electrodes (shown in green) attached to the torso. The corresponding signal morphology of a single CC is shown for (b) acceleration in all axis components and (c) gyration in all axis components.

FIG. 49 is a block diagram illustrating an overall architecture of a proposed CNN to classify lung volume state of VCG cardiac cycles, according to an embodiment;

FIG. 50 is a diagram illustrating system configuration for a system of the present disclosure including an RPI and IMU, according to an embodiment;

FIG. 51 is a diagram illustrating (a) sensor and electrode placement. (b) Z-axis acceleration. (c) X-axis;

FIG. 52 is a graph illustrating correlation and Bland Altman plots comparing VCG-derived HR to ECG-derived HR from across the entire dataset;

FIG. 53 is a graph illustrating ensemble averages for a single subject when (a) supine, (b) facing left, (c) facing right, (d) sitting, and (e) standing;

FIG. 54 is a diagram illustrating (a) Spirometer (red) and IMU (black) placement with corresponding acceleration coordinates and (b) Experimental dataflow diagram;

FIG. 55 is a graph illustrating a) raw x-axis acceleration (red) and y-axis gyration (blue), (b) Savitsky-Golay filtered x-axis acceleration (red) and y-axis gyration (blue), and (c) reference lung volume (All plots were normalized);

FIG. 56 is a schematic representation of blood flow from the left ventricle to the finger artery and corresponding vibrational activity associated with cardiac mechanical activity of the blood flow, which can be leveraged by the systems and methods for hemodynamic measurement of the present disclosure;

FIG. 57A is a graphical representation of an ECG waveform, aortic pressure waveform, and SCG waveform over time including a pre-ejection period (PEP) and left ventricular ejection time (LVET);

FIG. 57B is a graphical representation showing graphs of linear displacement and angular displacement, and vector norms of three axes of linear displacement and angular displacement, illustrating a relationship between displacement (from vibration signal, VCG) and cardiac pressure;

FIG. 58A is a first graph illustrating an ECG waveform and pressure waveforms for aortic pressure, left ventricular pressure, pulmonary artery pressure, and right ventricular pressure, and a second graph illustrating velocity plotted against time for the left atrium, left ventricle, right atrium, right ventricle, and sinoatrial node derived from a cardiac model of the circulatory system showing correspondence with the first graph;

FIG. 58B is a schematic representation of the cardiac system and a schematic representation of a cardiac model of the cardiac system achieved mechanically and used to prove connection between vibrations and cardiac pressure, the cardiac model used to generate the second graph of FIG. 58A; and

FIG. 59A is a graphical representation of a transfer function associated with cardiac pressure change and a graph illustrating evolution of pressure waveform from aorta to finger; and

FIG. 59B is a graph comparing blood pressure against time for a finger-based measurement and an aorta estimate.

### DETAILED DESCRIPTION

Various apparatuses or processes will be described below to provide an example of each claimed embodiment. No embodiment described below limits any claimed embodiment and any claimed embodiment may cover processes or apparatuses that differ from those described below. The claimed embodiments are not limited to apparatuses or processes having all of the features of any one apparatus or process described below or to features common to multiple or all of the apparatuses described below.

One or more systems described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, and personal computer, cloud-based program or system, laptop, personal data assistance, cellular telephone, smartphone, or tablet device.

Each program is preferably implemented in a high-level procedural or object oriented programming and/or scripting language to communicate with a computer system. However, the programs can be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein.

A description of an embodiment with several components in communication with each other does not imply that all such components are required. On the contrary, a variety of optional components are described to illustrate the wide variety of possible embodiments of the present invention.

Further, although process steps, method steps, algorithms or the like may be described (in the disclosure and / or in the claims) in a sequential order, such processes, methods and algorithms may be configured to work in alternate orders. In other words, any sequence or order of steps that may be described does not necessarily indicate a requirement that the steps be performed in that order. The steps of processes described herein may be performed in any order that is practical. Further, some steps may be performed simultaneously.

When a single device or article is described herein, it will be readily apparent that more than one device / article (whether or not they cooperate) may be used in place of a single device / article. Similarly, where more than one device or article is described herein (whether or not they cooperate), it will be readily apparent that a single device / article may be used in place of the more than one device or article.

The following relates generally to vital sign measurement, and more particularly to systems, methods, and devices for non-invasive hemodynamic measurement. In a particular embodiment, the hemodynamic measurement is a blood pressure measurement. The blood pressure measurement may be a continuous blood pressure measurement. While the terms “blood pressure”, “blood pressure measurement”, or the like may be referred to in the present disclosure, it is to be understood that variations of the systems and methods of the present disclosure may be similarly used to determine a hemodynamic measurement (of which blood pressure is one example).

The present disclosure provides systems and methods for blood pressure measurement which can determine continuous central aortic blood pressure of a subject by analyzing vibrational cardiography (VCG) signals. VCG data can be correlated with mechanical cardiac functions, including by measuring and analyzing myocardial vibrations generated by cardiac activity (corresponding to cardiac phase transitions and primary heart sounds), which vibrations are detected at the sternum as linear acceleration and rotational velocity. The systems and methods may, in effect, model cardiac mechanical motion of heart components by detecting vibrations at the surface of the chest.

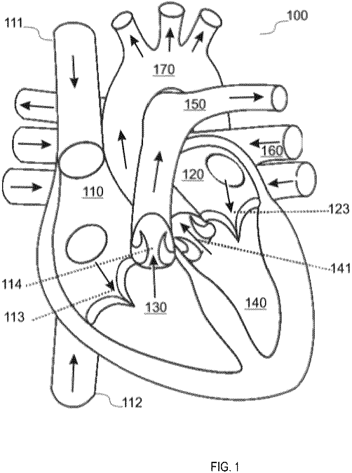

As described herein, the present disclosure provides systems and methods for blood pressure measurement which process and analyze VCG signals detected at the surface of the chest and which correspond with mechanical motions of the heart (i.e. cardiac-induced vibrations). To further highlight and illustrate the principle of operation of the systems and methods described herein, various motions, functions, and components of the human heart will now be described with reference to FIG. 1.

The heart, shown generally at 100, includes four chambers, two atriums 110, 120 and two ventricles 130, 140, forming a notional four chamber pump divided into two portions, divided by sides. The four chambers of the heart 110, 120, 130, 140 are coupled by valves which open and close in response to pressure differentials generated across the two sides of the valve. The pressure differential is generated by contraction and relaxation of heart components, which contraction occurs in response to electrical stimuli.

The heart 100 pumps blood through the circulatory system of the body, pumping oxygenated blood to the body’s organs and cells and deoxygenated blood to the lungs. The pumping action is derived from the rhythmic contraction and relaxation of the heart muscle.

The right side of the heart 100, comprising the right atrium 110 and the right ventricle 130, receives deoxygenated blood from the systemic circulatory system via the superior vena cava 111 and the inferior vena cava 112. The right atrium 110 fills and once filled the right tricuspid valve 113 opens to allow blood to flow into and to fill the right ventricle 130. Upon contraction of the right ventricle 130, blood is ejected through the pulmonary semilunar valve 114 into the pulmonary artery 150 toward the lungs (not shown) for oxygenation.

The left side of the heart 100, comprising the left atrium 120 and the left ventricle 140, receives oxygenated blood returning from the lungs via the pulmonary vein 160. At the end of a cardiac cycle, the left atrium 120 is relaxed and fills with blood due to venous return. The pressure in the left ventricle 140 decreases as the chamber distends. The mitral valve 123 opens once the left atrium pressure exceeds the left ventricular pressure. Opening of the mitral valve 123 allows blood to flow into and fill the left ventricle 140. Upon contraction of the left ventricle 140 (closing of the mitral valve 123 and opening of aortic valve 141), oxygenated blood is ejected through the aortic valve 141 into the aorta 170 and into the rest of the body.

The physical events that occur in the operation of the heart 100, as described above, are characterized by vibration and/or displacement events in the chest cavity resulting from such events. These vibrations are consistently present, though the frequency or intensity of such vibrations may vary due to factors such as physical exertion level. These vibrations travel from the heart 100 through the thoracic cavity and manifest on the surface of the chest, where they can be detected using sensor technology.

A section of particular interest for the present disclosure is the left side of the heart 100 including the left atrium (120), the left ventricle (140), mitral valve 123, and aortic valve 141.

Research indicates that the vibrations detected by the VCG related to the first primary heart sound are caused by the closure of the atrioventricular valves (e.g. mitral valve 123).

The first heart sound is generated when sudden closure of the mitral valve 123 (AV valves) results in oscillation of the blood in the left ventricle 140. This oscillation causes vibrations. The left ventricle 140 compresses and ejects blood into the aorta 170. The aortic valve 141 closes as a result of a reversal of the energy gradient of blood across the aortic valve 141 induced by relaxation of the left ventricle 140 and commensurate fall in intraventricular pressure. The abrupt closure of the aortic valve 141 causes the second primary heart sound. The first primary heart sound indicates the end of the diastolic phase and start of the systolic phase of the cardiac cycle. The second heart sound indicates the end of the systolic phase and start of the diastolic phase.

While the contraction of the ventricles (left 140 and right 130) is driven by the QRS complex, which is an electrical signal, the operation of the valves 123, 141 is controlled by the pressure gradient across the valve 123, 141 which is a product of the contraction.

As there is no storage or reservoir for blood in the cardiovascular system, as oxygen demand increases (due to, for example, physical exertion), the entire system must increase its cadence which results in a combination of increased heart rate and respiration volume with consequential dynamic responses in blood pressure. A decrease in demand (i.e. body at rest) results in a decrease in heart rate and further dynamic responses in blood pressure.

Direct measurement of blood across the aortic valve 141 (i.e. in either the left ventricle 140 or aorta 170) is not practical as it is very invasive and not suitable for use outside a hospital setting. Accordingly, to provide a non-invasive blood pressure measurement, the systems and methods of the present disclosure measure vibration (including linear acceleration and rotational velocity) associated with the opening and closing of the mitral valve 123 and aortic valve 141 (on the left side of the heart) and the movement of blood resulting therefrom. The system may determine a measurement of the vibration produced by the blood flow caused by ventricular contraction using VCG and determine blood pressure as a proportional value relative to the force (acceleration) ejecting the blood into the aorta 170. These determinations made by the system include analyzing one or more features, attributes or artifacts of the vibrational signal (e.g. acceleration signal) such as amplitude, rate of change in acceleration, and event durations (e.g. LVET, BTB).

The rhythmic rise and fall of the blood pressure in the aorta 170 is related to the periodic injection of a mass of blood by the left ventricle 140, referred to as the stroke volume. The stroke volume possesses kinetic energy. Some fraction of the kinetic energy of the stroke volume is transferred to the thoracic structures supporting the heart 100 and aorta 170 as vibrations. The systems and methods of the present disclosure detect the kinetic energy manifested as vibrations using an accelerometer and a gyroscope and generate a vibrational cardiogram (including a seismocardiogram and gyrocardiogram) therefrom.

As described herein, the systems and methods of the present disclosure include the determination of central aortic blood pressure through the detection and analysis of vibrational cardiography (VCG) signals. The vibrations directly correspond to cardiac mechanical activity. Vibrational cardiography is a technique that combines seismocardigraphy (acceleration) and gyrocardiography (gyration) to describe vibrations at the surface of the chest. The linear component of VCG is detected as acceleration and called Seismocardiography (SCG). The angular component is detected as gyration and called Gyrocardiography (GCG). In particular, VCG may include the simultaneous measurement of SCG and GCG.

The vibrational pulses (V1, V2) corresponding to the two primary heart sounds are generated by the mechanical motion of cardiac valves (e.g. 123 and 141). The valves 123, 141 are hydraulically controlled by blood pressure differentials in the heart 100. The systems and methods described herein are configured to detect both vibrational pulses (V1 and V2) and use this information as the basis for calculating blood flow through the heart 100.

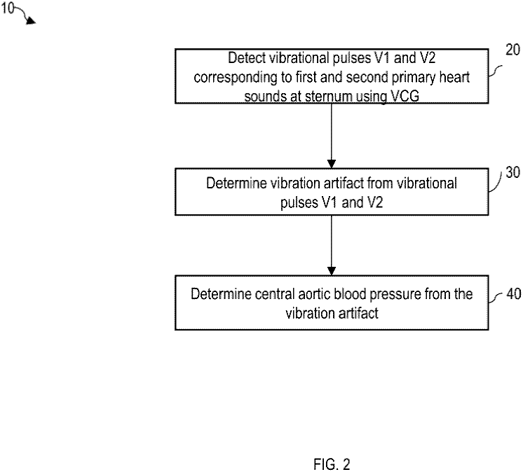

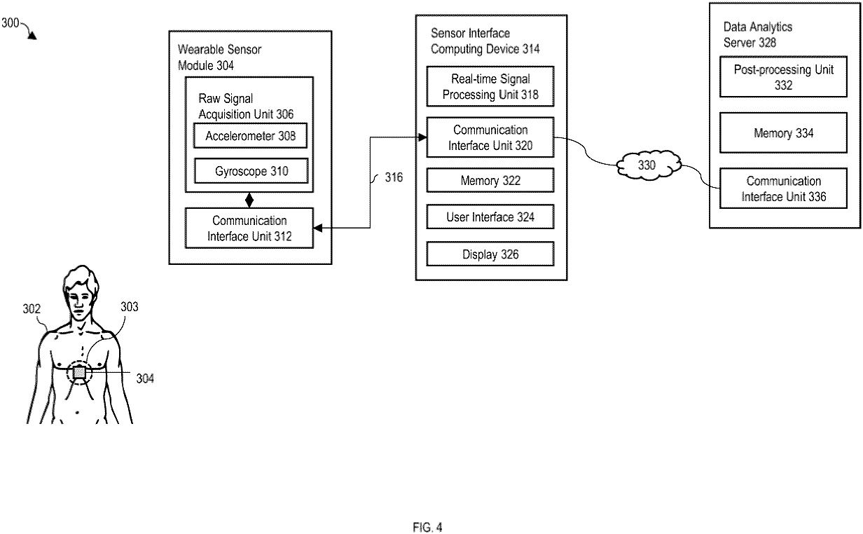

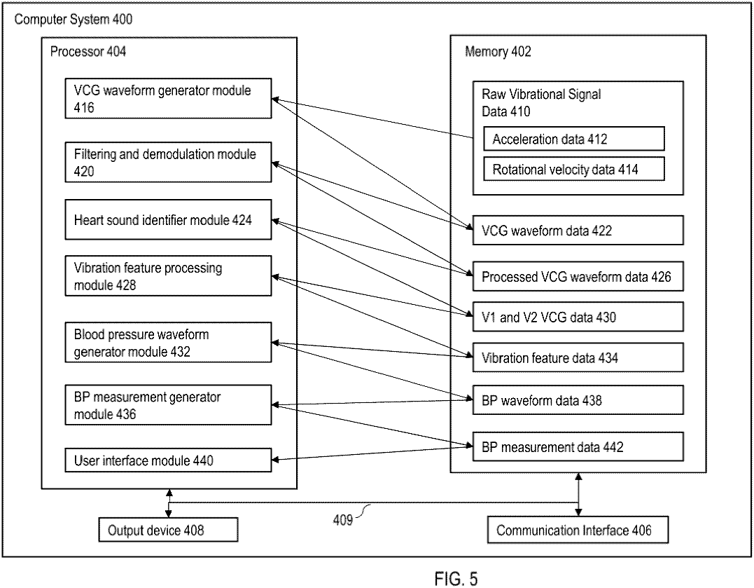

Referring now to FIG. 2, shown therein is a method 10 of non-invasive continuous blood pressure measurement, according to an embodiment. The method 10 may be implemented by one or more non-invasive blood pressure measurement systems described herein, such as systems 300 and 400 of FIGS. 4 and 5, respectively.

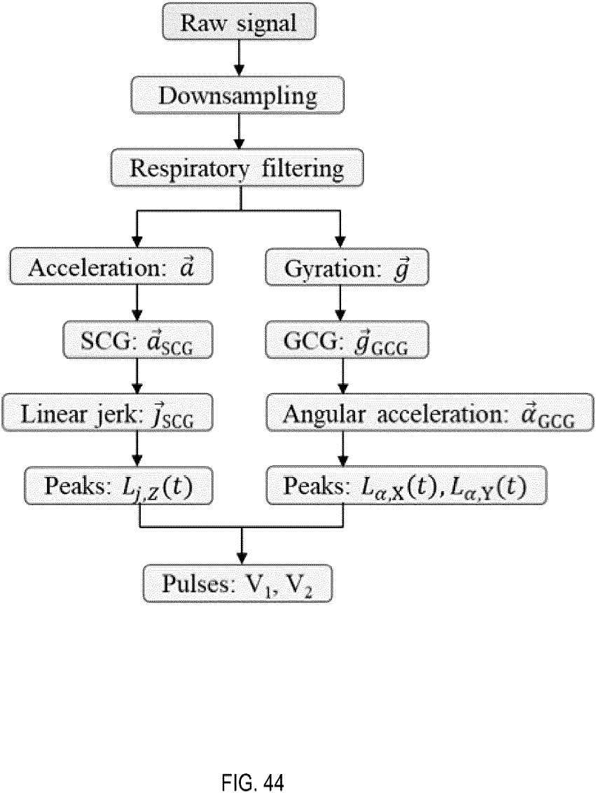

At 20, vibrations V1 and V2 corresponding with the first and second primary heart sounds are detected. V1 and V2 correspond with cardiac phase transitions. The vibrations are detected at the sternum (e.g. at the xiphoid process). The vibrations may be detected using a wearable sensor module (e.g. sensor module 304 of FIG. 4). The vibrational pulses V1 and V2 are detected using VCG, which includes a linear acceleration component (SCG) and a rotational velocity component (GCG). The detection of vibrational pulses may be performed using vibrational cardiogram transformation steps as described herein. In a particular case, vibrational pulses are detected using signal processing steps as described in reference to FIG. 44. Once the vibrational pulses are detected, information contained in the detected pulses can be processed and analyzed.

In an embodiment, step 20 may include performing vibrational cardiogram transformation steps as described herein. For example, step 20 may include performance of one or more signal processing steps illustrated in FIG. 44 (described below) and as described in reference thereto.

At 30, a vibration signal feature (vibration artifact or attribute) is determined from the V1 and V2 vibrational pulses. The vibration signal feature or vibration feature may also be referred to as a vibration artifact or vibration signal artifact. As such, the terms vibration signal feature (vibration feature) and vibration signal artifact (vibration artifact), and more generally the terms feature and artifact, may be used interchangeably throughout the present disclosure. The vibration signal feature may be considered a subcomponent of the vibration signal. Determining the vibration signal feature from the V1 and V2 vibrational pulses may include analyzing the VCG signal associated with V1 and V2. This may include modelling cardiac mechanical motions responsible for generating vibrations and hydraulic causes of motions. The vibration feature may be derived from the SCG signal, the GCG signal, or a combination. The vibration signal feature may include, for example, any one or more of amplitude, frequency, phase, rate of change in acceleration (third order derivative of position called ‘jerk’). The vibration signal feature may include a cardiac time interval (e.g. duration of blood ejection from the ventricle into the aorta called Left Ventricular Ejection Time (LVET)). The term “cardiac time interval” refers to an event duration within a cardiac cycle.

At 40, a central aortic blood pressure measurement is determined from the vibration signal feature. This may include generating a blood pressure value, such as a systolic over diastolic reading, or a blood pressure waveform, or other hemodynamic measurement.

The method 10 may be performed continuously to derive a continuous blood pressure measurement for the subject.

The method 10 is advantageously non-invasive as step 20 can be performed by applying a sensor device configured to detect vibrations at the surface of the chest.

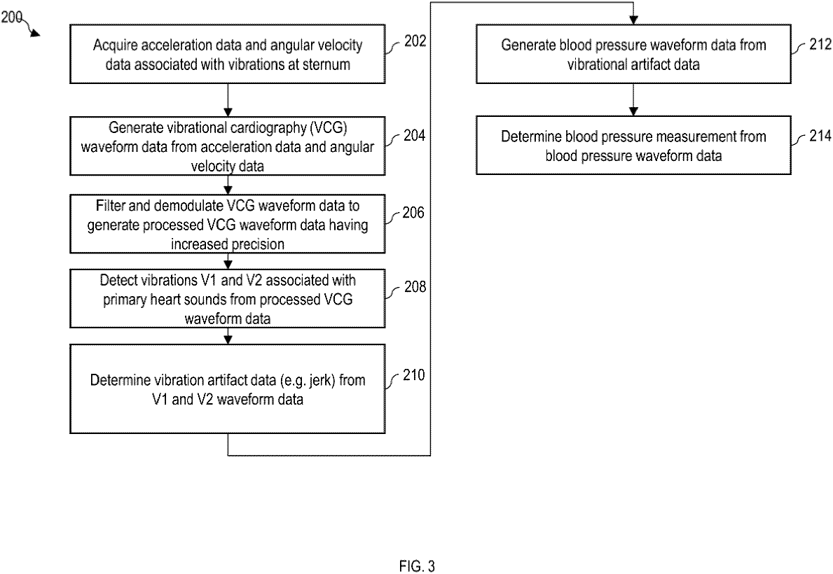

Referring now to FIG. 3, shown therein is a method 200 of non-invasive continuous blood pressure measurement using vibrational cardiography (VCG), according to an embodiment. The method 200 may be implemented by a computing device such as devices 314 and 400 of FIGS. 4 and 5, respectively, described below.

At 202, a vibration signal including a linear acceleration signal and an angular or rotational velocity signal is acquired. This vibration signal is acquired using a sensor device positioned at the sternum (xiphoid process) of a subject. The acquired vibration signal is detected at the skin and is a product of thoracic vibrations caused by cardiac mechanical activity, such as described in reference to FIG. 1.

At 204, vibrational cardiography (VCG) waveform data is generated from the acceleration signal and the rotational velocity signal. This may include sampling received signals or data.

At 206, the VCG waveform data is filtered and demodulated. This may be done to remove noise and distortions caused by external factors such as sensor placement or positioning, respiratory activity, exertion factor, etc. Filtering and demodulating the VCG waveform data generates a processed VCG waveform having an increased precision. In an embodiment, respiration effects may be filtered or demodulated from the VCG signal as described in PCT Application No. PCT/CA2018/051006, publication number WO/2020/037391, which is hereby incorporated by reference in its entirety.

At 208, the processed VCG waveform data is analyzed to identify vibrations V1 and V2 corresponding to the first and second primary heart sounds.

At 210, a vibration feature is determined from the VCG waveform data associated with V1 and V2 vibrations. This may include modelling cardiac mechanical motions responsible for generating vibrations and hydraulic causes of motions.

At 212, blood pressure waveform data is derived from the vibrational feature data.

At 214, a blood pressure measurement is determined from the blood pressure waveform data. This may include calculating or reading a particular value from the blood pressure waveform data. This step may advantageously generate a blood pressure value that is interpretable by a non-health professional.

Referring now to FIG. 4, shown therein is a system 300 for non-invasive blood pressure measurement, according to an embodiment. The system 300 is capable of implementing the methods 10 and 200 of FIGS. 2 and 3, respectively.

The system 300 integrates sensor technology and computer-implemented technology to generate continuous blood pressure measurement. The system 300 may be particularly well suited to remote monitoring applications where the subject is not in the same physical location as a medical professional. Examples of such remote monitoring applications include telehealth (patients monitoring blood pressure at home or outside the clinical setting), space travel (astronauts requiring vital sign measurement), and military combat settings such as where personnel may be injured in a first location and need transport to a proper medical facility.

The system 300 is used to determine a blood pressure measurement for a subject 302. The blood pressure measurement may be discrete or continuous.

The system 300 includes a wearable sensor module 304, a sensor interface computing device 314, and a data analytics server 328. In variations of the system 300, the data analytics server may not be included or required.

The wearable sensor module 304 is located on the surface of the skin of the subject 302 at a position on the surface of the chest near the heart. Generally, the location of the sensor module 304 on the subject 302 should be such that a sufficient vibration signal for analysis can be acquired. The specific position may be selected based on proximity to the heart, signal strength, and reducing noise (e.g. caused by propagation of the vibrational waves through the thoracic compartment or physiological activity such as respiration). In a human subject, the wearable sensor module 304 is positioned at or near the sternum. More particularly the wearable sensor module 304 may be positioned at the xiphoid process. An example target location for the sensor at the sternum of subject 302 is shown at 303. The wearable sensor module 304 may be applied to the subject 302 using any suitable adhesive means. In some cases, the adhesive means may be selected such that the sensor module 304 stays adhered to the subject 302 while in an upright position or while the subject 302 is in motion (strenuous or not).

The wearable sensor module 304 may be wireless. To that end, the wearable sensor module 304 may include wireless power and communication components. Wireless implementations of the sensor module 304 advantageously are less restrictive and complicated for the subject 302 and allow movement by the subject 302.

The wearable sensor module 304 includes a raw signal acquisition unit 306 for detecting and acquiring raw vibrational signals at the surface of the chest resulting from vibrations corresponding to the cardiac mechanical activity of the heart of the subject 302.

The raw signal acquisition unit 306 includes an accelerometer 308 for detecting a linear acceleration component of the vibration. The raw linear acceleration signal (or acceleration signal) detected by the accelerometer 308 can be used to generate VCG waveform data, namely through the generation of seismocardiography (SCG) data.

The raw signal acquisition unit 306 also includes a gyroscope 310 for detecting a rotational velocity component of the vibration. The raw rotational velocity signal (or gyration signal) detected by the gyroscope 310 can be used to generate VCG waveform data, namely through the generation of gyrocardiography (GCG) data. Collectively, the acceleration signal and rotational velocity signal may be collectively considered the “raw vibration signal” of the heart.

The raw signal acquisition may be an inertial measurement unit including at least an accelerometer and a gyroscope.

In an embodiment, the raw signal acquisition unit 306 is configured to acquire six orthogonal motion signals. The raw signal acquisition unit 306 may detect linear α^{→}SCG and rotational g^{→}GCG motion in all six orthogonal degrees of freedom (three SCG, three GCG). A more comprehensive vibration signal may be generated by integrating the six mutually orthogonal axes from the SCG and GCG (such integration may be performed by the sensor module 304 or the sensor interface computing device 314).

The wearable sensor module 304 includes a communication interface unit 312 for transmitting and receiving information to and from external devices (such as computing device 314). The communication interface unit 312 is in signal communication with the raw signal acquisition unit 306 such that information can be communication to and from the raw signal acquisition unit 306. The communication interface unit 312 receives acquired acceleration signals and rotational velocity signals from the raw signal acquisition unit 306 via the accelerometer 308 and gyroscope 310, respectively.

The wearable sensor module 304 is communicatively connected to the sensor interface computing device 314 via a communication link 316. The communication link 316 may be any suitable wired or wireless communication link for transmitting and receiving data. In an embodiment, the communication link 316 may be a short-range data communication link, such as Bluetooth.

The communication link 316 transmits raw vibration signals from the sensor module 304 to the computing device 314 and may transmit control instructions from the computing device 314 to the sensor module 306. Control instructions may include, for example, starting and stopping raw signal acquisition or signal acquisition parameters.

The sensor interface computing device 314 includes a communication interface 320 for sending and receiving data to and from external devices such as sensor module 304. The communication interface 320 receives the raw vibration signal from the wearable sensor module 304.

The sensor interface computing device 314 includes a real-time signal processing unit 318 for processing the raw vibration signal and generating a blood pressure measurement therefrom. The signal processing unit 318 is configured to perform one or more digital signal processing techniques to received vibration signals. The signal processing unit 318 may generate a continuous blood pressure measurement in real-time.

The real-time signal processing unit 318 processes the raw vibration signal, including acceleration and rotational velocity components, to generate VCG waveform data (including SCG and GCG waveform data corresponding to acceleration and rotational velocity signals, respectively). This processing may include filtering and/or demodulating VCG waveform data to remove or limit distortions caused by factors such as respiratory activity and exertion. The signal processing unit 318 may detect vibrations corresponding with cardiac phase transitions. The detected vibrations may correspond with the two primary heart sounds. The signal processing unit 318 analyzes the VCG waveform data and determines a vibration feature. The signal processing 318 uses the determined vibration feature to determine a blood pressure measurement for the subject 302.

In an embodiment, the real-time signal processing unit 318 is configured to perform vibrational cardiogram transformation to identify vibrational pulses corresponding with cardiac phase transitions. In a particular embodiment, the signal processing unit 318 implements the signal processing steps described in reference to FIG. 44 (described below).

The computing device 314 also includes a memory 322 for storing data generated by the real-time signal processing unit 318. The memory 322 stores the blood pressure measurement. The memory 322 also stores data used in the determination of the blood pressure measurement, such as raw vibration signal data, VCG waveform data, processed VCG waveform data, and vibration feature data.

The computing device 314 includes a user interface 324. The user interface 324 includes one or more software modules for presenting the blood pressure measurement generated by the real-time signal processing unit 318 in a human-readable format. The human-readable format may be a number or a visualization, such as a waveform or graph. The user interface 324 may be configured to continuously update to provide real-time blood pressure measurements as new raw vibration signals are acquired by the sensor module 304. The user interface 324 may also be configured to receive input data from a user, such as to make selections, change views or content presentation formats or styles, or send commands. The user interface 324 may include a control interface for controlling and viewing the performance and operation of the wearable sensor module 304.

The computing device 314 includes a display device 326. The display device 326 is configured to render and display the user interface 324. The display device 326 may include an input component, such as a touchscreen, for receiving user input.

The sensor interface computing device 314 is also communicatively connected to the data analytics server 328 via a data communication link 330. The communication link 330 may be any suitable wired or wireless communication link for transmitting and receiving data. The communication link 300 may be a satellite communication link. The communication link 330 may include a wide-area communication network, such as the Internet.

The data analytics server 328 includes a communication interface 336 for sending and receiving data to and from the sensor interface computing device 314. The data received by the communication interface 336 from the computing device 314 may include any data received or generated by the computing device 314. For example, the received data may include raw vibration signal data and/or blood pressure measurements.

The data analytics server 328 includes a post-processing unit 332 for performing post-processing on data received from the computing device 314. Post-processing may include determining a health state trajectory for the subject 302 based on the blood pressure measurement generated by the computing device 314. Post-processing may include analysis according to machine learning techniques. For example, machine learning techniques may include training a machine learning model and predicting on a trained machine learning model. Machine learning techniques may include unsupervised, semi-supervised, and supervised learning technique. In an embodiment, the post-processing unit may include a trained neural network including an input layer, at least one hidden layer, and an output layer configured to receive data from the computing device 314 as the input layer and generate a prediction at the output layer. In a specific example, the neural network may receive a blood pressure measurement at the input layer and generate health state trajectory data at the output layer. Input data may further include other vital sign measurements. In some cases, the post-processing unit 332 may be configured to apply fuzzy logic techniques to received data.

The data analytics server 328 includes a memory for storing the data received from the computing device 314 and the data generated by the post-processing unit 332, such as the health state trajectory.

The data analytics server 328 may be connected to one or more client computing devices (not shown) which may be used to access and view data generated by the server 328. For example, a client device may be used by an individual who is monitoring the health of the subject 302 (medical professional, command center for space or military operations).

Referring now to FIG. 5, shown therein is a computer system 400 for non-invasive blood pressure measurement, according to an embodiment. The computer system 400 includes a plurality of software modules configured to perform various operations and provide various functionalities described herein. The computer system 400 may be implemented as a single device or across a plurality of devices. In an embodiment, the computer system 400 may be implemented at the sensor interface computing device 314 of FIG. 4.

The computer system 400 includes a memory 402, a processor 404, a communication interface 406, and an output device (e.g. display device) 408. The components are communicatively connected via bus 409.

The communication interface 406 receives raw vibrational signal data 410 from a sensor device, such as wearable sensor module 304 of FIG. 4, configured to sense and detect vibrations corresponding to cardiac mechanical activity at the surface of the chest. The communication interface 406 may receive the data 410 and be configured to communicate with the sensor device using Bluetooth or other wireless connection.

The vibration signal data 410 is stored in memory 402. The raw vibration signal data 410 includes acceleration data 412 (derived from the acceleration component of the vibration signal) and rotational velocity data 414 (derived from the gyration component of the vibration signal).

The processor 404 includes a VCG waveform generator module 416 which receives the acceleration data 412 and rotational velocity data 414 from memory 402 and generates VCG waveform data 422 therefrom. The VCG waveform data 422 includes an SCG component (corresponding to the acceleration data 412) and a GCG component (corresponding to the rotational velocity data 414). The VCG waveform data 422 is stored in memory 402. The VCG waveform data 422 may include various distortions.

The processor 404 further includes a filtering and demodulation module 420 which receives the VCG waveform data 422 and demodulates the VCG waveform data 422 which may modulate the signal, such as sensor placement, respiratory activity, exertion, and motion artifact. The filtering and demodulation module 420 outputs a processed VCG waveform 426. The processed VCG waveform data 426 has an increased precision compared to the VCG waveform data 422. The processed VCG waveform data 426 is stored in memory 402.

The processor 404 further includes a vibrational pulse identifier module 424. Generally, the vibrational pulse identifier module 424 is configured to identify prominent vibrational pulses corresponding to cardiac phase transitions. The vibrational pulse identifier module 424 receives the processed VCG waveform data as input and determines the vibrational pulses V1 and V2, which correspond with the first and second primary heart sounds, respectively. The vibrational pulse identifier module 424 may then extract processed VCG waveform data 426 corresponding to the V1 and V2 (V1 and V2 VCG data 430) from the processed VCG waveform data 426. The V1 and V2 VCG data 430 is stored in memory 402.

The processor 404 further includes a vibration feature processing module 428. The vibration feature processing module 428 receives an output from the vibrational pulse identifier module 424 (e.g. V1 and V2 VCG data) and determines a vibration feature therefrom. The vibration feature is stored in memory 402 as vibration feature data 434.

The vibration feature processing module 428 may be configured to determine a jerk value from the linear acceleration component of the VCG data 430.

The vibration feature processing module 428 may be configured to determine an amplitude value from the linear acceleration component of the VCG data 430.

The vibration feature processing module 428 may be configured to determine a left ventricular ejection time (LVET) value from the linear acceleration component of the VCG data 430.

The vibration feature processing module 428 may be configured to determine a rotational kinetic energy (RKE) value from the rotational velocity component of the VCG data 430.

In some cases, the vibration feature processing module 428 may process both the acceleration component and rotational velocity component of the VCG data 430 to generate vibration feature data 434.

In some cases, the vibration feature is derived from valve motion (e.g. mitral valve, aortic valve opening and closing).

The processor 404 further includes a blood pressure waveform generator module 432. The blood pressure waveform generator module 432 receives vibration feature data 434 as input and determines blood pressure (BP) waveform data 438 therefrom. The BP waveform data 438 includes blood pressure values or measurements as a function of time. The BP waveform data 438 is stored in memory 402.

The processor 404 further includes a blood pressure (BP) measurement generator module 436. The BP measurement generator module 436 determines a discrete blood pressure measurement, such as a systolic pressure value, diastolic pressure value, or combination systolic and diastolic value (i.e. systolic pressure over diastolic pressure) from the BP waveform data 438. In an embodiment, the BP measurement generator module 436 may be configured to determine a systolic value and diastolic value for each cardiac cycle. The blood pressure measurements generated by the BP measurement generator module 436 are stored in memory 402 as BP measurement data 442.

The processor 404 further includes a user interface module 440. The user interface module 440 is configured to generate a human-readable format of the BP measurement data 442 or BP waveform data 438 for presentation to a user. The user interface module 440 is further configured to generate a graphical user interface including a plurality of user interface components for presenting data to the user, such as the blood pressure measurement in human-readable form, and receiving input data from a user.

In an embodiment, the user interface module 440 is configured to present BP waveform data 438 as a continuously updating waveform or graph. The waveform may be annotated with values, such as systolic and diastolic values. In another embodiment, the user interface module 440 is configured to present BP measurement data 442 as continuously updating or static systolic and diastolic values. The user interface 440 may update the BP measurement data 442 presented for each new cardiac cycle. In yet another embodiment, the user interface 440 may present both the BP waveform data 438 as a waveform and the BP measurement data 442 as systolic and diastolic values, one or both of which are updated at regular intervals (e.g. each cardiac cycle).

The user interface generated by the user interface module 440 is presented via the output device 408, which may be a display device or the like.

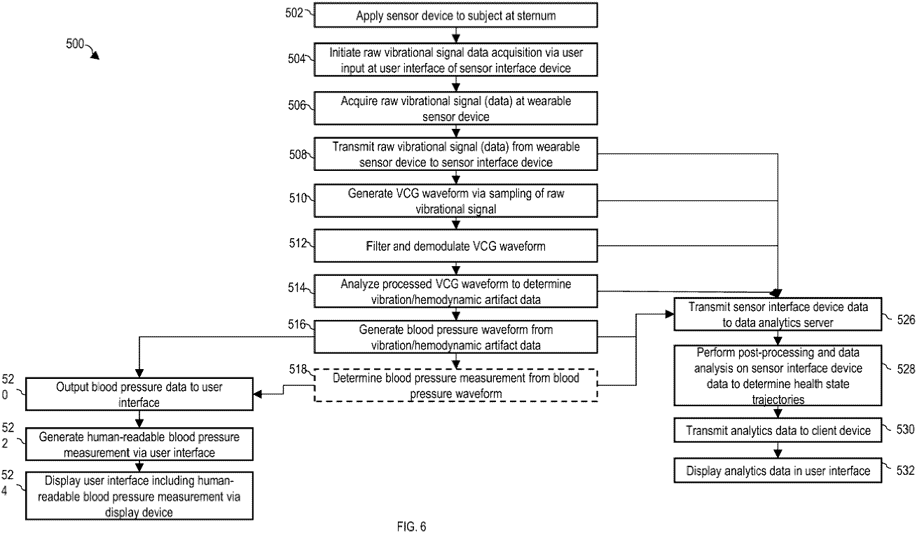

Referring now to FIG. 6, shown therein is a method 500 of non-invasive blood pressure measurement using the system 300 of FIG. 4, according to an embodiment.

At 502, the sensor device 304 is applied to the subject 302 at the sternum (xiphoid process).

At 504, the acquisition of raw vibration signals by the sensor device 304 is initiated by a user input. The user input may be provided by the subject 302 or by another individual. The user input initiating acquisition is provided via a user interface presented at the sensor interface computing device 314.

At 506, raw vibration signals are acquired by the sensor device 304. The raw vibration signals include a linear acceleration component and rotational velocity component.

At 508, collected raw vibration signal data is transmitted from the sensor device 304 to the computing device 314. In an embodiment, this data is transmitted using Bluetooth.

Steps 510 to 516 are performed by the computing device 314 and may be performed by the real-time signal processing unit 318.

At 510, a VCG waveform is generated by sampling the raw vibration signal.

At 512, the VCG waveform is filtered and demodulated to remove distortions.

At 514, the processed VCG waveform is analyzed to determine a vibration feature from the VCG waveform. In some cases, determining the vibration feature may be preceded by identifying or detecting the vibrational pulses corresponding to cardiac phase transitions (e.g. from systolic phase to diastolic phase, from diastolic phase to systolic phase). The vibrational pulses may correspond with the two primary heart sounds.

At 516, a blood pressure waveform is generated from the vibration feature data.

Optionally, at 518, a blood pressure measurement comprising a blood pressure value (e.g. systolic, diastolic, systolic/diastolic) is determined from the blood pressure waveform. This step may be performed in implementations where the user (i.e. the reader of the data presented via display 326) is a non-professional who would more easily process a non-waveform representation of the blood pressure measurement.

At 520, the blood pressure data, which may include one or both of the blood pressure waveform and blood pressure measurement, is provided to the user interface 324.

At 522, a human-readable representation of the blood pressure data is generated by the user interface 324 for presentation to a user.

At 524, the user interface 324, including the human-readable representation of the blood pressure measurement, is displayed at the display 326.

Steps 520 to 524 may be performed by the computing device 314. In variations, steps 520 to 524 may be performed by another computing device (e.g. a physician device) that receives the blood pressure data from the computing device 314.

At 526, data from the sensor interface computing device 314 is transmitted to the data analytics server 328. The transmitted data may include any one or more of raw vibration signal data, VCG waveform data, vibration feature data, blood pressure waveform data, and blood pressure measurement data.

At 528, the data analytics server 328 performs post-processing and data analysis on the received data to determine a health state trajectory for the subject 302. This may include processing and analysis of additional data not provided by the system 300 and/or related to other vital sign measurement data for the subject 302.

At 530, the analytics data generated by the data analytics server 328 is transmitted to a client device. The client device may be associated with a user who is monitoring the health status of the subject 302, such as a medical professional or space or military command.

At 532, the analytics data received at the client device is displayed in a user interface.

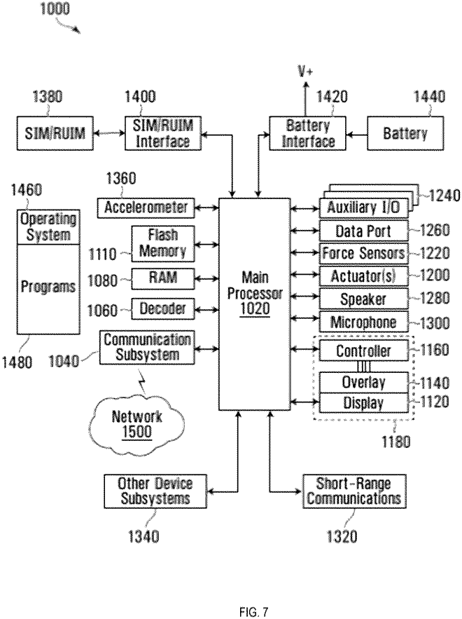

FIG. 7 shows a simplified block diagram of components of a device 1000, such as a mobile device or portable electronic device. The device 1000 may be, for example, any of devices 304, 314, 328 of FIG. 4. The device 1000 includes multiple components such as a processor 1020 that controls the operations of the device 1000. Communication functions, including data communications, voice communications, or both may be performed through a communication subsystem 1040. Data received by the device 1000 may be decompressed and decrypted by a decoder 1060. The communication subsystem 1040 may receive messages from and send messages to a wireless network 1500.

The wireless network 1500 may be any type of wireless network, including, but not limited to, data-centric wireless networks, voice-centric wireless networks, and dual-mode networks that support both voice and data communications.

The device 1000 may be a battery-powered device and as shown includes a battery interface 1420 for receiving one or more rechargeable batteries 1440.

The processor 1020 also interacts with additional subsystems such as a Random Access Memory (RAM) 1080, a flash memory 1100, a display 1120 (e.g. with a touch-sensitive overlay 1140 connected to an electronic controller 1160 that together comprise a touch-sensitive display 1180), an actuator assembly 1200, one or more optional force sensors 1220, an auxiliary input/output (I/O) subsystem 1240, a data port 1260, a speaker 1280, a microphone 1300, short-range communications systems 1320 and other device subsystems 1340.

In some embodiments, user-interaction with the graphical user interface may be performed through the touch-sensitive overlay 1140. The processor 1020 may interact with the touch-sensitive overlay 1140 via the electronic controller 1160. Information, such as text, characters, symbols, images, icons, and other items that may be displayed or rendered on a portable electronic device generated by the processor 102 may be displayed on the touch-sensitive display 118.

The processor 1020 may also interact with an accelerometer 1360 as shown in FIG. 7. The accelerometer 1360 may be utilized for detecting direction of gravitational forces or gravity-induced reaction forces.

To identify a subscriber for network access according to the present embodiment, the device 1000 may use a Subscriber Identity Module or a Removable User Identity Module (SIM/RUIM) card 1380 inserted into a SIM/RUIM interface 1400 for communication with a network (such as the wireless network 1500). Alternatively, user identification information may be programmed into the flash memory 1100 or performed using other techniques.

The device 1000 also includes an operating system 1460 and software components 1480 that are executed by the processor 1020 and which may be stored in a persistent data storage device such as the flash memory 1100. Additional applications may be loaded onto the device 1000 through the wireless network 1500, the auxiliary I/O subsystem 1240, the data port 1260, the short-range communications subsystem 1320, or any other suitable device subsystem 1340.

For example, in use, a received signal such as a text message, an e-mail message, web page download, or other data may be processed by the communication subsystem 1040 and input to the processor 1020. The processor 1020 then processes the received signal for output to the display 1120 or alternatively to the auxiliary I/O subsystem 1240. A subscriber may also compose data items, such as e-mail messages, for example, which may be transmitted over the wireless network 1500 through the communication subsystem 1040.

For voice communications, the overall operation of the portable electronic device 1000 may be similar. The speaker 1280 may output audible information converted from electrical signals, and the microphone 1300 may convert audible information into electrical signals for processing.

Further principles for determining blood pressure using VCG implemented by the systems and methods described herein will now be described.

The present disclosure seeks to provide systems and methods that describe how sternal vibrations are related to the motion of the valves in the heart, and how this valve motion is caused by the cardiac blood pressure cycle. The systems and methods described herein, such as systems 300 and 400 of FIGS. 4 and 5 respectively, are configured to calculate central aortic blood pressure during each cardiac cycle by deriving it from VCG signal morphology. The systems and methods described herein may provide a non-invasive, central, aortic pressure measurement.

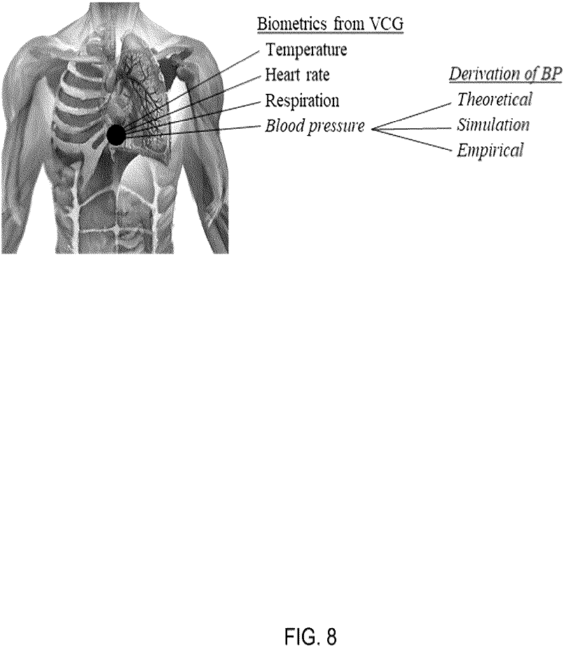

FIG. 8 illustrates biometric measurements that may be calculated from the VCG signal. Blood pressure determination may include any one or more of theoretical (signal processing), simulation (3D modelling), and empirical (neural network) analysis.

The VCG signal detected by the systems and methods of the present disclosure can be used to calculate one or more biometric measurements including blood pressure. The determination of blood pressure from the VCG signal may include any one or more of signal processing, simulation or 3D modelling, and empirical analysis. Empirical analysis may include analysis via one or more machine learning techniques, such as a neural network. In some cases, the systems and methods described herein may determine additional biometrics from the VCG signal, such as temperature, heart rate, and respiration.

We have already identified the process of analyzing VCG waveforms from the motion detected by an inertial measurement unit, including an algorithm therefor. The algorithm was used to measure heart rate from an SCG signal. It was further developed to measure heart rate from a VCG signal by combining SCG and GCG. These results were based on an accurate detection of the vibrational pulse corresponding to the first heart sound in each cardiac cycle. The timestamp of this pulse in each cardiac cycle provided a marker from which to calculate heart rate. Recently, the algorithm was extended to detect the vibrational pulse corresponding to the second heart sound. The algorithm, which may detect vibrational pulses corresponding to cardiac phase transitions, may be used by the systems and methods of the present disclosure (e.g. implemented via one or more computer components) to analyze vibrational pulses and determining a cardiac hemodynamic measurement.

Furthermore, we have also investigated the modulation of VCG by respiratory activity and empirically classified lung volume states. The effect of body orientation on HR measurement was also investigated.

The vibrational pulses corresponding to the two heart sounds are generated by the mechanical motion of cardiac valves. The valves are hydraulically controlled by blood pressure differentials in the heart. Therefore, the detection of both vibrational pulses will provide the basis for calculating blood flow through the heart. An analysis of the pulses will lead to the calculation of blood flow.

The entire system may be simulated in Comsol to verify theoretical results. As a first step, we are currently simulating the propagation of vibrational waves from the valves to the sternum. We have already produced preliminary results for this simulation of vibrational wave propagation through the chest. The work is currently being developed into full study.

In an empirical study, we are using neural networks to establish a direct correlation between VCG waveforms and real-time blood pressure measurements obtained from a finger cuff. We have already established a preliminary correlation between the two and are working to improve the accuracy of these results by cleaning the input data and modifying the network architecture. Its associated journal article is being written as an equal authored paper. The expected submission date is 1st July 2020.

Finally, the modulation of vibrational wave propagation by respiration and sedentary movement will be further analyzed.

### Jerk Artifact

Referring again to FIG. 1, a schematic representation of the structure of the human heart 100 is shown. The section of interest, for the moment, is the left atrium (120) and left ventricle (140) with their associated valves mitral (123) and aortic (141).

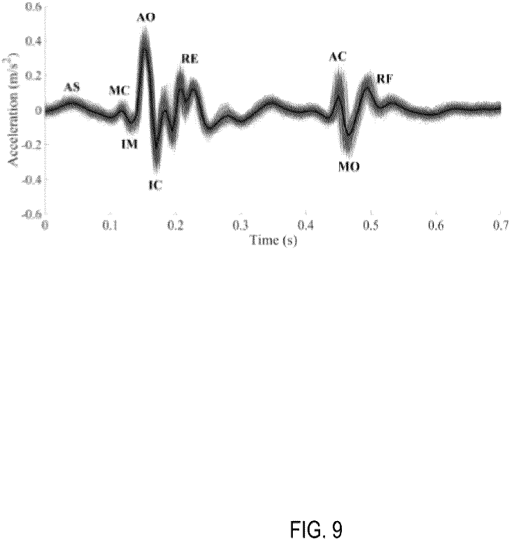

Analytical Approach. The research done to date indicates that the vibrations detected by the VCG corresponding with the ‘first heart sound’ are caused by the closure of the mitral/ tricuspid valves (atrioventricular valves - AV). This first heart sound is generated when sudden closure of AV valves (MC component of the SCG trace shown in FIG. 9) results in oscillation of the blood in the ventricles, which causes vibrations. Once the left ventricle has compressed and ejected the blood into the aorta, the aortic valve closes as a result of a reversal of the energy gradient of blood across it induced by the relaxation of the left ventricle and a commensurate fall in intraventricular pressure. This abrupt valve closure (AC on the SCG tract) causes the second heart sound’. The first heart sound indicates the end of the diastolic and start of the systolic phase of the cardiac cycle and the second heart sound indicates the end of the systolic and start of the diastolic phase.

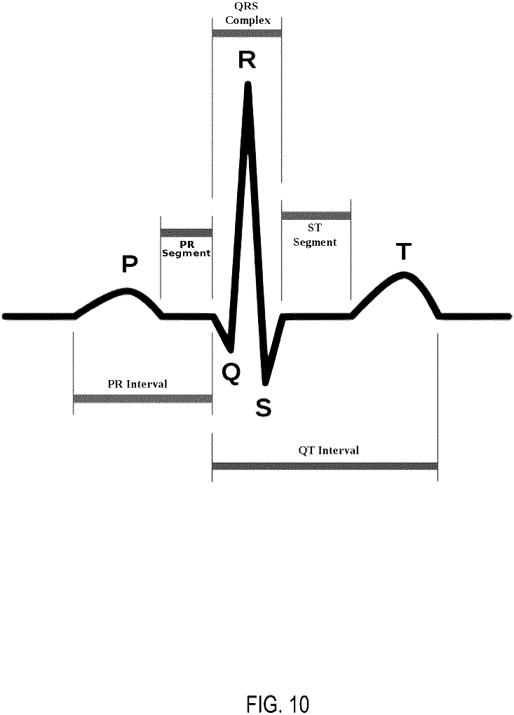

The pumping action of the left ventricle 140 closely resembles a diaphragm pump and the action of the mitral and aortic valves 123, 141 is governed by pressure differentials rather than electrical commands. The contraction of the ventricles (both right and left) is driven by the ‘QRS’ complex of the ECG (see FIG. 10) which is an electrical command but the valve operation is a consequence of the pressure generated by the contraction.

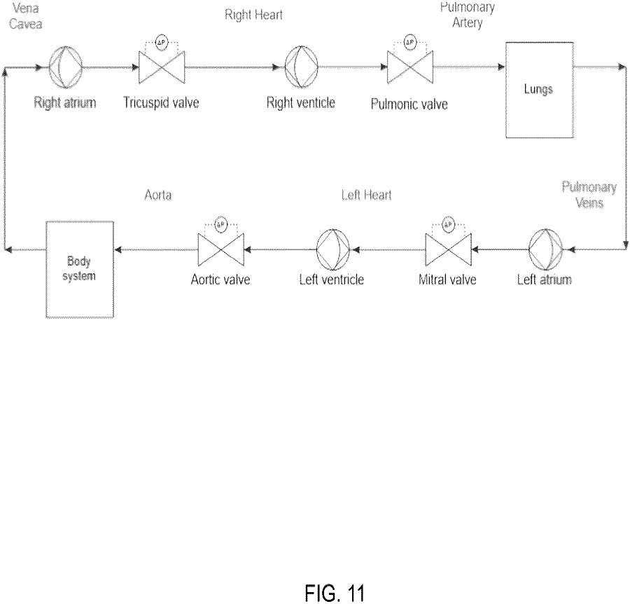

FIG. 11 is a schematic representation of the blood circulation circuit. The schematic representation accurately indicates that there is no ‘storage’ or ‘reservoir’ for the blood within the cardio-vascular system. This means that as oxygen demand increases (due to, for example, physical exertion), the entire system must increase its cadence which results in a combination of increased heart rate and respiration volume with consequential dynamic responses in blood pressure. A decrease in demand (i.e. body at rest) results in a decrease in heart rate and further dynamic responses in blood pressure.

Given that direct measurement of the blood pressure across the aortic valve, that is, in either the left ventricle or the aorta, is not practical (very invasive; not acceptable for long term monitoring outside the hospital context), a proxy measurement that can be correlated to the blood pressure is needed. In this regard, embodiments of the systems and methods of the present disclosure focus on the vibration (measured as linear acceleration and rotational velocity) associated with the opening and closing of the valves on the left side of the heart and the consequential movement of blood. The quantification by the system of the vibration produced by the blood flow caused by the ventricle’s contraction will lead to establishing blood pressure as a proportional value relative to the force (acceleration) ejecting the blood into the aorta. Several attributes (called artifacts or features) of the acceleration signal, including any one or more of amplitude, rate of change in acceleration (third order derivative of position called ‘jerk’) and event durations, such as the duration of blood ejection from the ventricle into the aorta called Left Ventricular Ejection Time (LVET), may be derived and/or used by the systems and methods of the present disclosure to determine a continuous blood pressure measurement (or other hemodynamic measurement).

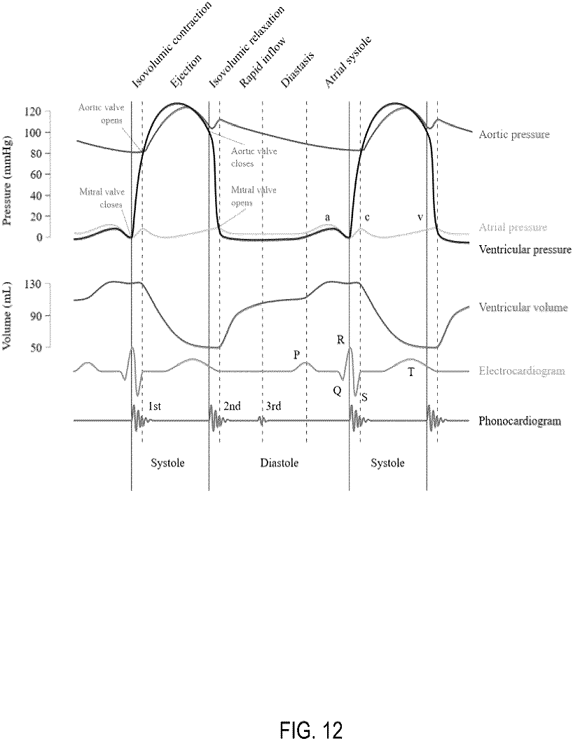

FIG. 12 is the cardiac cycle diagram (sometimes called a Wiggers diagram) which presents the blood pressure in relation to the physical movements of the heart and its electrical commands.

The top trace in FIG. 12 represents the Aortic pressure which is related to the pressure read by sphygmomanometers (i.e. blood pressure machines). The point at which the rising ventricular pressure crosses the aortic pressure which causes the aortic valve to open, is the value reported as the diastolic pressure. The top of the ventricular pressure trace (maximum ventricular pressure) which occurs at about the mid-point in the left ventricular ejection period is the value reported as the systolic pressure.

The rhythmic rise and fall of the blood pressure in the aorta is related to the periodic injection of a mass of blood by the left ventricle (the stroke volume). The majority of the kinetic energy of this mass of blood is passed though the vascular system and promotes immediate blood flow while a portion of the kinetic energy is absorbed by the elasticity of the aortic wall to be later returned to the blood and support circulation. Some fraction of the kinetic energy is transferred to the thoracic structures supporting the heart and aorta. It is this last fraction, in the form of vibrations, mostly low sub-acoustic, that is detected by the inertial motion sensor (accelerometer and gyroscope) we locate on the xyphoid process and is reported as the vibrational cardiogram (seismocardiogram and gyrocardiogram, respectively).

The problem of measuring the systolic and diastolic pressures by monitoring the vibrations on the sternum is twofold. Firstly, we must discover the exact mechanism by which the vibrations are generated as well as discover/ quantify the fraction of kinetic energy that is converted to vibration. Secondly, we must understand the transmission path through which the vibrations travel to reach the sensor in order to evaluate how the travel has modified the signal. We believe that the application of Fluid-Structure Interaction (FSI) analysis which combines elements of Computational Fluid Dynamics (CFD) and Finite Element Analysis (FEA) can provide answers that will ultimately allow us to establish the analytical relationship between vibrations measured on the thorax and cardio-pulmonary properties such as heart rate, respiratory rate and blood pressure.

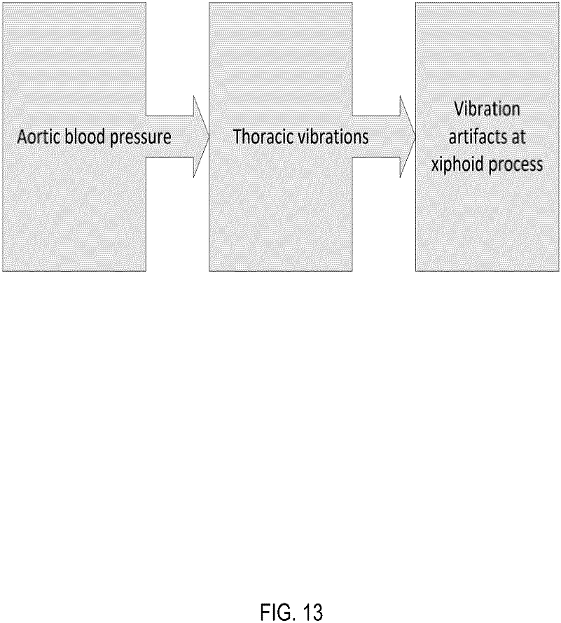

Machine Learning Approach. The analytical approach to establishing a link between aortic blood pressure and vibration measurements at the xiphoid process, as described in the previous section, can be represented as the block diagram in FIG. 13. It consists of two transformations. The first is the conversion of aortic blood pressure to thoracic vibration. The second is the relationship between the vibration artifacts (or vibration features) measured at the xyphoid process and the vibration signal at its source (at or near the heart).

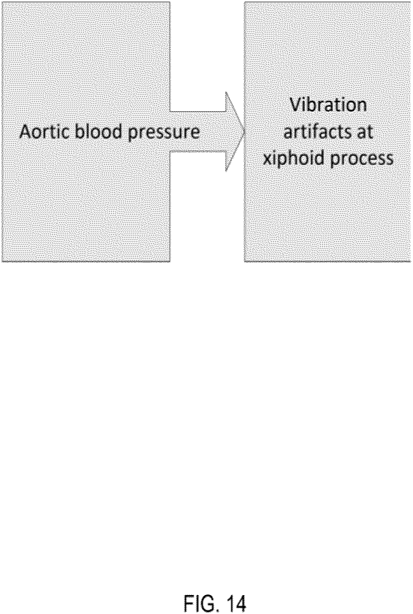

In contrast, the application of machine learning to the problem of determining blood pressure from vibration artifacts at the xyphoid process follows the block diagram in FIG. 14.

The machine learning approach is, in essence, a black box that looks at the characterising data it is given and determines the best correlation it can between the desired outcome (aortic blood pressure) and the input data provided (vibration artifacts).

Given our desire to understand more completely the relationship between vibration and blood pressure (as evidenced by the effort expended to date on the analytical approach), our application of machine learning to the blood pressure problem focuses on using the technology to analyse our data and surface correlations and anomalies that are not currently known to us. Machine Learning, in this context, is more a tool to support discovery and data analysis than a means to a solution. We intend to investigate the correlations and anomalies in order to hopefully, gain better insights into the relationship between vibration and blood pressure and advance our analytical efforts.

Additional features of the systems and methods for non-invasive blood pressure measurement will now be described. Such features may be implemented by the systems and methods of the present disclosure, such as those described in FIGS. 2-6. In some cases, systems and methods for non-invasive physiological activity monitoring are described. It is to be understood that such systems and methods may include the systems and methods for blood pressure measurement described in the present disclosure, or portions thereof, which may be in addition to other physiological activity or vital sign monitoring features.

Non-invasive Physiological Activity Monitoring System (NIPAMS).

### 1. Introduction

### 1.1 Project Overview

This project addresses the urgent need for accessible health monitoring on earth and in space. In the context of space-flight missions, traveling long distances makes for difficulty in real-time communication with earth. In the event of a medical emergency, communication with the flight surgeon may be strained or impossible. This gap in communication has spurred public space agencies worldwide, including the Canadian Space Agency (CSA), to identify the need for an on-astronaut wireless biometric monitoring technology in order to support the crew members. Their requirement has motivated our collaboration, which proposes the development of an autonomous system that can identify symptoms, diagnose or predict health states, match treatment options, and transmit the information to a base (e.g., space station, clinic, hospital). The plan is to build a wearable, wireless sensing platform that monitors, records, and analyzes key physiological parameters. Our work is motivated by the prospect of ushering in an age of accessible health, wellness, and fitness technology solutions by leveraging the space community’s imperative to provide computerized healthcare to astronauts. Our achievement of these objectives will produce long-term exploratory results and short-term technological solutions, both of which are valuable to MDA Corporation and the Canadian biotechnology, health, wellness, fitness, defense, and space sectors.

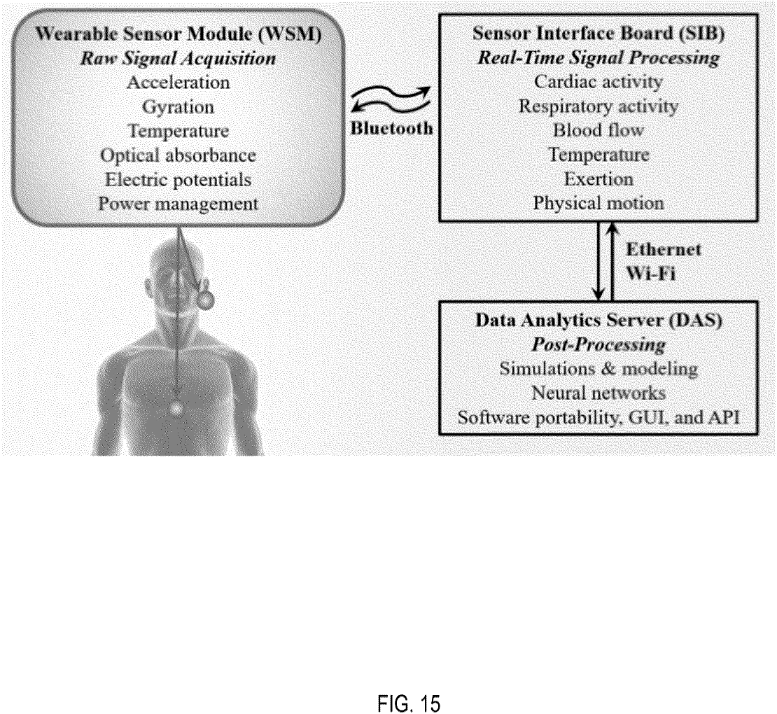

The reported work describes the development and demonstration of a Non-invasive Physiological Activity Monitoring System (NiPAMS) that uses digital signal processing (DSP) algorithms to convert electro-optical and electro-mechanical sensor signals into physically verifiable, real-time measurements of physiological states and their response to associated physical activity. A system schematic is shown in FIG. 15. Attached to the user, purpose-built Wearable Sensor Modules (WSMs) acquire either inertial measurements (IM) using an accelerometer and a gyroscope, or optical measurements using light emitting diodes and a photodetector. A portable Sensor Interface Board (SIB) wirelessly pairs with multiple WSMs to perform real-time DSP on the sensor signals and calculate metrics associated with physiological activity (e.g., cardio-respiratory activity, blood flow) and physical activity (e.g., movement). The data will be monitored through a native software interface. The SIBs transfer raw signals and measurements to a central Data Analytics Server (DAS) for further analysis, recording, and predictions. The NiPAMS is being developed via a strategic partnership between the Plant group at McGill University, and MDA, an Ontario-based space systems and sensors engineering company.

FIG. 15: Schematic of the NiPAMS. The system will support an IM-WSM for acceleration and gyration, and an OM-WSM for light absorption. Both WSMs will communicate via Bluetooth. WSM signals sent to the SIB will be converted to biometric readings, and then relayed to the DAS. The DAS will store and analyze measurements for the evaluation of health state trajectories.

### 1.2 Physiological Measurements

The NiPAMS will primarily monitor the four vital signs – heart rate, respiration rate, blood pressure, and body temperature – to evaluate a subject’s health in real-time. Additionally, it will calculate key physiological parameters related to cardio-respiratory activity, movement, exertion, oxygen saturation, and hemodynamic homeostasis, and then decouple these measurements from each other to deliver comprehensive evaluations of health, wellness, and fitness. The accuracy of these results will be benchmarked against clinical standards. The NiPAMS will therefore deliver the following measurements that are relevant to the evaluation of physiological activity:
Cardio-respiratory activity (CRA):
   Respiratory activity: rate (RR), volume (RV), phase, peripheral oxygen saturation (SpO2), lung capacity
   Cardiac activity: rate (HR), efficiency, HR variability, left ventricular ejection time and fraction, stroke volume, beat-to-beat duration (T_{BTB})
   Blood flow: systolic pressure (P_{Sys}), diastolic pressure (P_{Dia}), ejection velocity, viscosity
Body: surface body temperature (BT), physical exertion level (EL)
Physical motion: Motion capture (MoCap) information for motion artifact (MoArt) cancellation

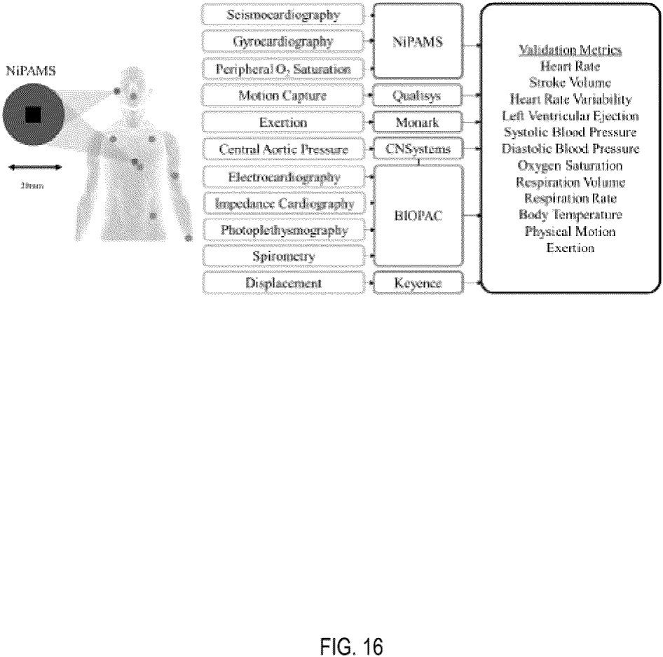

The system development has already demonstrated HR and RR measurements resulting in two journal articles, one conference paper, and one patent application. Considering these achieved milestones, the current objective is to estimate blood pressure. Toward this goal, we have built a NiPAMS laboratory located in room 814A of the McConnell Engineering Building at McGill University. The laboratory accommodates a physiological monitoring testbench including a computer, massage bed, and gold-standard measurement equipment as outlined in FIG. 16 for the purpose of comprehensive testing. These tests were designed to monitor any biometrics relevant to BP so that relationships between the NiPAMS signals and physiological measurements may be established.

FIG. 16: Equipment used for physiological measurements in the NiPAMS laboratory.

### 1.3 Roadmap to Blood Pressure

Cardio-respiratory activity generates vibrational waves that propagate through the chest and manifest as vibrations on the surface of the skin. These waves were recorded near the xiphoid process of the sternum due to its proximity to the heart and lungs. The vibrations were detected by an inertial measurement unit (IMU) attached on the skin. The content of the acquired signal consisted of a combined pneumatograph and vibrational cardiography (VCG) measurement. Our work attempts to describe how these sternal vibrations are related to the motion of the valves in the heart and how this valve motion is caused by the cardiac blood pressure cycle. The objective of our study is to provide calculations of central aortic blood pressure (BP) during each cardiac cycle by deriving it from VCG signal morphology. Any distortions caused by sensor placement, respiration volume, cardiac contractility and heart rhythm will be demodulated to improve the precision of the VCG waveform. This report describes the work completed and in progress toward the realization of central, aortic blood pressure at the sternum.

An accurate calculation of blood pressure using exclusively VCG waveform analysis requires a comprehensive understanding of the testing environment. This includes the physical characteristics of each subject, their cardiorespiratory activity including its physiological influences, variations of the thorax that modulate vibrational waves propagating to the sternum, the placement of the sensor, transient response and sensitivity of the sensor, system acquisition logistics, and the testing conditions for each subject. The development of the NiPAMS sensing system toward real-time, wireless functionality is described in Section 2. Each upgrade of the system was directly evaluated in subject trials. These trials were designed to measure the effect of lung volume, sensor placement, exertion, and MoArt as explained in Section 3. Each test acquired a collection of signals by using the equipment outlined in FIG. 16. The raw data comprising these signals were filtered to extract relevant biometric measurements as elaborated in Section 4, by using commercially available Biopac software and custom-built Matlab algorithms. Once filtered and demodulated, the processed VCG waveform could then be analyzed to extract measurements related to the cardiac pressure cycle. However, this requires knowledge of the relationships contained in the experimentally obtained VCG waveform morphology. Section 5 presents the current progress toward deriving a physically valid explanation of pressure-induced VCG morphology based on analytical equations and cross-verified with signal processing results. This model is being developed in parallel with simulations of pressure-induced vibrational wave propagation through the chest that are explained in Section 6. Furthermore, the relationship between central aortic pressure and VCG morphology is also being investigated via artificial intelligence algorithms in Section 7 in order to inform, or enhance, the predictions of the models. The results, limitations, and future plans are summarized in Section 8 along with a proposed timeline for 2020 that includes potential publications.

### 2. System Development

As mentioned earlier, this project in concerned with accessible healthcare on earth and space. A major motivation for upgrading the previous system was achieving remote monitoring. By upgrading to a remote senor, we could easily conduct more complicated tests such as the dynamic exercises performed during MoCap (Section 3.4) and not worry about cable lengths. Both previous and current systems were assembled from commercial, off-the-shelf components. In addition to being wireless, our system needed to be small, robust and achieve a decent sampling rate that matched or improved on the previous system.

### 2.1 Previous System

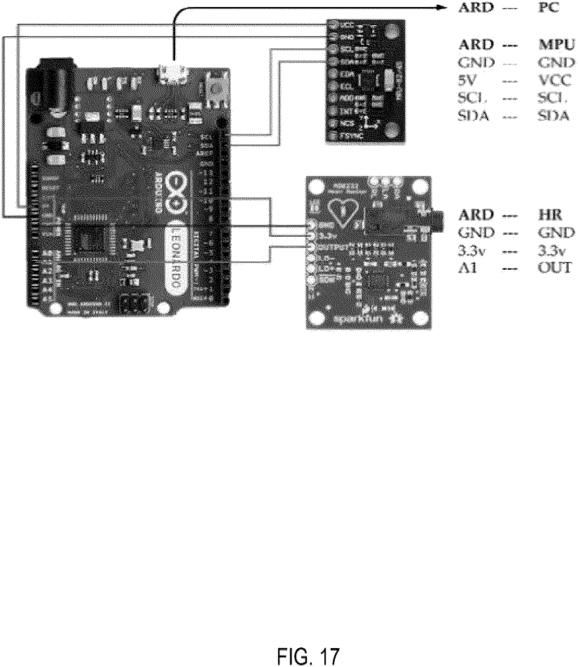

Cardiac-induced vibrations were detected by an inertial measurement unit (IMU) placed at the xiphoid process of the sternum. The IMU sensor is a nine-axis InvenSense Motion Processing Unit™ (MPU) 9250 (San Jose, CA, USA) consisting of a MEMS gyroscope and accelerometer, along with a digital compass that was not used in this work. The sensitivity of the MPU-9250 accelerometer and gyroscope were set at ±2 g and ±250 °/s respectively. A simultaneous ECG measurement was acquired by a SparkFun AD8232 Single Lead Heart Rate Monitor (Niwot, CO, USA). Both sensors were connected to an Arduino Leonardo microcontroller, as shown in the system configuration diagram of FIG. 17. The microcontroller was strapped around the subject’s torso near to the sensor. Sensor signals were transmitted to a computer via a USB cable, which also powered the system. The sampling rate of the Arduino was approximately 250 Hz. Data acquisition and signal processing were conducted by a custom built, MATLAB-based graphic user interface (GUI).

### FIG. 17: System Configuration Between the MPU-9250, AD8232, and Arduino Leonardo

As an external reference for the system, three heart rate measurements were recorded using an Omron 10 Series oscillometric blood pressure monitor at the brachial artery over a period of approximately three minutes during testing. The measurements obtained by the cuff were entered into the GUI manually during data acquisition.

### 2.2 Current System

### 2.2.1 Configuration

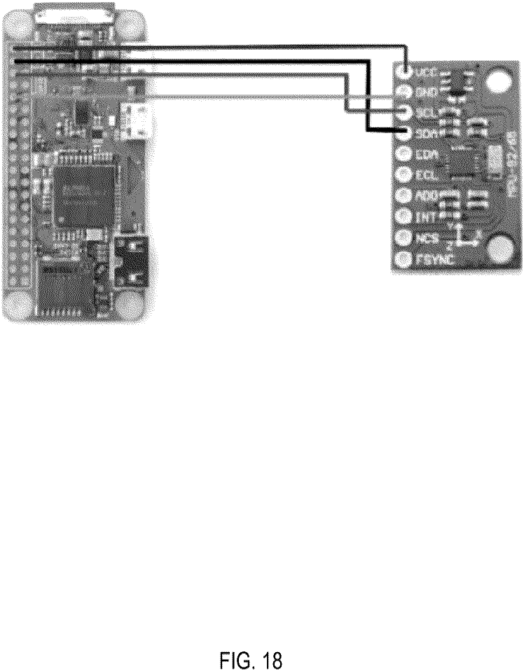

The main modification featured in the current system was a different micro-controller. The Raspberry PI (RPI) Zero W was used to control the system. This RPI model is a wireless micro-controller. The ICM-20602 six-axis InvenSense Motion Processing Unit™ replaced the MPU-9250 because of its discontinuation. The register architectures are similar between both models, which allowed for the ICM-20602 to be integrated into the system without any modifications to the code. However, the current system also used the MPU-9250 for the sake of continuity between system versions and the ICM-20602 was kept as backup. The RPI employed a PIZ Uptime battery shield to power the pi and provide wireless mobility to the user. The RPI connected to both IMU models on the l2C bus using hexadecimal addresses hard-coded into the hardware. The BIOPAC clock, which aided post-acquisition synchronization between IMU acceleration/gyration values and BIOPAC data, was inputted to the RPI using Programmable General-Purpose Input Output (GPIO) pins. The battery used was a LI-Ion Rechargeable Battery that could be recharged by connecting the shield to a PC using a USB-micro USB cable. As shown in FIG. 18, GPIO pins 1 (purple), 3 (black), 5 (red), 9 (green) were used for the I2C connection to the sensors. Pin 11 had been programmed for the connection to the BIOPAC. The sampling rate for the new system was approximately 560 Hz. Data acquisition and signal processing were controlled by a custom built, web-based user interface. The RPI acquired values from the IMU as well as the BIOPAC connection and appended the data to a text file on the Micro SD card.

### FIG. 18: System Configuration Between the MPU-9250, RPI, and Battery

TABLE 1

Hardware Requirements

| | |
|---|---|
| Hardware | Purpose |
| Raspberry PI Zero W | Micro-controller |
| PIZ Uptime | Battery Shield |
| LI-Ion Rechargeable Battery | Power Source |
| Micro SD Card | Raspbian OS & Storage |
| MPU9250, ICM20602 | InvenSense IMUs |

TABLE 2

Software Requirements

| | |
|---|---|
| Software | Purpose |
| Putty & Bonjour Services | Software required to SSH on Windows OS |
| FileZilla | Software required to transfer files b/w PC and RPI |
| Pip, I2C-tools, Flask-socketio, RPi.GPIO | Package installations required on Raspbian OS |
| Smbus, IO, Time, Logging, Threading, Matplotlib | Python libraries required by the application |

TABLE 3

System Specifications

| | |
|---|---|
| Specification | Value |
| RPI Processor speed | Single-core 1 GHz CPU |
| Maximum achieved sampling rate | ~560 Hz for one sensor |
| Battery life | 800 mAh, outputs 150 mA for 5+ hours |
| Dimensions | 65 mm long, 30 mm wide |

### 2.2.2 Dual Sensor Set-up

In order to study motion artifact in VCG signals, a dual sensor set-up was developed prior to the MoCap tests which will be discussed in section 4.4. The first sensor was attached at the xiphoid process of the sternum and the second sensor was attached on the back. The second sensor was at an equal vertical height from the ground as the first IMU in order to form a perpendicular distance between the sensors. Both sensor signals would carry motion information with the first sensor signal also carrying cardiac-induced vibrations. By using both signals in a manner similar to a differential amplifier, we can filter out the motion. To be able to read from both sensors which have the same hard-coded address (0×68), an extra connection from the IMU to the RPI was added. By connecting the AD0 pin on the MPU-9250 to either ground or 3.3 V, we can change the address of the sensor on the l2C bus. By grounding one sensor and setting the other to 3.3 V we can now interact with both sensors parallelly using addresses 0×68 & 0×69 respectively. It is important to note that the addition of a second sensor reduces the sampling rate to ~270 Hz, which is almost half of the sampling rate achievable with one sensor.

### 2.2.3 Acquisition Interface

There were two versions of the interface used in data acquisition. The first version was a web-based application that could plot the IMU data at the end of polling the sensor. The execution was sequential and was based on the following steps: (i) the webserver loads upon typing the RPI’s IP address in a browser, (ii) the IMU is polled when a runtime is inputted in the URL, (iii) the RPI appends the raw data to a text file and (iv) finally generates a plot from the acquired information. Upon generating the plot, it was saved as an image and sent to a website local to the network. Advantages of this version included a simple execution that does not involve parallelization (multi-threading), and a very high sampling rate of ~560 Hz. There were two

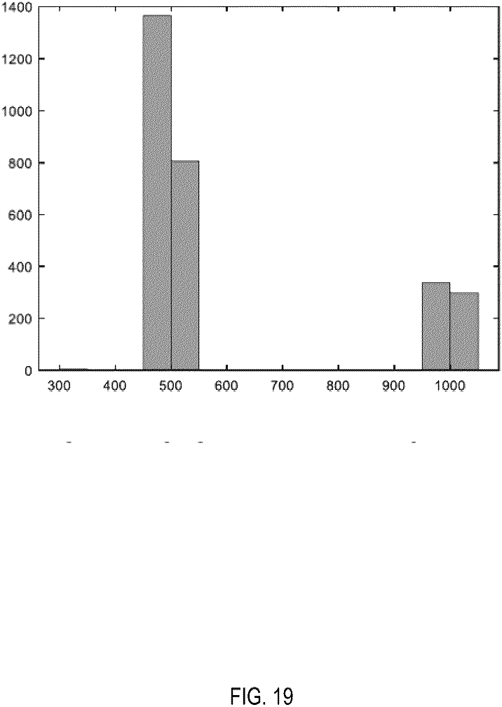

drawbacks regarding this version; a delay between tests due to waiting for the RPI to finish updating the text file, and inability to apply real-time plotting to the system. As shown in FIG. 19, a histogram of the sampling rate was generated. The plot shows the difference between every two timestamps collected from the IMU over a total of 5 seconds. The bulk of the values lie around ~500 Hz with very few instances of ~300 Hz.

### FIG. 19: Histogram of sampling Rate Over 5 Seconds – Sequential Method

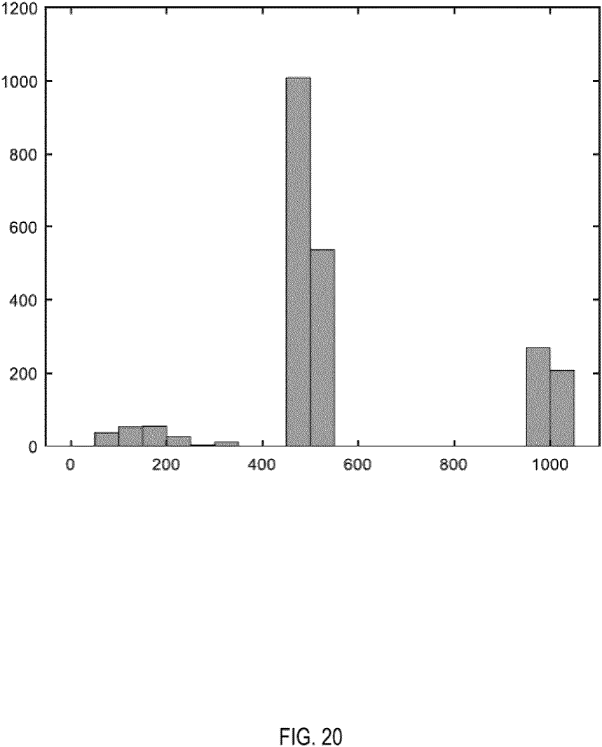

The second version was also a web-based application which loaded upon typing the RPI’s IP address into a browser. The execution was multi-threaded with two threads; polling, which polled the IMU for data, and printing, which appended the data to the text file. A counter and a delay ensured the printing thread never exceeded the polling thread. This version of the interface did not include real-time plotting. Instead, the printing thread sent the IMU raw data in real-time to be displayed on a local website. The data can be sent per-timestamp or in batches and performance improved linearly with batch size. The advantages of this version included data storage and transmission in real-time as well as a future option for real-time plotting. A drawback of this version was the periodic drops in sampling rate that occurred due to higher computational demand on the RPI. This resulted in small time periods where the sampling rate was very low (~50 Hz) hence lowering average sampling rate. As shown in FIG. 20, a histogram of the sampling rate was generated. The bulk of the values lie around ~500 Hz with a significant number of instances in the 50-200 Hz range.

### FIG. 20: Histogram of Sampling Rate Over 5 Seconds – Multi-Threading Method

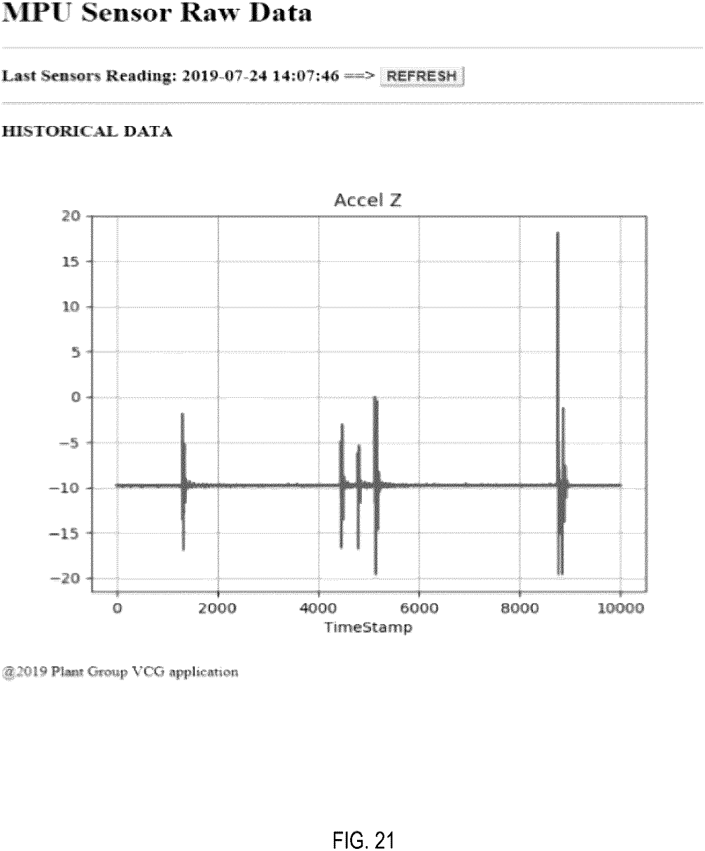

An instance of the plots produced by the web-based interface is displayed in FIG. 21. The web-interface was designed for quick plotting and visualization. It is important to note that while these versions of the interfaces were rigorously tested, no tests have been run for longer than 20 minutes. In order to check the integrity of the data and to determine the magnitude of the drift in clock as runtime increases, the RPI was set to only poll and append to the text file in real-time, without starting a webserver, for 12 hours. Unfortunately, the test only lasted for 1.7 hours before the RPI killed the task. The clock employed was a simple square wave with a periodically varying width. The integrity of the clock signal was maintained for 1.3 hours but was distorted afterwards. This problem is yet to be fully investigated.

### FIG. 21: Simple VCG Front-End Application

### 2.2.4 Real-Time Plotting

The two versions of the application discussed above satisfied the needs of the project with regards to data integrity for analysis, and protocol simplicity for testing. However, there is further room for improvement. Currently, there is a version of the system where generating the plot and uploading the image could be done in real-time. However, this was performed by the RPI which drastically lowered the sampling rate. Hence, the next task would be exporting the graphing and visualization of data to the client (PC) and instead, have the RPI (server) only pass the input received from the IMU. This would maintain a high sampling rate and allow greater flexibility in the user interface.

An implementation of a client-server socket program was developed in Python. The RPI was the server and the receiving PCs were the clients. Transmission Control Protocol (TCP) was employed for communication between sockets. TCP was initially chosen to ensure lossless data receipt on the client side. A loss of samples would negatively affect the ability to visualize the data and could lead to difficulties in analysis. In the future, User Datagram Protocol (UDP) will be investigated to determine if it provides significant sampling rate improvement and if this enhanced performance will be worth the trade-off with potential data loss.

As mentioned earlier, sockets were used to transfer the polling data from the RPI to the PC. The only way the client could connect to the RPI was by connecting to the server’s IP address under the same network. By default, the RPI’s IP address changed every time it connected to a PC wirelessly. Therefore, the IP address needed to be manually updated on the client-script once a connection was established. One solution involved setting a static IP address for the RPI. In a commercial or Space application, the static IP approach would be feasible given the topology of the solution would be known a priori. However, during development, where there are several devices connected to the network, there was a possibility another device would have an identical IP address. This would result in errors as the PC would never know which device is in question. To address this, a script that pinged raspberrypi.local to acquire the IP address was developed on the PC-side. Since our RPI model supported multicast-DNS, it could be reached by using its hostname and the .local suffix. The default hostname on a fresh Raspbian install is raspberrypi, so by default any RPI running Raspbian responds to raspberrypi.local. The script would then grab the specific IP string and use that string to initiate a connection. In the case of an RPI with the same identifier connected to the network, a name change would have to be made.

### 2.3 Conclusion

As discussed earlier, the present versions of the application adequately served the objectives of the team. The system achieved a high sampling rate of ~560 Hz for one sensor and ~270 Hz for a dual sensor configuration. The testing protocol is a simple two-click process where runtime is entered into the application, and a plot is displayed for debugging purposes. There is also potential for real-time plotting functionality. Future work includes developing a start-stop option for plotting, a fully developed GUI for user friendliness (zooming and scrolling), and a UDP vs. TCP performance investigation.

### 3. Experimental Work

Vibrational cardiography represents a complex problem merging within the fields of physiology and signal processing. In order to build robust DSP algorithms, the experimental procedures for must account for the inconsistencies of the human body. Each set of experiments was designed to either isolate specific parameters, or statistically generalize unknown variables. The pilot study of the experimental work is described in section 3.1 where data was recorded with an IMU, ECG, and an Omron blood pressure monitor. These results were used to design the trial described in section 3.2 where a large number of subjects were recorded extensively with the IMU and BIOPAC systems. The third study in section 3.3 produces a pilot study to investigate the effect of positioning on VCG waveforms. The final study in section 3.4 explores the effect of orientation and movement artifacts in the VCG signal.

### 3.1 VCG Omron

Testing was conducted with approved protocols in accordance with the Review Ethics Board at McGill University. The biometric signals of 25 male subjects between the ages of 20 and 30 years old were measured. These subjects had no known cardio-respiratory ailments. The testing protocol consisted of two tests that lasted approximately seven minutes in duration. The first test involved each subject resting supine. One minute after the start of ECG and VCG data acquisition, an Omron sphygmomanometer cuff monitor was activated. Three consecutive measurements were performed using the cuff during the seven-minute duration of the testing cycle. The cuff also measured the baseline heart rate of the participants while they were at rest. Following this test, the subjects performed a high intensity floor exercise known as the mountain climber. The exercise was performed without any warmup activity so that subjects underwent intense cardiovascular exertion to elevate their heart rate. Although the extent of exercise required for exertion is heavily dependent on interpersonal variations in fitness, approximately one minute of this exercise was found to induce a sufficiently elevated heart rate. The second measurement process was started immediately after the subjects chose to end the exercise. Data was collected with the subject lying in the supine position. The results of this study were used in section 7.1 to correlate fiducial points to blood pressure. However, this method contained discretized blood pressure measurements and could only be used as a first pass towards accurate predictions.

### 3.2 VCG Biopac

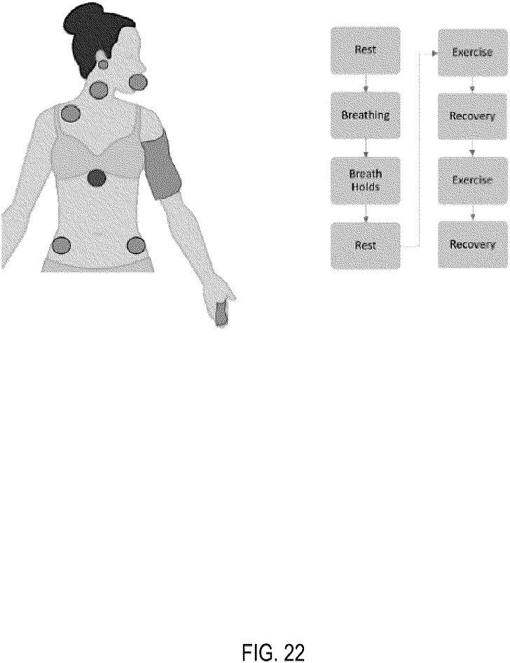

Experimental data for blood pressure estimation was collected at McGill University. The set up consisted of a custom built VCG (described in section 2.2), an BIOPAC acquisition system, and a Keyence laser displacement sensor. The BIOPAC was used to record electrocardiography, impedance cardiography, spirometry, non-invasive blood pressure, and photoplysmography. The Keyence sensor tracked the orthogonal displacement of the IMU sensor. FIG. 22a) shows the location of the attached sensors. The study procedure was conducted with approval from the McGill Ethics Board and a summary is shown in FIG. 22 b), with a detailed description available in Appendix I: Testing Protocol. The study took approximately 90 minutes per subject. All tests were conducted in the supine position. Two simple, motionless tests in a relaxed state were used to get baseline measurements in a “best case” scenario. The breathing techniques and breath holds were used to assist in signal filtration from inspiration and volume. The recovery immediately following a short intense exercise provided an elevated and rapidly changing heart rate and a change in blood pressure. This was repeated as it had the highest probability for acquisition errors as discovered in the pilot trials.

FIG. 22: (a) Sensor Set up, and (b) Experimental procedure.

The BIOPAC system was recorded using their built-in AcqKnowledge software. This software and their clinically proven routines were used to derive the following metrics from the raw signals: systolic blood pressure, diastolic blood pressure, mean blood pressure, pulse pressure, respiratory volume, QRS intervals, heart rate, left ventricular ejection time, stroke volume, and cardiac output. These signals were then processed and exported to MATLAB so that they could be used for blood pressure estimation, derivation, and signal filtration. To achieve a statistically powerful result, the target sample size was 100 subjects. Currently there has been 64 participants in the study. The average metrics of the study population can be seen in Table 1. However, the recruitment of additional subject has been put on to allow the study team to focus on the analysis.

TABLE 1

Study Population

| | |
|---|---|
| Description | Value |
| Participants | 64 |
| Percent Male | 57% |
| Age | 24.6 ± 4.4 years |
| Weight | 70.5 ± 16.4 kg |
| Height | 172.3 ± 10.6 cm |

### 3.3 Sensor Placement

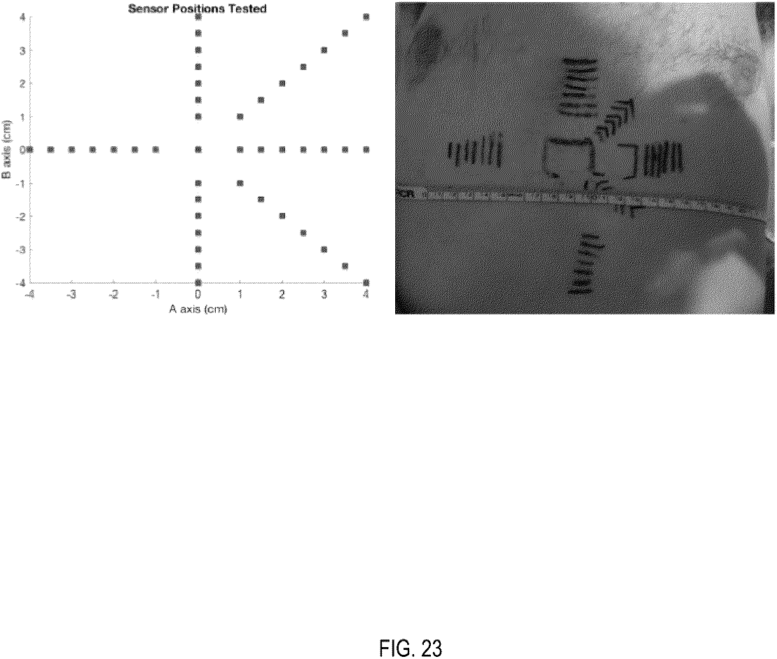

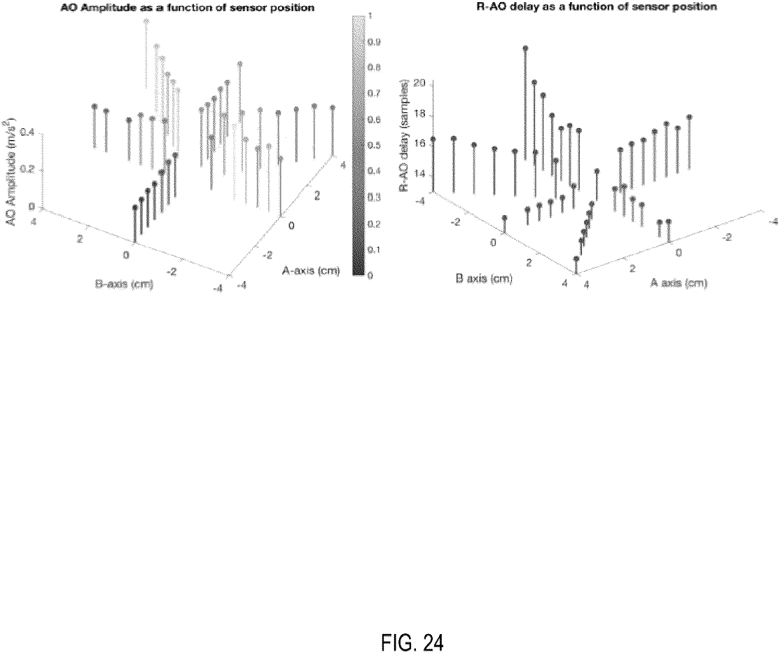

As discovered while completing the previous trials, sensor placement consistently produced a recurring difficulty during the test set up. The output waveform, mainly the locations and amplitudes of the fiducial points varies with placement on the chest. Therefore, this dependency must be developed by analyzing the waveform across each position. A first study was conducted to help design a more robust trial. The test consisted of one male subject with 42 positions tested for 5 minutes each. The coordinates of the locations are shown in FIG. 23. The experiment used two concurrent IMU/Arduino systems with one sensor fixed at the xiphoid process of the sternum as reference and the other varied around the chest. An analog ECG board was connected to the body with the output split to each Arduino. This provided a global timing system for synchronization between the two Arduinos. The results of this experiment are in section 4.1. The next steps are to develop a shorter routine as this took approximately 6 hours with one subject. We plan in the near future to expand the study to approximately 5-10 subjects to see if any concrete trends can be discovered.

### 3.4 Motion Capture

In a practical situation, it is unlikely for a user to be supine and motionless. As VCG records motion of the chest, additional motions pertaining to movement, walking, and voice present the opportunity to corrupt the signal. Therefore, a series of tests were conducted to characterize the functionality of the developed methods and to create new algorithms for motion detection, reduction, or elimination. The experiment took place at the McGill Center for Interdisciplinary Research in Music Media and Technology (CIRMMT) in the motion capture laboratory. VCG was recorded using the system described in section 2.2.2 with a BIOPAC MP160 used to record ECG as a reference. The movements were captured with a 16 infrared Qualisys motion cameras. An analog clock generated from the BIOPAC was used to synchronize between the three systems. Reflective markers were placed across the body, including points of interests such as the IMU, chest, and feet. Five subjects performed 9 activities, varying in orientation and level of motion artifact. The first recording was a traditional SCG recording where the subject was supine and motionless. The next two were motionless but lying on each side. Then a motionless test while sitting and while standing were recorded. A small level of motion artifact was added to a sitting test where the subjected moved.

FIG. 23: sensors placement grid and traced positions on the chest, with positive B oriented towards the head and positive A oriented to the left on the body their torso while remaining seated. The same level of motion was added to the next standing test where the subjected moved their torso while remaining standing. The final two tests consisted of walking. Since the motion capture cameras can only capture a specific range, the subject walked in a clockwise circle with varying speed and intensity of the steps. This was repeated with the subject walking in a counter-clockwise circle. The motion capture facility allowed us to quantify the timing, location, and intensity of motion artifacts, particularly foot falls for development of artifact removal algorithms as described in section 4.4 to enable real world use of the VCG sensor.

### 4. Signal Filtration

The typical morphology of a VCG signal shows high inter-subject variability yet little inter-beat variability. As a result, once the SCG signal is identified for a single subject, changes in the signal morphology can be attributed to changes in physiological activity. Similar to the transfer of blood pressure through the arteries, VCG waves traveling through the thorax are modulated during propagation. The signal undergoes frequency-dependent dispersion and attenuation due to the dynamically varying material properties of the thorax before reaching the sensor. The main causes of the modulation of VCG waveform morphology are sensor placement and respiratory activity. For example, the porosity of the chest decreases with respiration volume (RV), which dampens VCG amplitudes. The material properties along the path of propagation between the heart and the sensor can change drastically due to the complex architecture of the ribcage. These effects must be filtered from the signal in order to amplify its relationship with the mechanical operation of hydraulic cardiac valves.

### 4.1 Sensor Misplacement

The surface recording of vibrational waveforms are highly dependent on sensor positioning. The origin of cardiac vibrations begins within the chest at the heart. As the waves propagate to the surface, the recording position will impact the signal due to the directionality of a vibrational wave and the non-uniformity of the chest. The general technique of seismocardiography is to place the accelerometer at the xiphoid process of the sternum. However, some researchers use the upper sternum or the 5thintercostal space at the midclavicular line or others. Each of these positions will produce different waveforms which have their strengths and weaknesses. Considering the xiphoid process, there is still user error. As the human body does not have a perfectly reproducible shape, attaching the sensor to this position may be challenging to an inexperienced user. Even during our tests with a trained technician, we can observe varying VCG waveforms while attaching the sensor near the sternum.

A pilot study was conducted to investigate the effect of sensor misplacement. The procedures are outlined in section 3.4, where a single subject was measured at multiple positions of the sensor on the chest. For example, the limited results demonstrate that the AO point is weaker, and takes longer to reach the surface as the placement of the sensor is shifted further from the heart, as seen in FIG. 24 below.

This study provides evidence of the physical principals underlying vibrational wave propagation. However, due to the non-uniformity of the human body, there is a large amount of noise in the data. Therefore, in order to get a better idea of how the position affects VCG readings, we must repeat this experiment on more subjects before we can dive deeper into an analysis that will provide a quantitative mapping regarding placement and SCG amplitudes. Since this experiment is very time consuming per subject, we aim to test approximately 5-10 subjects to give us a base on the analysis and where to place the sensor for best results in our application. Once the sensor has been placed in an optimum position, there is still variation seen in the VCG waveform which can be related to respiration, exertion or motion artifact.

FIG. 24: Change in AO amplitude and timing. Note that the axes are rotated between graphs to offer both perspectives.

### 4.2 Respiration

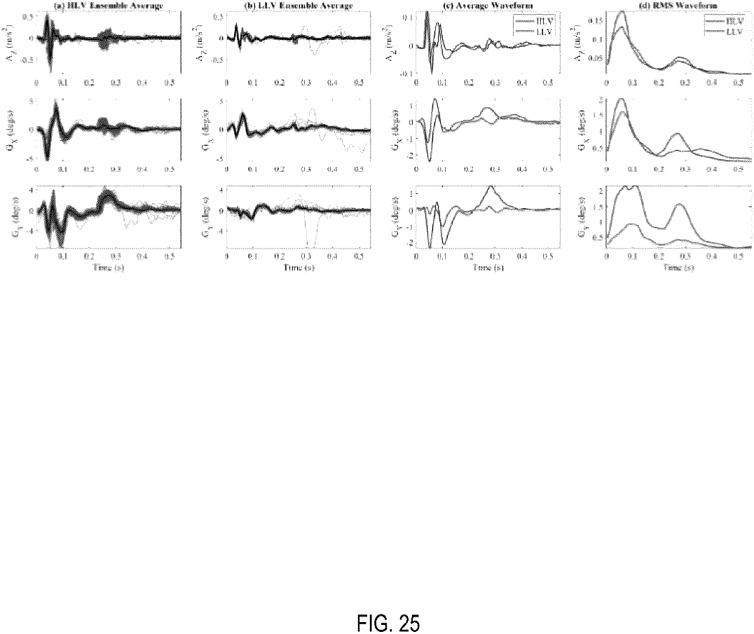

Following a precise positioning, the VCG waveform experiences modulation that is uncorrelated with changes in blood pressure. There are many physiological aspects which these can be attributed towards, however the most apparent is respiration. Respiratory modulation is common across most forms of physiological signals. Its effects can generally be attributed to three avenues: baseline wandering, frequency modulation, and amplitude modulation. For VCG, as the chest rises and falls with each breath, the sensor’s position relative to gravity’s acceleration changes and thus the baseline acceleration changes. Depending on the position of the sensor and shape of the person’s chest, this can be seen in all 3 axes. Inhalation, as an active process, produces a change in heartrate due to the nervous system. During inhalation, the body increases its heart rate in order to provide more blood for the muscles within the chest. This effect has been seen in other physiological signals, such as ECG, where RR interval is shortened. Presumably, the same effects will happen within the VCG domain per each beat. However, what is not clear is how the timing within each beat changes with respect to inhalation. The relationship between points and inhalation therefore needs to be characterized. The final major effect is a change in the amplitude of the VCG signal which is induced by respiration. In our previous paper, we demonstrated that a change in average RMS amplitude corresponds to a change in lung volume, instead of to respiration phase. FIG. 25 below shows these effects as published in that paper. It is still unclear of the physical reasonings behind the discoveries. There have been several theories which include the change in density of the chest, the change in intrapleural pressure, or change in cardiac output.

This was a preliminary study and worked on an average beat; this study will be expanded to study the effects on each beat and each fiducial point in order to characterize the effects due to volume. Additionally, this study was conducted during static breath holds and has been expanded to dynamic breathing exercises by using spirometer measurements to increase the range of known lung volumes. The experimental procedure is outlined in section 3.2, where we have accumulated enough data to begin the analysis on this section.

### FIG. 25: Wave Form Changes Due to (a) High Lung Volume, (b) Low Lung Volume, (c) Across all Subjects, (d) RMS For All Subjects

### 4.3 Exertion

A second major physiological contributor to signal modulation is exertion state. When the body becomes exerted and is spending more energy, the heart reacts to pump blood faster to the rest of the body. When the heart beats faster, it circulates blood more quickly by pumping smaller stroke volumes (SV) at quicker beat to beat (BTB) intervals (so the BTB and LVET is lower too). As soon as the exercise stops, less circulation is required and so HR and BP reduces. As the HR lowers further, the filling time is sufficiently increased (depending on the myocardial contractility of the heart) so that larger SVs may be pumped. However, larger SVs require a larger BP and so the BP rises as HR is reduced. Therefore, there is a nonlinear relationship between BP, SV, LVET, and BTB demonstrates high coupling between these measurements.

These effects have been observed in VCG morphology. Notably, cardiac time intervals are compressed in proportion to a lower BTB interval while the LVET fraction of each interval is relatively constant. Additionally, we notice a large increase in signal amplitude immediately after exercise followed by a rapid decay in amplitude and heart rate as the body recovers. However, in blood pressure, we do not see the exact same increase followed by an exponential decay and therefore this must be taken into account to produce a clean mapping between VCG and BP.

### 4.4 Motion Artifact

The final step in filtration is to filter for motion artifact. One of the major drawbacks of wearable sensors are their susceptibility to motion artifacts. As VCG consists of recording motions of the chest wall, all other motions could corrupt a signal and produce faulty results to algorithms and interpretations. Motion artifact can arise from a variety of sources such as body movement, footsteps, and sounds from inside the body such as voice, stomach, or ventilation. The experiments outlined in section 3.4 focus on motion artifact pertaining to body movement and walking. Detecting and removing these ailments are essential for any real-world implementation of a VCG system as users won’t always be supine and motionless. However, in a best-case lab environment, these effects can be assumed to be low as the supervised subjects remain relatively quiet and still. Current data was inspected manually, and unidentifiable signals were discarded. However, when implemented in a real system, manual analysis is infeasible. A simple solution, implemented in compares acceleration to gyration data to reduce noise. On this principle, some noise will be rejected as it effects the orthogonal measurements differently. Large motion artifacts could still corrupt both signals and this method would fail. From the experiments outlined in section 3.4, using a dual sensor configuration (Section 2.2.2) can mitigate large artifacts. Since the second sensor is placed approximately in the same location on the back of the chest, the two sensors should more in tandem with large artifacts. Additionally, placing the sensor on the spine was did not sufficiently pick up VCG signals and therefore all movement it records can be related to motion artifact. The analysis from these experiments are still to be completed, with a likely target of submission to the EMBC conference. Using a dual sensor configuration adds minimal software and processing complexity but increases hardware and system complexity. In some applications, only a single IMU is used and therefore we cannot rely on the dual sensor configuration to remove all motion artifacts and algorithms will be needed to interpret the single-sensor data. While currently undeveloped, there are several methods to tackle motion artifact such as signal energy, directionality, or machine learning techniques. These methods will be explored in the coming semester once a solid foundation on best-case blood pressure estimation has been derived.

### 4.5 Conclusion

The major benefit to using and IMU to record biometric data is the wealth of information available from a single sensor. However, the disadvantage is that not all the information is relevant and some of it needs to be decoupled from the information we are trying to extract. The difficulty begins at the start of the experiments, when the sensor is attached. Attachment, when misplaced, can cause non-linear effects to the VCG signal, which requires better studies to characterize it. The next major hurdle is respiration, which is coupled physiologically and systematically to the VCG recordings where work is in progress to quantify the couplings. Exertion effects the signal similarly to respiration however in a resting scenario the effects are negligible. Finally, motion artifact detection algorithms are essential for a real-world implementation of the system.

### 5. Analytical Derivation of BP

Blood is circulated between the lungs and body through the heart. It is the transport mechanism that maintains the oxygenation and temperature of the body. Blood flow is a result of pressure differentials created by ventricular contractions inside the heart. Its directionality is regulated by one-way, hydraulic valves. The vibrations generated by cardiac motion are measurable at the sternum, however, the physiological path of the waves has not yet been analyzed. Studies using echocardiography have found that the opening and closing of cardiac valves coincide with the occurrence of high vibrational amplitudes at the sternum. Physical models have attributed these sternal vibrations to the pressure of the heart acting on the ribs during processes such as longitudinal ventricular contraction and the ejection of blood. This perspective confirms the coincidence of the valve movements with fiducial points of inflection in the vibrational waveform. For example, a peak in the acceleration waveform coinciding with the aortic valve opening (AO) event indicates a drastic increase in velocity as the valve opens. The mitral valve opening (MO) event coincides with similar oscillations in the waveform, albeit with a smaller amplitude because the heart exerts less pressure on the thoracic cavity when the ventricles are contracted. Such a direct influence of valvular events on the morphology of the signal suggests that vibrational waveforms, like phonocardiograms, exhibit a strong causal relationship with mechanical valve operation. Our work is therefore based on the assumption that valvular motion creates the main oscillatory features that are characteristic of VCG waveform morphology.

### 5.1 Cardiac Cycle

A cardiac cycle consists of ventricular contraction during atrial relaxation, followed by ventricular relaxation during atrial ejection. An R-peak marks the beginning of the systolic phase of the heart, at which point an electrical impulse causes the ventricles to contract and eject blood through aorta and pulmonary artery. Blood simultaneously returns from the body to passively refill the atria through the pulmonary veins and superior and inferior vena cavae. Once the atria are filled and the ventricles are emptied, the diastolic phase begins. This is a period of ventricular relaxation during which the atria refill the ventricles. Once the ventricles are filled, the cardiac cycle restarts. The total volume of blood pumped by the ventricles per minute is the cardiac output (CO), which indicates the rate of blood flow through the heart. Both sides of the heart are synchronized although the left side is larger because it circulates oxygenated blood from the lungs to the entire body. Hence, the following discussion will focus on the left ventricle in which blood flow is regulated by the aortic and mitral valves.

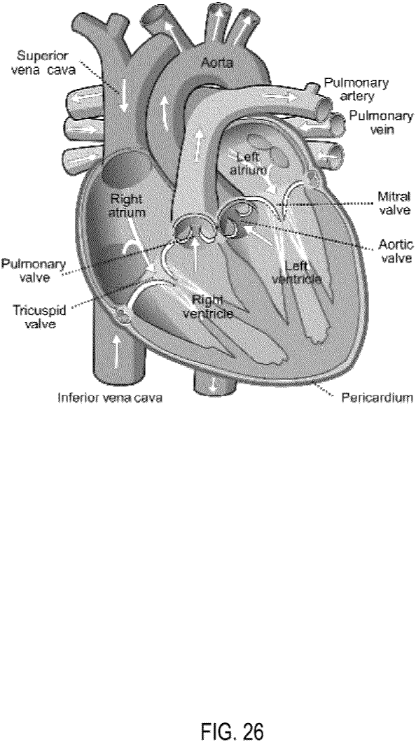

FIG. 26: Schematic of the heart indicating valves, ventricles, atria, and major blood vessels.

### 5.1.1 Aortic and Mitral Valves

The mitral valve (MV) regulates flow between the left atrium and the left ventricle. It opens when the left atrial pressure is higher than left ventricular pressure Pᵥₑₙ, which is a consequence of atrial contraction during the diastole. Conversely, it closes when Pᵥₑₙ is higher, which occurs during systolic ejection.

The aortic valve (AV) regulates flow between the left ventricle and the aorta. It opens when the left ventricular pressure Pᵥₑₙ is higher than the aortic pressure Pₒₐᵣ, which is a consequence of ventricular contraction during the systole. Conversely, it closes when Pₒₐᵣ is higher, which occurs during diastolic refilling.

The elasticity of cardiac valves allows for a passive, mechanical response to blood pressure differentials across them. The mitral closure (MC), aortic opening (AO), aortic closure (AC), and mitral opening (MO) valvular events provide fiducial markers in mechanical signals from which cardiac time intervals can be evaluated. Specifically, the MC and AO occur at the beginning and end of the isovolumic contraction period (IVCP) respectively, and the AC and MO define the isovolumic relaxation period (IVRP). After the MC event, ventricular pressure increases during the IVCP causing the AO event. The impulsive nature of valve movements suggests a strong contribution toward the sternal vibrations that coincide with the first heart sound. This correlation between valve operation and sternal vibrations has been further verified with echocardiography. The vibrational amplitudes of these fiducial points were therefore associated with cardiac muscle contractility and implicitly, the cardiac pressure cycle. In this manner, dynamically varying pressure levels in the heart directly stimulate valve movement, which specify the vibrational amplitudes that were detected at the sternum.

### 5.1.2 Cardiac-induced Vibrations

Cardiac pressure levels determine the hydraulic operation of valves in the cardiac cycle. As a valve opens, it compresses its surroundings and generates vibrational waves in the infrasound range. These waves traveled through the thorax and were recorded at the sternum as Vibrational Cardiography (VCG) signals. An IMU sensor was placed on the xiphoid process of the sternum with its Z-axis oriented outward along the dorsoventral axis of the body. Its exact position in reference to the sternum was indeterminable during testing. Six orthogonal motion signals were acquired from the sensor and filtered using a low-pass cut-off at 50 Hz. The linear a̅_{SCG} and rotational g̅_{GCG} vectors generated by cardiac activity were projected onto the coordinate axes as,

$${\vec{a}}_{S C G} = {a}_{x} \hat{x} + {a}_{y} \hat{y} + {a}_{z} \hat{z} , {\vec{g}}_{G C G} = {g}_{x} \hat{{θ}_{x}} + {g}_{y} \hat{{θ}_{y}} + {a}_{z} \hat{{θ}_{z}} z \qquad \text{(1)}$$

Which correspond to longitudinal and shear infrasonic waves. These a̅_{SCG} and g̅_{GCG} components were filtered from the IMU sensor signal as Seismocardiography (SCG) and Gyrocardiography (GCG) waveforms. The projection of the linear a̅_{SCG} onto the coordinate axes was retrieved using the equation,

$${\vec{a}}_{S C G} = \frac{{a}_{z}}{\left|{a}_{z}\right|} \sqrt{{a}_{X}^{2} + {a}_{Y}^{2} + {a}_{Z}^{2}} \qquad \text{(2)}$$

Using the known sensor orientation, the a_{Z} component was used as a surrogate for the direction of the a̅_{SCG} vector. A comparatively high oscillation amplitude was classified as an MC-AO complex if its frequency was within the experimentally verified range between 15-40 Hz, and if its occurrence was quasi-periodic. These large oscillations were enhanced by a VarWin function that compared the difference between amplitudes for all points within a sliding window. In this manner, the oscillations in the signal were transformed into peaks whose center occurred at t_{AO}, and slow-varying orientational changes or spikes from motion artifact were mostly filtered. Using the AO timestamps as a reference, the components of the AO vibration manifesting in the other axes were cross-verified to improve identification accuracy. A similar process enabled the classification of the AC-MO complex. However, due to a high variability in VCG morphology, individual valve events were relatively indistinguishable from the vibration corresponding to each heart sound. Regarding the MC-AO complex, the occurrence of the ECG R-peak was used as a surrogate for the MC point since tᵣ ≈ t_{MC.} The cardiac time intervals that were either measured or estimated from the VCG signal were,

$$\begin{matrix}{T}_{S y s} = {t}_{A C} - {t}_{M C - 1} , {T}_{D i a} = {t}_{M C} = {t}_{A C} \\ {T}_{B T B} = {T}_{S y s} + {T}_{D i a} = {t}_{A O} - {t}_{A O - 1} , {t}_{P E P} = {t}_{A O} - {t}_{r} \approx {t}_{A O} - {t}_{M C - 1} , \\ {t}_{L V E T} = {t}_{A C} - {t}_{A O}\end{matrix} \qquad \text{(3)}$$

Here, T_{sys} and T_{Dia} are the durations of the systolic and diastolic phases of the cardiac cycle, T_{BTB} is the beat-to-beat interval, T_{PEP} is the pre-ejection period, T_{LVET} is the left-ventricular ejection time (LVET) and a subscript of -1 corresponds to an event occurring in the previous cardiac cycle. Since cardiac valve operation was dictated exclusively by blood pressure differentials, valve-related events provided clear indicators of pressure crossovers in the heart. Additionally, the vibrational amplitudes have been found to increase with heart rate and other features in the VCG signal have been found to correlate with BP and maximal oxygen consumption. We therefore hypothesize that the cardiac blood pressure cycle can be derived from VCG signal morphology via its direct relationship with cardiac valve movements. Based on this, the following sections describe our development of a novel algorithm to calculate central, aortic BP from experimental VCG waveforms. Our method draws on principles in cuff-less BP monitoring including pulse wave analysis, multivariate regression, and oscillometry. It draws on techniques in pulse wave velocity although the typical measurements of Pulse Arrival Time (PAT) and Pulse Transit Time (PTT) are not directly used due to their low correlation with pressure levels.

### 5.2 Modeling Pressure-Induced VCG

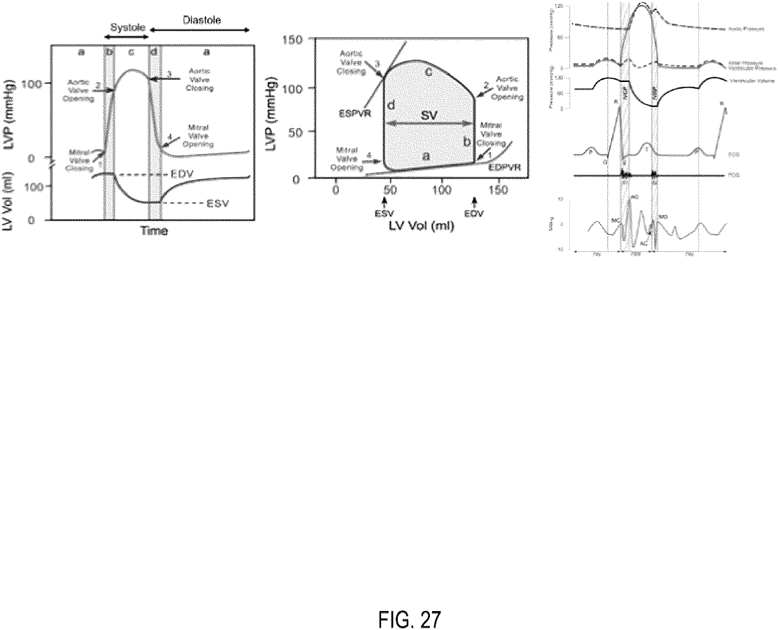

The dynamics of the cardiac cycle are regulated by a coupled pressure-volume relationship maintained in the chambers of the heart. During the systole, ventricular contractions induce changes in causing the ejection of blood through the AV and a corresponding decrease of the ventricular volume Vᵥₑₙ. As shown in FIG. 27(a), the ventricular volume is refilled during the diastole. This relationship between Pᵥₑₙ and Vᵥₑₙ during a cardiac cycle is mapped in the P-V loop of FIG. 27(b). Its manifestation in cardiac measurements is represented by the Wiggers diagram in FIG. 27(c). Note that the relationship between ventricular and aortic pressure is also indicated in the diagram and can be explained as follows. During the pre-ejection period T_{PEP} (after t_{MC-1}), cardiac muscle undergoes isovolumic contraction causing a drastic increase in ventricular pressure Pᵥₑₙ. At this time, the central aortic pressure Pₐₒᵣ is assumed to match the diastolic pressure level P_{Dia.} Once Pᵥₑₙ rises beyond Pₐₒᵣ at the time t_{AO}, the AV opens and blood is ejected into the aorta, resulting in an increase in P_{aor.} This hydraulically induced valve movement generates a mechanical force on the surroundings, causing vibrations to diffuse through the chest and be detected at the sternum. The aortic pressure Pₒₐᵣ continues to rise as it follows Pᵥₑₙ until the middle of the systolic phase when it reaches its maximum P_{Sys}.

A model describing the relationship between cardiac pressure and VCG waveform morphology must therefore explain how (i) the ventricular pressure cycle produces (ii) the central aortic pressure cycle through (iii) the mechanical movement of hydraulic valves which generate (iv) vibrational waves propagating through the chest.

FIG. 27: The cardiac pressure cycle showing (a) typical changes in ventricular pressure and volume, (b) the P-V loop representing a cardiac cycle and (c) Wiggers diagram displaying synchronized changes in pressure, volume, ECG, PCG, and SCG.

### 5.2.1 Central Aortic Blood Pressure

Central aortic pressure is normally between 90/60 and 120/80. Once the AV opens, the limited area of the AV restricts the flow of blood that is ejected from ventricular compression. As a result, Pᵥₑₙ continues to increase and is followed by Pₐₒᵣ until both equalize. Note that the maximum Pₐₒᵣ is recorded as systolic blood pressure P_{Sys}.

$${P}_{a o r , max} = {P}_{S y s}$$

It is possible to estimate how Pₐₒᵣ follows Pᵥₑₙby using the dimensions and material properties of the AV and aorta while accounting for vasodilation. This relationship is reflected in the pressure gradient across the AV, which is the difference in pressure between the ventricle and aortic root,

$$Δ {P}_{A V} = {P}_{v e n} - {P}_{a o r}$$

It determines the nature and intensity of the hydraulic, mechanical operation of the AV. This gradient has been found to follow a parabolic-shaped curve, which we have fitted to a third order polynomial with a zero slope at its trailing edge. It was used to derive Pₐₒᵣ and consequently, the systolic and diastolic pressure level.

$$Δ {P}_{A V} \left(t\right) = {q}_{1} {t}^{3} + {q}_{2} {t}^{2} + {q}_{3} t + {q}_{4} , t = t - {t}_{A O}$$

During the IVCP, the AV remains closed and Pᵥₑₙ increases drastically. At the AO event, the difference in pressure is given by the threshold pressure required to open the AV,

$$Δ {P}_{A V} \left({t}_{A O}\right) = {q}_{4} = {P}_{A V , t h}$$

The change in aortic pressure immediately after the AO event was assumed to be negligible, causing the changing pressure gradient to be determined solely by ventricular pressure,

$${\left(\frac{d Δ {P}_{a v}}{d t}\right)}_{t = {t}_{A O}} = 3 {q}_{1} {t}^{2} + 2 {q}_{2} t + q \_ 3 \approx {\left(\frac{d {P}_{v e n}}{d t}\right)}_{t = {t}_{A O}}$$

$$∴ {q}_{3} = {p}_{2}$$

At the end of the systolic phase at the AC, the AV closes when there is negligible flow due to a stabilized pressure equilibrium across the valve,

$$Δ {P}_{A V} \left({t}_{A C}\right) = 0 = Δ {P}_{A V} \left({T}_{L V E T}\right)$$

$$∴ {q}_{1} {T}_{L V E T}^{3} = - {q}_{2} {T}_{L V E T}^{2} - {p}_{2} {T}_{L V E T} - {P}_{A V , t h}$$

$$∴ {q}_{1} = - \frac{{q}_{2} {T}_{L V E T}^{2} + {p}^{2} {T}_{L V E T} + {P}_{A V , t h}}{{T}_{L V E T}^{3}}$$

At this point, the pressure gradient is no longer changing either,

$$\frac{d Δ {P}_{A V}}{d t} = 3 {q}_{1} {T}_{L V E T}^{2} + 2 {q}_{2} {T}_{L V E T} + p 2 = 0$$

$$∴ {q}_{1} = - \frac{- 2 {q}_{2} {T}_{L V E T} - {p}_{2}}{3 {T}_{L V E T}^{2}}$$

Equating the two values of q₁, we get,

$$\frac{{q}_{2} {T}_{L V E T}^{2} + {p}_{2} {T}_{L V E T} + {P}_{A V , t h}}{{T}_{L V E T}^{3}} = \frac{2 {q}_{2} {T}_{L V E T} + {p}_{2}}{3 {T}_{L V E T}^{2}}$$

$$∴ 3 {q}_{2} {T}_{L V E T}^{2} + 3 {p}_{2} {T}_{L V E T} + 3 {P}_{A V , t h} = 2 {q}_{2} {T}_{L V E T}^{2} + {p}_{2} {T}_{L V E T}$$

$$∴ {q}_{2} = \frac{- 2 {p}_{2} {T}_{L V E T} - 3 {P}_{A V , t h}}{{T}_{L V E T}^{2}}$$

If the diastolic pressure could be determined in an initial calibration period, the measurements could be scaled accordingly.

However, this approach is flawed because it is not possible to ignore the initial differential pressure immediately after the AO event while also including P_{AV},ₜₕ in the derivation. It would therefore be necessary to model the changing pressure gradient as a 5th order polynomial or extract its physical relationship from the simulation results in section 6. Until these results are mature, a better approach might be required that does not over-constrain the problem, such as a piecewise function. However, as a preliminary investigation, this approach was also emulated in DSP code. As will be discussed in section 5.3.5, the results have motivated new lines of investigation into this approach. Once the accuracy of the fitted polynomial is improved, we will incorporate physical constraints to mold this fitted polynomial into a physically valid aortic pressure waveform.

### 5.2.2 Ventricular Pressure Cycle

The behaviour of ventricular pressure Pᵥₑₙ during the systole has been monitored using a combination of continuous-wave Doppler echocardiography and invasive catheterization. While the catheter provided accurate pressure measurements, it was found that a fluid-filled catheter system induced a delay in measurements that were accounted for by echocardiography. Nevertheless, both results indicated the parabolic nature of the Pᵥₑₙ curve. In this context, Pᵥₑₙ was modelled as a second order polynomial with coefficients p₁, p₂, and p₃ such that,

$${P}_{v e n} \left(t\right) = {p}_{1} {t}^{2} + {p}_{2} t + {p}_{3} , t = t - {t}_{A O} \qquad \text{(4)}$$

The starting point of the temporal variable was shifted to AO so that the coefficients were independent of the overall signal time. In this context, the coefficient values directly reflect the behaviour of ventricular pressure within each cardiac cycle. The following derivation utilizes boundary conditions in the CC to calculate the coefficients of the polynomial.

At the occurrence of the AO event, Pᵥₑₙ is higher than Pₐₒᵣ by a certain threshold P_{AV,th}, which is speculated by the elasticity of the valve. As the mechanical properties of the valve change less quickly, this threshold was assumed constant.

$$\begin{matrix}{P}_{v e n} \left({t}_{A O}\right) = {P}_{a o r} \left({t}_{A O}\right) + {P}_{A V , t h} \\ ∴ {p}_{3} = {P}_{D i a} + {P}_{A V , t h}\end{matrix} \qquad \text{(5)}$$

The change in ventricular pressure can be obtained by differentiating the polynomial with respect to time,

$$\frac{d {P}_{v e n}}{d t} = 2 {p}_{1} t + {p}_{2}$$

However, note that t = 0 at t_{AO} in which case p₂ represents the slope of the ventricular pressure graph,

$${p}_{2} = {\left(\frac{d {P}_{v e n}}{d t}\right)}_{t = {t}_{A O}}$$

The slope is zero at peak ventricular pressure, which occurs approximately in the middle of the systole,

$$\begin{matrix}\begin{matrix}∴ {t}_{m a x} = - \frac{{p}_{2}}{2 {p}_{1}} , & {t}_{m a x} \approx \frac{{t}_{A C} - {t}_{A O}}{2}\end{matrix} = \frac{{T}_{L V E T}}{2} \\ ∴ {p}_{2} = - 2 {p}_{1} {t}_{m a x}\end{matrix} \qquad \text{(6)}$$

The occurrence of the MC event marks the beginning of the systole and can be approximated by the occurrence of the ECG R-peak. It also marks the start of the isovolumic contraction period (IVCP) at which point Pᵥₑₙ is

$${P}_{v e n} \left({t}_{M C}\right) \approx {P}_{v e n} \left({t}_{R}\right) = {P}_{v e n} \left(- {T}_{P E P}\right) \approx 8 \text{mm Hg}$$

Substituting equations (5) and (6) in (4),

$$\begin{matrix}∴ {p}_{1} {\left(- {T}_{P E P}\right)}^{2} - 2 {p}_{1} {t}_{m a x} \left(- {T}_{P E P}\right) + {P}_{D i a} + {P}_{A V , t h} = 8 \\ ∴ {p}_{1} = \frac{8 - {P}_{D i a} - {P}_{A V , t h}}{{T}_{P E P} \left({T}_{P E P} + 2 {t}_{m a x}\right)}\end{matrix} \qquad \text{(7)}$$

$$∴ {\left(\frac{d {P}_{v e n}}{d t}\right)}_{t = {t}_{A O}} = {p}_{2} = \frac{{T}_{L V E T} \left({P}_{D i a} + {P}_{A V , t h} - 8\right)}{{T}_{P E P} \left({T}_{P E P} + 2 {t}_{m a x}\right)} \qquad \text{(8)}$$

Ventricular pressure can therefore be estimated from the VCG waveform by constraining the curve appropriately. However, this quadratic fit neither considers nor leverages the physics of the pressure-volume loop.

### 5.2.2.1 Ejection of Blood From the AV

Ventricular contractions increase Pᵥₑₙ to force open the AV and eject blood into the aorta, resulting in a corresponding reduction in ventricular volume Vᵥₑₙ. The velocity of blood AV flowing through the AV was assumed to match the velocity of blood ejected into the aorta. The pressure levels Pᵥₑₙ and Pₐₒᵣ were therefore related through Bernoulli’s equation,

$${P}_{v e n} + \frac{1}{2} ρ {v}_{v e n}^{2} = {P}_{a o r} + \frac{1}{2} ρ {v}_{A V}^{2}$$

Given that the density of blood is 1060 kg/m³, the pressure gradient across the valve is,

$$∴ Δ {P}_{A V} = {P}_{v e n} - {P}_{a o r} = 530 \left({v}_{A V}^{2} - {v}_{v e n}^{2}\right)$$

Converting this value to mmHg,

$$Δ {P}_{A V} = 3.9753 \left({v}_{A V}^{2} - {v}_{v e n}^{2}\right)$$

When the ejection velocity of the jet of blood through the AV is at its maximum, the equation is reduced to its simplified form which is valid for both the AV and MV,

$$Δ {P}_{A V , m a x} = 4 {v}_{A V , m a x}^{2} \qquad \text{(9)}$$

The blood flowing out of the left ventricle and into the AV is also related through the continuity equation,

$$ρ {A}_{v e n} {v}_{v e n} = ρ {A}_{A V} {v}_{A V}$$

$$∴ \frac{d {V}_{v e n} \left(t\right)}{d t} = {A}_{A V} \left(t\right) {v}_{A V} \left(t\right)$$

$$∴ {v}_{A V} \left(t\right) = \frac{1}{{A}_{A V} \left(t\right)} \frac{d {V}_{v e n} \left(t\right)}{d t}$$

This influence of the exponentially declining volume of blood in the ventricle on the ejection velocity at the AV directly describes how differential pressure is related to the declining volume at the maximal velocity v_{AV,max},

$$Δ {P}_{A V , m a x} = 4 {v}_{A V , m a x}^{2} = \frac{4}{{A}_{A V}^{2}} {\left(\frac{d {V}_{v e n} \left(t\right)}{d t}\right)}^{2} \qquad \text{(10)}$$

Assuming the differential pressure across the AV can be derived from the amplitude of a_{SCG}(t_{AO}) in section 5.2.3, and the maximum cross-sectional area of the AV A_{AV}(t) can be estimated as shown in section 5.2.3.1, this equation indicates the declining volume of the left ventricle. This decrease in volume of the left ventricle may be related to the displacement of the sternum measured by the sensor and could therefore provide validation of the calculations relating to BP. It can further be related to the stroke volume,

$${V}_{S V} = {V}_{v e n} \left({t}_{A O}\right) - {V}_{v e n} \left({t}_{A C}\right)$$

The stroke volume could also be estimated as a function of the body surface area (BSA). While the significance of this metric in the calculation of BP has not yet been determined, its direct connection with key indicators of BP suggests that it could prove useful.

### 5.2.3 Mechanical Operation of Hydraulic Valves

During the occurrence of the AO event, if Pᵥₑₙ did not rise beyond its value at t_{AO}, the elasticity of the AV would cause it to relax open instead of generating an impulse. This suggests that the rate of change of the pressure differential dΔP/dt is related to the speed at which the AV opens, and the vibrations caused by the AO event. The amplitudes α_{z}(t_{AO}), gₓ(t_{AO}), and g_{Y}(t_{AO})

could therefore be mapped to the upward slope of the ΔP_{AV} curve. Note that the dP/dt metric is normally an indicator of ventricular contractility.

The mechanical energy required to open a cardiac valve is generated by a difference in the blood pressure levels on either side of the valve. Across the AV, the pressure differential ΔP_{AV} between the ventricle and aorta generates a differential force,

$$Δ {P}_{A V} = {P}_{v e n} - {P}_{a o r} = \frac{{F}_{v e n}}{{A}_{A V}} - \frac{{F}_{a o r}}{{A}_{A V}} = \frac{Δ {F}_{A V}}{{A}_{A V}}$$

The cross-sectional area of the valve A_{AV} is calculated in section 5.2.3.1. This differential force ΔF_{AV} induces blood flow into the aorta, during which a fraction of the force is redirected laterally to hold open the valve. During the systole, this fraction is balanced by the force F_{┴} required to open the valve. The extent to which the valve opens depends on the pressure differential as well as the viscoelasticity of the valve wall, which reduces as the valve opens.

$${F}_{⊥} = {F}_{t h} {e}^{{k}_{2} {A}_{A V}}$$

Here Fₜₕ is the threshold force required to open the valve. The dynamic relationship between these two antagonistic forces determines the rate at which the valve opens. Beyond the threshold, this hydraulic, differential force opens the AV with a lateral force that compresses its surrounding medium. Some of the energy from this mechanical movement diffuses through the surrounding medium as vibrational waves. The compression force could be modelled by treating the myocardium as a psuedoelastic material. In summary, the hydraulic force gets redirected laterally to the sides of the valve as an impulse, which is exerted on its surroundings and propagates as a compression wave.

### 5.2.3.1 Cross-Sectional Area of AV

The cross-section of the AV was calculated from the maximum diameter of the aortic annulus, which was estimated from the body surface area (BSA) of the subject using the relationship,

$${d}_{a o r} = 1.06 + 0.82 {A}_{B S A} - 0.2 = 0.86 + 0.82 {A}_{B S A}$$

The area A_{BSA} was calculated using the Mosteller formula for all experimental results obtained using the Biopac. This diameter represented the largest opening of the AV during the systole, that is, the upper limit of the cross-sectional area AAV. Note that the factor 0.2 was included to account for the difference between the supra-aortic diameter and the aortic annulus since the relationship of the annulus with BSA was not directly given.

### 5.2.4 Vibrational Wave Propagation

The vibrational waves at the sternum retain their characteristic energy profile during propagation. As a result, the energy spectrum of the vibrations can be directly linked to the compressions caused by valvular motion. In this context, the energy of the AO event can be obtained from the kinetic energy in the vibrational signals,

$$\begin{matrix}{E}_{K} = {E}_{K , l i n} + {E}_{K , r o t} = \\ \frac{1}{2} m {v}^{2} + \frac{1}{2} I {ω}^{2} = \frac{1}{2} m \left({v}_{X}^{2} + {v}_{Y}^{2} + {v}_{Z}^{2}\right) + \frac{1}{2} I \left({g}_{X}^{2} + {g}_{Y}^{2} + {g}_{Z}^{2}\right)\end{matrix}$$

The mass m and moment of inertia / of the sternum can be assumed constant over the duration of a single test. Hence, any variations in the vibrational kinetic energy waveform are directly manifested in the six degrees of freedom measured by the IMU sensor. Three-dimensional angular velocity was measured directly by the IMU gyroscope. However, the signals corresponding to linear motion required integration because they were measured in units of acceleration. The linear velocity was therefore calculated by integrating the acceleration signal using trapezoidal integration,

$$v = {\int }_{{t}_{R}}^{{t}_{R} + {T}_{B T B}} a \left(t\right) d t = v \left(t\right) + c o n s t .$$

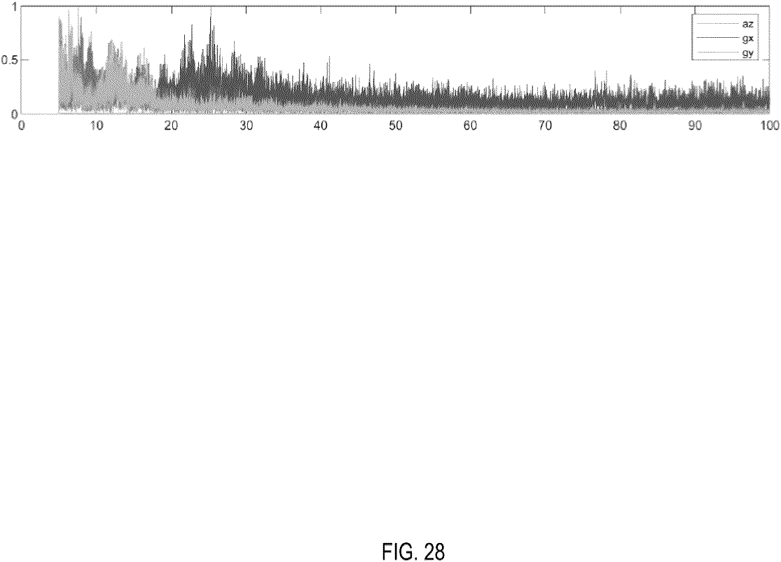

The velocity obtained by integrating the α_{SCG} vector was compared with a differentiated displacement signal from the Keyence sensor. This cross-verification between the IMU and laser sensor signals allows us to evaluate the fidelity of the integrated acceleration signal. The DSP code developed for this step is described in section 5.3.4. However, the two signals showed no correlation and the zero-crossing points of the reference velocity also did not coincide with observable fiducial points in the VCG waveform. Hence, the linear energy could not be obtained via integrating the time signal of the acceleration. Instead, its spectral profile shown in FIG. 28 could prove useful.

FIG. 28: Spectral profile of a VCG signal for the three main axes.

In the spectral profile of the signal, resonant peaks were observed along with their harmonics. Higher frequencies >18 Hz were attributed to valve operation while lower frequencies in the range 0.6-20 Hz were interpreted as ventricular contractions. The largest frequency component beyond 18 Hz was associated with the peak amplitude of the vibrational pulse generated by valve operation. The amplitude associated with the peak frequency above 18 Hz in each cardiac cycle was classified as the occurrence of the AO event. In a future step, this signal will be integrated in the frequency domain using its Fourier transform,

$${\int }_{- ∞}^{t} {a}_{S C G} \left(τ\right) d τ ↔ \frac{{F}_{{a}_{S C G}} \left(ω\right)}{j ω} + π {F}_{{a}_{S C G}} \left(0\right) δ \left(ω\right)$$

This will likely require separate processing or integration steps for each cardiac cycle. In this scenario, the frequency domain analysis could further be conducted over an ensembled average of the VCG signal generated from three cardiac cycles instead of one. The stability of the DSP code is expected to increase from the aggregation of three consecutive cardiac cycles. Once the energy profile of the vibrational wave is obtained, its modulation during propagation must be also be calculated. Despite retaining their characteristics, the vibrational pulses detected at the sternum undergo frequency dependent attenuation a(ƒ) and dispersion as they propagate a distance L through the viscoelastic medium of the thorax,

$$P \left(z + \text{L}\right) = P \left(z\right) {e}^{- α \left(f\right) L} , α \left(f\right) = {α}_{0} {f}^{η}$$

where f is the frequency component of the wave, P the pressure, and α₀ and ƞ are material parameters with α₀ ≥ 0 and 0 ≤ ƞ ≤ 2 for viscoelastic materials, and ≥ 1 for human tissue. These attenuation and dispersion transfer functions could be obtained by fitting experimental data from subject trials. For example, the influence of sensor placement and respiration volume on signal morphology could be detected by the corresponding changes that occur in its signal spectrum. This theory was confirmed by preliminary experimental results from the sensor placement study described in section 4.1. The pilot study revealed proportionate shifts in the frequency, phase, and amplitudes of resonant peaks in the signal spectrum as the sensor was placed further from the xiphoid process in specific directions. The development of these transfer functions is expected to be similar to the mathematical transformation developed to map the radial arterial pressure waveform up the artery to the central aortic waveform, which is also the principle of operation of our reference BP monitor.

### 5.3 Algorithm Development

The derivations in the previous section were developed alongside algorithms that roughly emulated the equations in the form of DSP code. Standard filtering techniques were not used on the raw signal to avoid any unwanted enhancement of the signal components. For example, frequency domain filters contain passband ripples that distort the spectrum of the filtered signal. These distortions could manifest in artifacts further along the processing stream and lead to unforeseen inaccuracies in measurement results. Once the measurement is finalized, the influence of filtering techniques could be further investigated. The main source of noise in the IMU signal for a supine subject was the respiratory component, which was also manually filtered. The following signal processing steps were conducted on the unfiltered signal.

### 5.3.1 Re-Sampling the VCG Signal

The VCG signal acquired by the sensor is sampled at a frequency of approximately 300 Hz even though the maximum frequency component of a VCG waveform was experimentally confirmed to be 50 Hz. This implied that the sampling frequency could be reduced to 100 Hz to obtain a significant speed boost with negligible loss in accuracy. As a safety precaution, the sampling frequency fₛ was set at 200 Hz. The IMU signal was linearly interpolated to match this frequency. This maintained a constant sampling frequency for all sensor signals processed by the code.

### 5.3.2 Respiration Filtering

The frequency spectra of all six axes were obtained using the Fast Fourier Transform (FFT) function. These spectra were normalized to eliminate variations in magnitude between the gyration and acceleration axes. The normalization also ensured that the highest frequency component in all six spectra had the same magnitude. A summation of these spectra within a 0-2 Hz range resulted in an amplified peak that was identified as the respiration rate (RR). Using this RR, a fourth order Savitsky-Golay filter was constructed with its frame length (in number of sampling points) given by,

$${n}_{S G} \geq \frac{{f}_{s}}{R R} , {n}_{S G} ∈ 1 , 3 , 5 , 7 , …$$

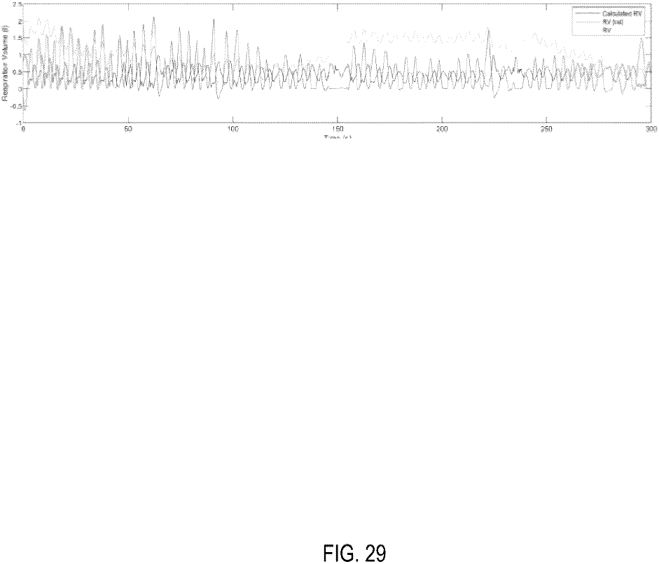

The respiratory component of each axis was extracted using this filter. Since respiratory activity manifested as slow oscillations in all six axes, the respiration signal was extracted by considering only those frequency components that were common to all three axes in the linear and angular domains separately. The mean frequency spectrum of the filtered respiratory signals over each set of three axes was obtained. The angle of the X axis was set as the angle of the acceleration spectrum whereas the angle of the gyration spectrum in the Y axis was set as the overall gyration angle. Both waveforms were shifted equally in either direction to compensate for phase delays between them. The average between both waveforms produced the estimation of respiration volume (RV) that is seen in FIG. 29.

FIG. 29: Respiration volume integrated directly from the spirometer (yellow), with resets (red), and calculated from the IMU sensor (blue).

Respiratory activity extracted using this method produced comparable results with the spirometer for most datasets. As is evident in FIG. 29, the reference measurement itself was unreliable. This is because the spirometer was designed to measure airflow at the mouth, which resulted in calculation inaccuracies once the signal was integrated and calibrated to RV. Additional inconsistencies were accrued between the sensor and spirometer since the sensor also detected chest movements and inhalation through the nose, which did not manifest in airflow measurements at the spirometer.

In the future, the phase mismatch between all six axes offers the greatest potential for improvement. A proper alignment of the delays between signal components could significantly boost the fidelity of the calculated RV waveform and possibly produce a measurement that is as accurate as the spirometer. Such a measurement would provide insights into the vectoral projection of respiratory motion on the detection axes of the sensor. This could enable differentiation between the motions originating the diaphragm versus the intercostal muscles and the results could further be verified by the breathing tests described in section 3. An accurate extraction of the respiration signal directly translates to an accurate filtration of respiration from the sensor signal, resulting in a higher fidelity VCG waveform. However, an active filtration technique will also be necessary to demodulate the effect of respiration volume on the oscillation amplitudes of VCG waveforms.

### 5.3.3 Classification of Cardiac Cycles

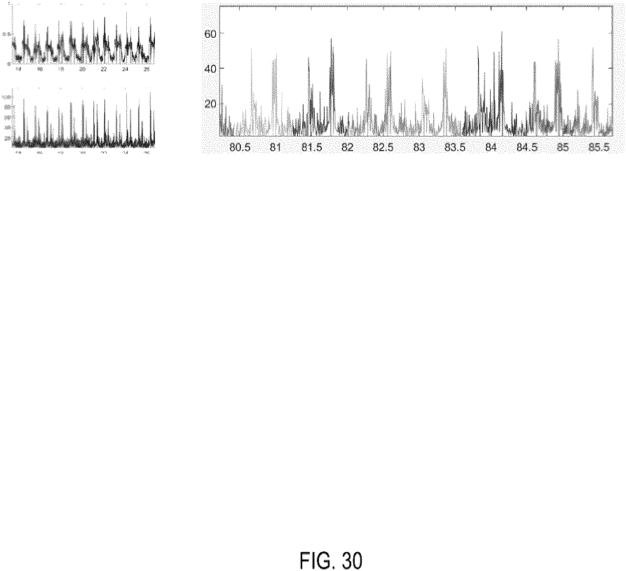

An accurate analysis of VCG morphology primarily depends on the identification of individual cardiac cycles within the signal. High vibrational amplitudes coinciding with the AO event are typically used as indicators of each cardiac cycle and can be cross verified using the quasi-periodicity in the VCG waveform between successive AO events. The oscillation amplitudes were previously amplified using the VarWin function which amplified large oscillation amplitudes by measuring the difference between points within a sliding window along the waveform. The VarWin function, however, required fine tuning for different axes and was limited to cleaner signals. The enhancement of the MC-AO complex required further improvement for noisy signals, such as those obtained in the comprehensive tests for BP analysis. As a result, the functionality of VarWin was extended to include the distance between points as well as their amplitudes. In this sense, the variations between points in a window were subsequently divided by the distance between them, resulting in the slope of the line connecting any two points. The VarWin function was therefore extended to a function called DerWin that calculated the maximum derivative within a sliding window. The output waveform consisted of a series of clearly identifiable Lorentzian peaks that coincided with the first and second heart sounds as shown in FIG. 30.

FIG. 30: (a) Comparison between the outputs of the VarWin (top) and DerWin (bottom) functions and (b) the DerWin output separated by cardiac cycles.

Note that the purpose of this work was not to identify cardiac beats but rather, to analyze them. In this context, cardiac cycles were separated by the R-peaks of the ECG signal because the ECG signal was less susceptible to MoArt and other noise. This choice of marker was reaffirmed by the fact that the timestamps of the R-peaks physically represented the beginning of a cardiac cycle. This separation of cardiac cycles also provided the input data necessary for the statistical analyses conducted in Section 7. Once separated, two Lorentzian peaks were fitted to either half of the DerWin output in each cardiac cycle, which were found to coincide with the MC-AO and AC-MO complexes. This could lead to an accurate calculation of LVET from the VCG waveform, which is estimated to produce a comparable accuracy with the LVET obtained from ICG. The amplitude and width of these peaks was investigated in calculations regarding BP with no conclusive results yet. The amplitudes displayed slowly varying oscillations corresponding to RV measurements. The nature of the widths has not yet been matched with any known physiological measurements. We speculate that a combination of the amplitude and width could be mapped to the vibrational energy at each event. Finally, the location of the peaks produced high accuracy HR and BTB measurements when compared with ECG, ICG, and NIBP references.

### 5.3.4 Extraction of Signal Energy

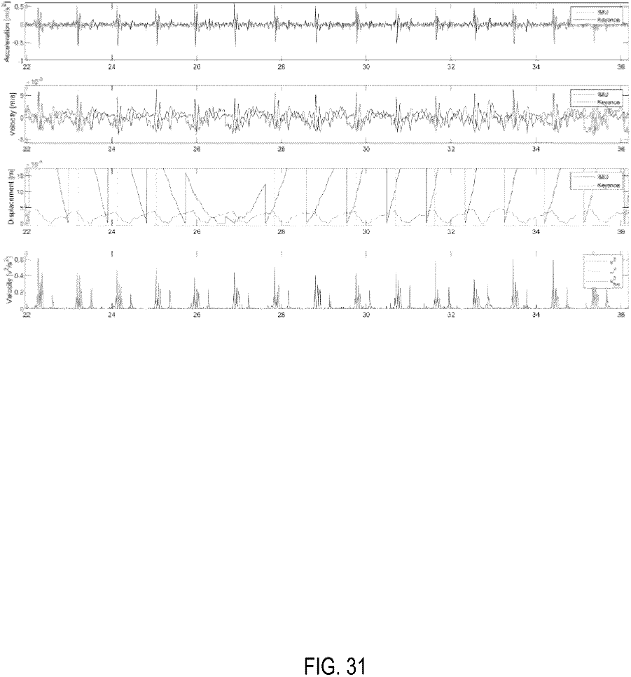

As explained in section 5.2.4, a calculation of the vibrational energy contained in the sensor signal for each valve event is essential to the derivation of BP from the VCG waveform. This process requires an accurate transformation of the linear acceleration signal to velocity. Improvements have been made regarding the identification of heart sounds via Lorentzians as well as in respiration filtering, however these improvements have not been enough. Acceleration signals acquired by the sensor do not yet produce velocity graphs that match those acquired by differentiating the displacement calculated for the sensor. This is likely due to the susceptibility of the sensor to interference from sources such as a high level of white noise, as well as the thickness of the tape that attaches the sensor to the sternum. Further processing is required to clean the sensor signal so that it produces a consistent velocity waveform such as that observed for the Keyence sensor in FIG. 31 (b). The consistency of the measurement further confirms its fidelity in measuring cardiac-induced sternal displacements.

FIG. 31: (a) Acceleration measured by the IMU compared with twice-differentiated displacement from the Keyence sensor, (b) Integrated acceleration from the IMU compared with differentiated displacement from the Keyence sensor, (c) twice-integrated acceleration from the IMU compared with the displacement measured by the Keyence sensor, and (d) The velocity-squared term of the vibrational Kinetic Energy detected by the (blue) IMU accelerometer, (red) IMU gyroscope, and (yellow) laser displacement sensor.

A promising outcome of this process was found in the ω² output of the angular energy from the gyrational gₓ and g_{Y} axes. The consistent Lorentzian-shaped waveforms resulting from this step display a high SNR corresponding to the first and second heart sounds. These waveforms could be fitted to increase the accuracy of cardiac beat detection in a similar manner to the DerWin output described in section 5.3.3.

The spectra corresponding to the three main vibrational axes is shown in FIG. 28. It shows that approaching this problem from the frequency domain might offer some promise. As seen in FIG. 28, the frequency distribution of the signal spectrum could offer a more consistent analysis and therefore be used to provide insights regarding the time domain signal. The phase profile of the signal is also yet to be investigated. Once resolved, the calculated linear velocity would enable a comprehensive analysis concerning the vibrational energy imparted by cardiac activity. Taken a step further, displacement calculations from the acceleration signal could be used to estimate either the change in diameter of the AV or the decrease in ventricular volume, which would provide data to support BP measurements using the equations presented in section 5.2.2.1.

### 5.3.5 Estimation of Blood Pressure

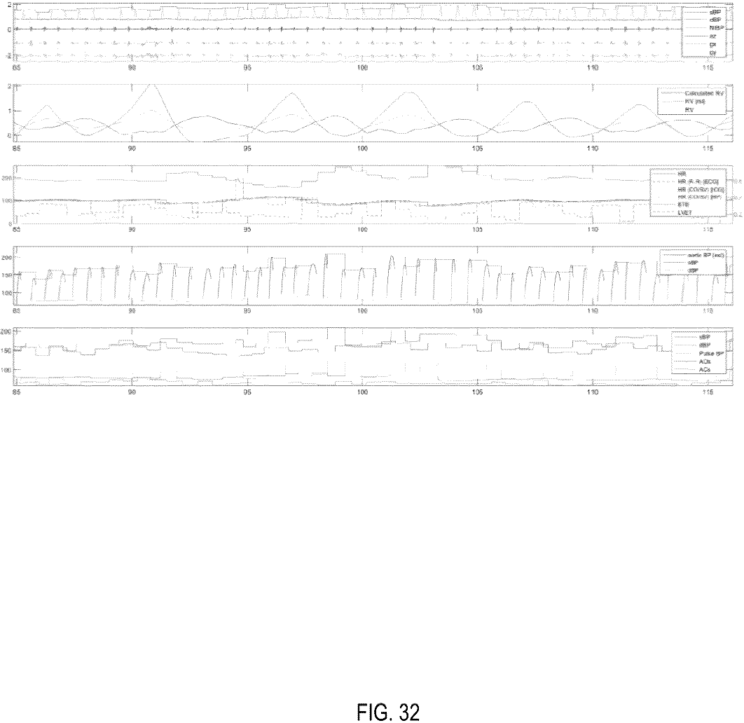

The objective of any clinical blood pressure monitor is to calculate the maxima and minima of the central, aortic pressure waveform for every cardiac cycle in real-time. Using mathematical transformations, the central aortic pressure waveform has been calculated from its corresponding radial arterial pressure waveform, which was acquired via oscillometric measurements on a finger. Once it was calibrated to a cuff sphygmomanometer measurement for a specific subject, this technique produced measurements of systolic and diastolic BP that were calibrated to the extremities of the radial waveform as shown in FIG. 32 (a). Similarly, a technique to map the VCG waveform to the central aortic waveform is being developed in this project report. While the previous discussions have built different approaches to address each aspect of the problem, a reverse engineering of the waveform was also attempted.

The central, aortic waveform followed a predictable path as discussed in section 5.2.1 in terms of its fitting parameters. Polynomial fitting was also experimentally investigated as seen in FIGS. 32 (d). First, the t_{AO} and t_{AC} timestamps were identified using the analysis described in section 5.3.3. This time period was identified as the systolic phase of the cardiac cycle for which the central aortic waveform needed to be generated. As a first attempt, it was modelled as a second order polynomial and fitted to the systolic and diastolic values of the corresponding radial waveform. The curve was fitted to three points in the time series corresponding to t_{AO} = 0, 0.52T_{LVET} and t_{AC} = T_{LVET}. The factor 0.52 was roughly estimated as the time at which the aortic pressure waveform reaches its maximum systolic level. The pressure at the third point at the end of the LVET was also estimated as 0.66% of the pulse pressure. While the parabolas appropriately fit the reference pressure waveform, their parameters do not yet match the polynomial derived in section 5.2.1. Further work is required to resolve this mismatch between polynomial fits. Once this issue is resolved, the accuracy of the fitted polynomials will be improved by incorporating physical constraints that mold this fitted polynomial into a physically valid aortic pressure waveform.

FIG. 32: Processed VCG signal for the different physiological metrics discussed. (a) NIBP and VCG waveforms, (b) RV derived from the spirometer and the IMU, (c) HR, BTB, and LVET calculations from the SCG, ECG, ICG, and NIBP signals. A higher error rate was observed for the ICG-based HR calculation. (d) Central aortic pressure waveforms fitted to the sBP and dBP measurements obtained from the NIBP during the systolic phase of each cardiac cycle. This represents the target measurement. (e) Calibrated pressure obtained by simply scaling the amplitudes of the SCG signal to match the first ten seconds of data. Note that the sBP and dBP waveforms in (a), (d) and (e) graphs are the same.

In the future, a template matching approach will also be investigated to analyze the VCG morphology of each cardiac cycle. The template will be constructed from an ensembled average of cardiac cycles over a sliding window of fixed duration (e.g. 10-100 seconds determined experimentally). In this approach, the first and second heart sounds of each new cardiac cycle would be aligned with the template by appropriately stretching/compressing new cycles to match their LVET. Since this template essentially represents the probability function of the signal, any variations in features of the time signal would be more pronounced when compared with the template and could therefore be utilized toward DSP analysis. For example, the vibrational amplitudes of the heart sounds could be compared with the template to obtain insights regarding interbeat variations in VCG morphology. Additionally, since the template would be constructed for a specific placement of the sensor, is expected that this approach could provide filtering effects for placement dependent changes in waveform morphology.

### 5.4 Conclusion

The four steps required to build a relationship between VCG waveforms and central aortic pressure are: (i) extracting specific vibrational waves from the sensor signal and mapping their propagation through the chest, (ii) modeling the cardiac motions responsible for their generation, (iii) deriving the hydraulic causes of the modeled mechanical motions, and (iv) calculating pressure levels from this hydraulic activity. The theory and DSP code required to build this relationship has made some progress since the analysis began in October, however, more analysis is required for these separate steps to converge toward a solution. Nevertheless, the results of the analysis are still promising by virtue of the fact that the individual analysis sections have grown to the extent that they are beginning to overlap with each other. As the analyses are further developed, relationships between individual sections will lead to a solution that explains the global connection between VCG and BP.

### 6. Simulation of VCG-Induced BP

To accurately predict blood pressure form VCG signals acquired at the chest, the complete cardiac cycle that occur and repeat with every heartbeat must be studied. Towards this goal, the COMSOL Multiphysics® software was used to accurately model sternal vibrations from the pressure differential at the heart valve. An electromechanical model of the heart was built to convert an ECG signal to expected electrical activity by applying potentials to an electroactive actuator. The resultant pressure differential due to contraction, ejected blood through an elastic, hydraulic valve opening, and allowed the vibrations to propagate to the sternum through a composite material representing the thorax. These vibrational waves were then probed in the form of a VCG signal. The simulation process was divided into the following sections for each of the above-mentioned steps. Section 6.1 describes an electrical activity model at the ventricle of the heart based on the principles of Dielectric elastomer actuators (DEA). An increase in the potential difference across the ventricle increases the pressure inside the chamber. Section 6.2 shows the deformation of a polymeric heart valve due to these pressure differentials. An increase in the pressure differential across the valve causes an increase in the flow rate of blood through the valve which results in a larger deformation of the valve walls. Finally, Section 6.3 discussed vibration propagation through the chest caused by these deformations.

### 6.1 Electrical Activity

Contraction and relaxation at the atrium and the ventricle of the heart is controlled by electrical impulses that are generated at the sinoatrial node. The electrical pulses propagate via flow of ions through the cardiac muscle cells. At the beginning of the impulse cycle, an influx of sodium ions inside the cell membrane causes the voltage across it to rise rapidly. At the peak of the voltage pulse, an outward flow of potassium ions and inflow of calcium ions causes calcium release from sarcoplasmic reticulum (SR) compartments in the cell. The increase in calcium results in muscle contraction by the sliding filament method. Due to the complexity in simulating such behaviour, the principle of DEA was used to model structural contraction from electrical activity. DEAs are comprised of electroactive materials as the dielectric between two compliant electrodes. When an external electric field is applied, the electrical energy is converted to mechanical energy which causes the electrodes to exert a force (Maxwell stress) onto the dielectric elastomer, causing a change in the dielectric size and shape. The following model describes this method.

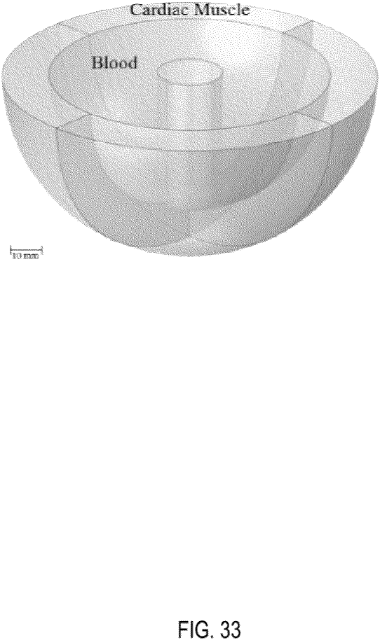

FIG. 33: Geometry of the proposed model to study electrical activity at the heart.

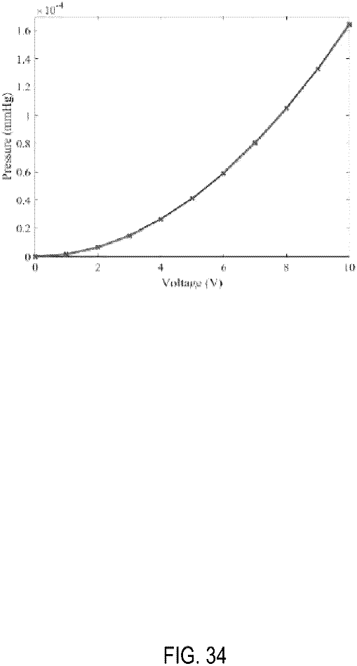

FIG. 34: A change in pressure caused by a potential difference at the ventricle.

The geometry of the proposed structure was built as a reference model based on the structure of the heart ventricles and can be found in FIG. 33. The outside membrane and the inner cylindrical layer of the structure contains the material properties of cardiac muscle whereas the material in between is filled with fluid containing the properties of blood. All material properties used in the simulation can be found in Table 2. Within COMSOL, structural mechanics module was coupled with the AC/DC module. A voltage was applied at the outside layer, while the boundary of the inner cylindrical layer was grounded. A fixed constraint was also set on this boundary so that the displacement for this section was zero in all directions. This was done to hold the entire model in place while the outside cardiac muscle layer deformed inwards. The Maxwell stress can be defined as,

$$σ = - \frac{1}{2} ε {ε}_{0} {E}^{2}$$

Where, ε₀ is vacuum permittivity, ε is the relative dielectric constant and E is the electric field. Hence, the force of contraction is directly proportional to the potential difference across the structure. Increasing the voltage difference across the structure resulted in an increase in pressure. A stationary study was performed for increasing voltages while the output was taken from the inside layer of the cardiac muscle. The result is shown in FIG. 34. This method allowed us to modify and set the pressure differential used in the following section to operate the valves.

### 6.2 Valve Operation Due to Pressure Differential

A cardiac cycle is divided into two major phases, ventricular contraction and relaxation. Deoxygenated blood flows to the right atrium from the vena cava while oxygenated blood flows to the left atrium from the pulmonary veins. Blood flows from the right atrium to the right ventricle through the tricuspid valve and from left atrium to the left ventricular through the mitral valve. Both valves close as a result of reversed pressure differential when the ventricles are filled, which produces the first heart sound S1. At this point the ventricles contract while the pulmonary and the aortic valves are still closed, increasing the pressure rapidly, resulting in isovolumetric contraction. As pressure in the ventricles increase further, the pressure differentials cause the pulmonic and the aortic valve to open resulting in rapid ejection. As ventricular pressure drops below the pressure in the pulmonary and the aorta, both valves close and produce the second heart sound S2. At this point, the ventricles start to relax, and ventricular pressure is decreased, resulting in isovolumetric relaxation. To study the valve opening and closing due to pressure differentials, the following simulation is performed.

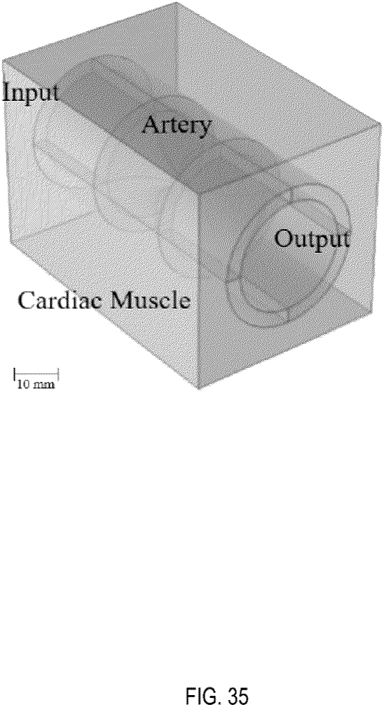

### FIG. 35: Geometry of the Proposed Valve Model

A simplified geometry approach was taken to simulate heart valves to reduce the complexity of the simulation while still maintaining acceptable results. FIG. 35 represents the geometry of the valve. The geometry consists of three domains: (i) A chamber for unidirectional blood flow through the Aortic Valve, (ii) A layer representing the valve walls, (iii) A layer of linear elastic material representing the cardiac muscle. All material properties used in the simulation can be found in

Table 5. The dimensions of the proposed polymeric valve were taken from the cross-sectional area of the aortic valve and further optimized through simulation. The material properties of the 3 domains correspond to: (i) blood, (ii) artery, and (iii) flesh. The structural mechanics module was used to couple solid mechanics and fluid flow using a Fluid-Structure Interaction (FSI) approach. A pressure differential from input to output was created through two user defined functions representing the pressure differential at the heart using MATLAB as shown in the FIG. 23. Blood was simulated as an incompressible fluid using the following Navier-Stokes equation.

$$\frac{ρ \left(\partial {u}_{f l u i d}\right)}{\partial t} + ρ \left({u}_{f l u i d} . ∇\right) {u}_{f l u i d} = - ∇ ρ + μ {∇}^{2} {u}_{f l u i d} + F$$

Where u_{ƒluid} is the velocity of the fluid, p is the pressure and F is any external forces on the fluid. The flow was deemed to be a single-phase laminar flow. Any backflow from output to input was suppressed in the simulation. Interaction between fluid and the surrounding structure was taken as one-way coupling, where pressure of the fluid loads on the structure, however, any deformation in the structure did not affect the fluid flow. Both laminar flow and one-way coupling were taken for simplicity and fast computational time. A time-dependent study was performed for the duration of a full cardiac cycle.

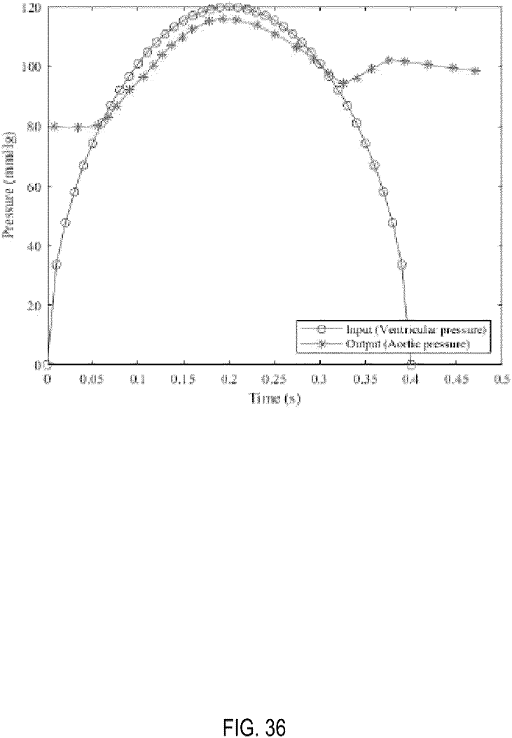

### FIG. 36: Pressure Differential at the Input and the Output of the Valve

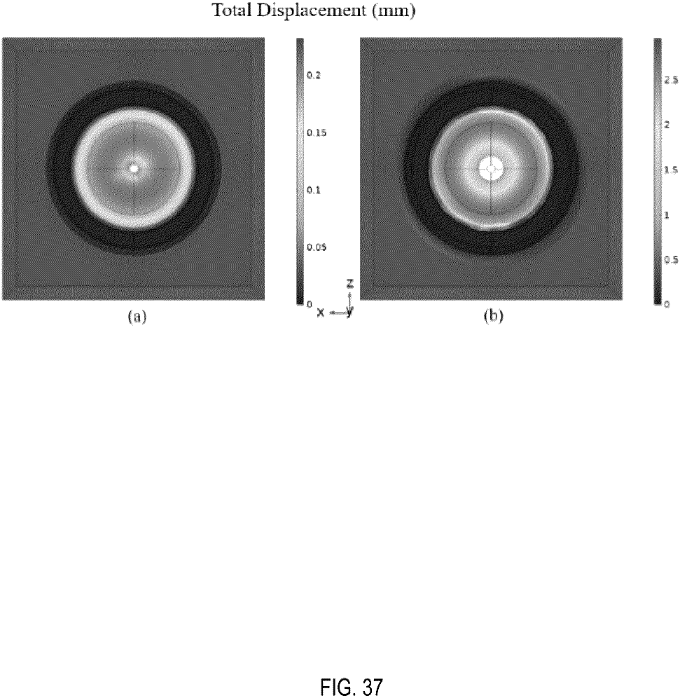

When the input pressure is higher than the output pressure across the valve as shown in FIG. 36, blood flows from input to output causing the valve to open. The deformation of the valve is shown in FIG. 37 below. These deformations were used in the following section to model sternal vibrations.

FIG. 37: (a) Deformation of the heart valve at 0.07 s (at the beginning when input pressure is higher than the output pressure). (b) Deformation at 0.21 s (when pressure differential between the input and the output is maximum).

### 6.3 Wave Propagation Due to Structural Deformation

Vibrations generated at the heart due to contraction and relaxation with each cardiac cycle can be recorded at the chest by an accelerometer and a gyroscope resulting in SCG and GCG waveforms. In order to connect these surface vibrational waves to the deformations at the heart valve, wave propagation due to structural deformation was studied in the following simulation.

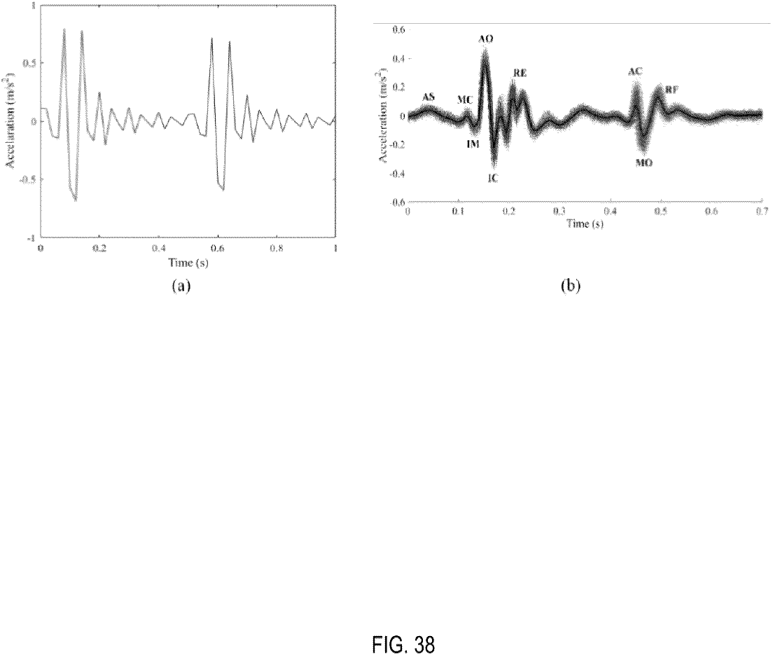

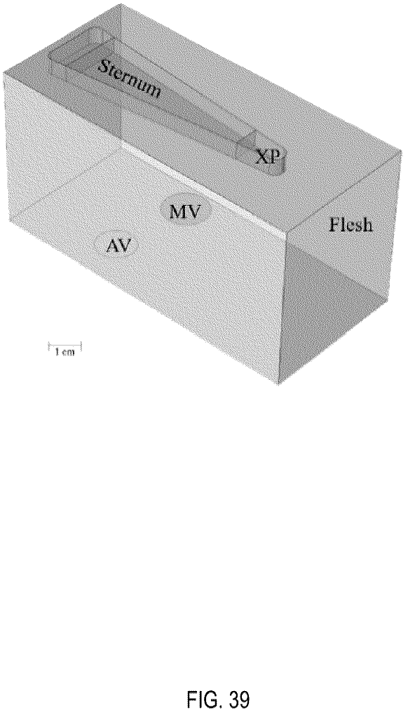

FIG. 25 represents the geometry of the proposed model. The geometry consists of 3 domains: (i) A sternum-like structure which was fixed at one end and the output was taken at the other end (Xiphoid Process), (ii) A homogenous linear elastic material representing the chest, and (iii) Two chambers for input deformation representing the two heart valves. The material for the three domains correspond to (i) bone, (ii) flesh, and (iii) cardiac muscle. Only solid mechanics within the structural mechanics module was used to simulate this model. A boundary load was applied at the aortic valve and the mitral valve sections at 0.1 s and 0.45 s representing time gap between the opening of the aortic and the mitral valve in a cardiac cycle. A low-reflecting boundary condition was used at the boundary of the flesh to reduce back reflections by absorbing all outgoing waves. The resultant oscillations due to the load were probed in the XP area in FIG. 39. The wave propagation was modeled via a time-dependent study covering a time interval of a full cardiac cycle. Output data was optimized and matched with experimental SCG data. FIG. 38 below shows the output of the simulation and compares the simulation result with experimental data.

Evident from FIG. 38, the resultant acceleration matches the experimental SCG waveform. Combination of the three sections mentioned above allowed us to isolate blood pressure from other physiological activity in the VCG signal.

FIG. 38: (a) Simulated acceleration at the XP compared to the (b) acceleration acquired through experiment.

### FIG. 39: Geometry of the Wave Propagation Model

### 6.4 Conclusion

The material parameters used in each of the section is shown in Table 2 below.

TABLE 2

Material Parameters

| Material | Young’s Modulus (MPa) | Density (Kg/m³) | Poisson’s ratio | Dynamic viscosity (Pa^{∗}s) | Relative permittivity |
|---|---|---|---|---|---|
| Cardiac Muscle | 180 | 1000 | 0.47 | | 50,000 |
| Blood | | 1060 | | 0.005 | 80.2 |
| Artery | 0.82 | 960 | 0.45 | | |
| Bone | 1500 | 1400 | 0.42 | | |
| Flesh | 0.85 | 900 | 0.43 | | |

For each section, a user defined mesh was implemented and optimized to minimize computational intensity. In areas of the structure where detailed calculations were unnecessary, a larger mesh size was used which decreased computational time with a negligible loss in accuracy.

While the three models in each section individually performed the assigned task, in future, a model combining all the sections will be built. Moreover, to build a more accurate representation of the heart, the following complexity will be introduced in the model in the future:

Introduce more geometrically accurate design of the heart chambers (aorta and ventricle), valves, chest and the sternum.

Design a more accurate representation of the chest including lungs to investigate the modulation of VCG by inhalation, exertion, HR, etc.

Model blood flow through the valves as turbulent using Reynolds-Averaged Navier-Stokes (RANS) approximation.

### 7. BP Estimation via Statistical Methods

A machine learning approach towards extracting BP from a VCG signal was also investigated. Machine learning can review large volumes of data and discover trends and patterns that would not be readily apparent to humans. Therefore, it could be useful in identifying correlations between our VCG signal and corresponding BP values that were overlooked during analysis or simulation. Specifically, the performances of classical regression approaches were compared with the performance of a more modern neural network (NN) approach. The classical regression approaches were chosen as a baseline and the NN approach was chosen because of the extraordinary success of NNs in industry and academia in recent years.

A machine learning approach towards extracting BP from a VCG signal was also investigated. Machine learning is often viewed as a “black-box” approach to problem solving since it is generally more results-oriented. Therefore, the goal here is not to analyse or understand the underlying biological processes that give rise to our acquired VCG signal, that is handled by our analytics and simulation. Instead the goal is to statistically model these processes, as accurately as possible, using machine learning techniques. The idea is that, in conjunction with our analysis and simulation work, we can build a comprehensive understanding of how vibrations travel from the heart to the sternum, and how they can be interpreted to calculate BP values.

Specifically, the performance of classical regression approaches was compared with the performance of a slightly more modern neural network approach.

### 7.1 Omron Correlations

A total of 50 datasets were examined in the study discussed in Section 3.1. Four out of the fifty data sets were discarded due to poor signal quality in either the vibrational or electrical waveforms. The failures were attributed to acquisition errors from sensor displacement or interruptions in the physical connections. Each dataset contained three discrete blood pressure measurements from the Omron S10 cuff monitor. The measurements were spaced one minute apart. The AO events in the α signal were identified using our custom algorithm and sensor as mentioned previously. Note that the identification algorithm uses both the a^{→}G and ^{→}GG components to classify heartbeats although the ^{→}GG component was not directly used in the blood pressure calculations that will be explained below. This is merely because it was simpler to focus our work on the linear component before including the rotational component.

The oscillation amplitude of an AO event, va_{z}(t_{AO}), was calculated as the peak of the waveform produced by the VarWin function at its corresponding timestamp. Consecutive vα_{z}(t_{AO}) values were averaged over the duration of the cuff deflation, which lasted approximately 30 seconds. The result was used to calculate the systolic blood pressure of a subject, P_{sys}, by using the equation,

$${P}_{s y s} = k \times v {a}_{z} {\left({t}_{A O}\right)}^{0.2}$$

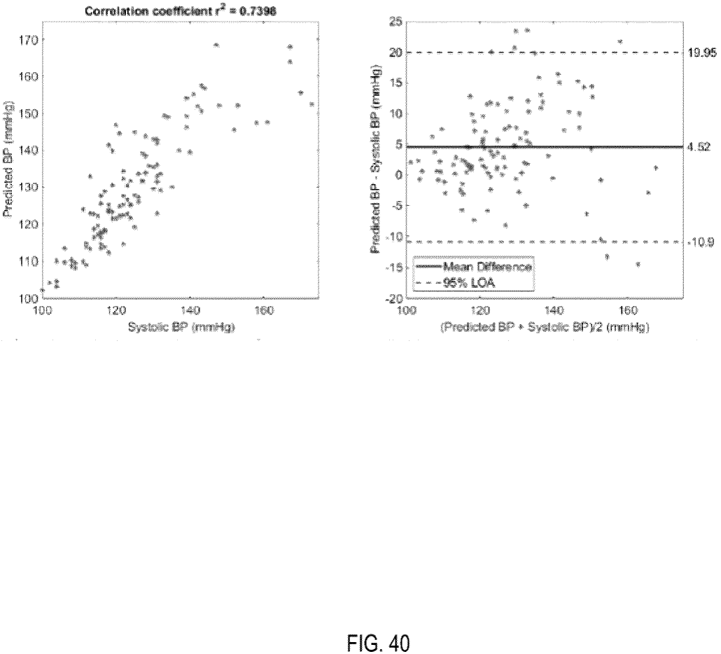

The scaling factor was obtained by calibrating the amplitude to the first blood pressure measurement obtained from the subject. This procedure is the same as the calibration of blood pressure for a finger cuff. In this manner, the peak oscillation amplitude of the SCG waveform was used as a primary indicator for blood pressure. FIG. 40 below shows the accuracy of this method when compared with reference cuff measurements.

FIG. 40: Correlation (left) and Bland-Altmann (right) plots of the measured systolic blood pressure in comparison with the calculated VarWin amplitude at the AO event for each subject. The calibration measurement was excluded from this comparison.

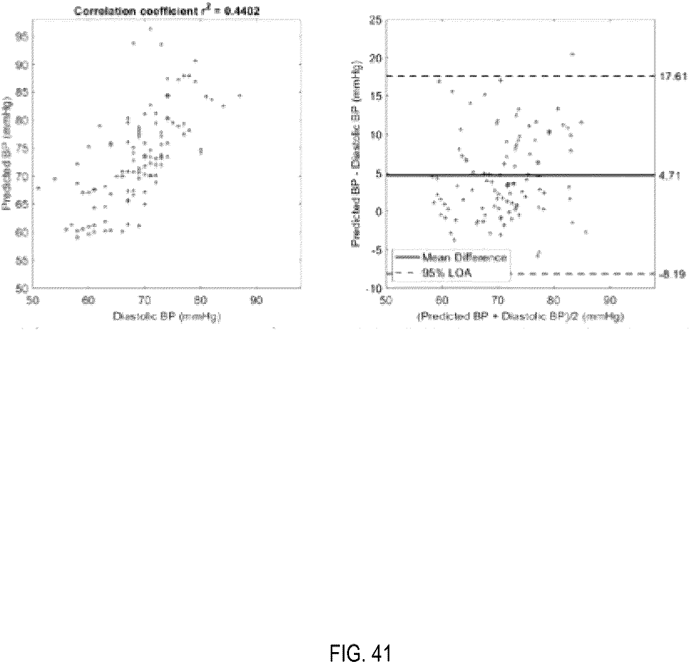

Hence, the SCG oscillation amplitude that coincides with the AO event was found to correspond with the maximum pulse pressure differential that is induced across it. The same calculations were performed for the AC-MO complex at the second heart sound in order to calculate diastolic blood pressure as shown in FIG. 41.

FIG. 41: Correlation (left) and Bland-Altmann (right) plots of the measured diastolic blood pressure in comparison with the calculated VarWin amplitude at the AC event for each subject. The calibration measurement was excluded from this comparison.

A strong correlation between measured systolic blood pressure and the calculated VarWin AO amplitudes confirms the potential of this method in calculating blood pressure. However, a relatively weaker correlation between diastolic blood pressure and the averaged VarWin AC amplitudes indicates that the method requires further development. This development is currently in progress. Key features of the new algorithm will include:
Derivation of the cardiac blood pressure cycle from vibrational wave analysis.
Inclusion of GCG data in BP calculations because a significant fraction of the vibrational energy is in the gyrational waveform.
Considerations for respiration volume, sensor placement, fitness, age, etc. as mentioned previously.

The direct statistical correlation approach was applied to the new data recorded in section 3.2 to the real time blood pressure readings. Now instead of BP readings per recording, there were hundreds (one per heartbeat). This was used to facilitate continuous BP estimation. However, the straight mapping form AO to NIBP did not produce meaningful correlations. This is hypothesized because of the large signal fluctuations seen in both the VCG and BP signals, in which they dependencies need to be filtered (Section 4). Given that these dependencies produce relationships outside of our realm of knowledge. It is difficult to perfectly remove all scenarios as it is likely we still do not know all possible relationships. Therefore, a more intelligent method must be used to derive blood pressure purely from a statistical method – machine learning. Using the machine learning techniques, we are able to incorporate thousands of possible dependencies into the relationship between the VCG waveform and the blood pressure signals. The methods which have been explored are outlined in sections 7.2 to 7.6.

### 7.2 Regression Analysis

The first step in a machine learning analysis is to use classical regression. Three algorithms were used in this investigation; a linear support vector regressor (SVR), a K-nearest neighbours regressor (KNN) and a random forest regressor (RF).

### 7.2.1 Support Vector Regression (SVR)

The Support Vector Regression (SVR) uses similar principles to those of the Support Vector Machine (SVM) for classification. In this method, a hyperplane is identified which maximizes the margin between support vectors (datapoints at the boundaries of each class) in the dataset. The difference employed in an SVR is that a margin of tolerance (epsilon) is set in approximation to the SVM in order to allow for regression analysis instead of finding a decision boundary for classification. SVR tends to work well with high dimensional data and is relatively memory efficient, but its performance tends to decline as datasets get larger.

### 7.2.2 KNN

The KNN algorithm uses “feature similarity” to predict values based on an input datapoint. Each input datapoint is assigned a value based on how closely it resembles the datapoints in the training set. The algorithm calculates the distance between the input point and each the training point (using a specified distance metric). The points taken into consideration are K training points with the nearest distance to the input point. In the case of regression, the prediction made by the algorithm is the average of all the labels of these K training points. Since predictions are made based on a comparison with the training set, no training time is required. This also makes it easier to add new data to the model, as it will not require re-training the entire model. However, KNN tends to perform poorly on high dimensional data or large datasets.

### 7.2.3 RF

The Random Forest algorithm ensembles multiple decision trees using a technique called bagging. Each decision tree is trained on a different sample of the training set and sampling is done with replacement. The motivation is that combining the predictions of multiple decision trees trained on slightly varying versions of the training set will result in more accurate and robust predictions than using a single decision tree. RF tends to perform well on large datasets and with high dimensional data, but it is prone to overfitting since decision trees are also prone to overfitting.

### 7.3 Data Preprocessing

To start, the raw Biopac and VCG data from our experimental testing phase (described in Section 3) were used to construct features and labels. A feature is a measurable property of the object being analyzed and a label is the value that object has. In supervised machine learning, features and labels are used to train a model, and that model is used to predict the labels of unseen data using the measurable features as input. In our case, the object is a cardiac cycle, each feature is the amplitude of the VCG signal at a certain point in time, and the labels are the discrete BP values.

### 7.3.1 Labels

The Biopac outputs a continuous BP wave, in which the peaks are systolic values and the troughs are diastolic values. Therefore, the labels were these systolic or diastolic BP readings.

### 7.3.2 Features

Our array of features was constructed as follows. First, the VCG signal (in each axis) for each subtest was separated into cardiac cycles (CCs) based on the onset of the P-wave in the ECG signal, giving a variable number of CCs (depending on the duration of the subtest being split). Each CC was resampled at a sampling frequency of 200 Hz, resulting in a uniform length of 500 elements per CC. These uniform-length CCs were then concatenated to form a preliminary n × m feature matrix, where n is the number of CCs in the dataset and m is the number of elements per CC (which in our case is 500).

$$\left[\begin{matrix}c {c}_{1} \left[0\right] & c {c}_{1} \left[1\right] & ⋯ & c {c}_{1} \left[m\right] \\ ⋮ & ⋮ & ⋱ & ⋮ \\ c {c}_{n} \left[0\right] & c {c}_{n} \left[1\right] & ⋯ & c {c}_{n} \left[m\right]\end{matrix}\right]$$

The above process is described for one single VCG axis. However, our IMU records data in 6 axes. These are the same coordinate axes described in Section 5.1.2; a_{X}, a_{Y}, a_{Z}, g_{X}, g_{Y} and g_{Z}. Therefore, it was possible to concatenate multiple axes (to give more features per CC), and we needed to determine the optimal combination of these axes. Since the IMU is place on the sternum of a subject with its Z-axis pointed outward along the dorsoventral axis, a majority of the vibrational energy from the VCG signal is oriented along the a component of linear acceleration as well as its orthogonally coupled g_{X} and g_{Y} components. Additionally, despite our hypothesis that the most important axes are the a_{Z}, g_{X} and g_{Y} axes, it was determined that all 6 axes should be included as a control. Therefore, the following axis combinations were chosen:
1. SCG: α_{z}
2. VCG: a_{z}, gₓ, g_{y}
3. All: aₓ, a_{y}, a_{z}, gₓ, g_{y}, g_{z}

Finally, the time series of the VCG data had to be taken into account. Since each CC was being resampled using a 200 Hz sampling frequency, we were losing all of the time series data for each beat (data which could prove to be very useful in extracting BP). Therefore, the time series for each VCG signal was split into CCs corresponding to the VCG signal itself. The initial idea was to concatenate the time series for each CC onto its corresponding CC (similar to how the axes were concatenated). However, since each CC had been resampled, the time series for each CC was nearly identical, the one difference between them being their gradients. Therefore, it was decided to append the gradient of each time series instead of the entire time series, leading to our final three feature combinations:
1. SCG: t, α_{z}
2. VCG: t, a_{z}, gₓ, g_{y}
3. All: t, aₓ, a_{y}, a_{z}, gₓ, g_{y}, g_{z}

### 7.3.3 Data Cleaning

During the feature construction phase, it was observed that many of the BP values were incorrect or NaN. This was either due to the continuous BP monitor recalibrating during a test, or the Biopac software misclassifying the BP peaks and troughs in post-processing. Therefore, some data cleaning needed to be performed.

To do this, a script was written that would loop through each subtest in a given subject folder, remove the obviously incorrect BP values (i.e. values that were NaNs) and plot both the BP values and the VCG signal. These plots were then inspected visually to determine at which ranges in the signal the BP values were unreliable. The unreliable ranges were removed from both the BP and VCG signals and also recorded in a text file.

### 7.4 Classical Approach

Once the features had been constructed and cleaned, they were used to train, fit and evaluate the three aforementioned regression models.

The evaluation was done through a circular 10-fold split of the data (training on 9 folds and testing on 1 each time) on one test subject, calculating the average correlation coefficient of the true and predicted BP values for all folds. In addition to the cross-validation-style evaluation method, a hyperparameter grid search was performed for each model to determine the combination of hyperparameters that gave the highest evaluation score. The results of the first model evaluation phase are shown in the Table 3 below.

TABLE 3

R2 scores of SVR, KNN and RF models when trained on and evaluated with data from one subject, using circular 10-fold cross validation

| Model | Best Hyperparameters | Correlation Coefficient | | |
|---|---|---|---|---|
| | | SCG | VCG | All |
| SVR | kernel = rbf | 0-2376 | 0.1737 | 0.2107 |
| | C = 100.0 | | | |
| | Epsilon = 0.0 | | | |
| KNN | n neighbours = 57 | 0.0890 | 0.3852 | 0.0716 |
| RF | max_features = sqrt | 0.0825 | 0.1096 | 0.0595 |
| | n_estimators = 200 | | | |
| | max_depth = 30 | | | |

Therefore, as shown in Table 3, the highest correlation coefficient observed between the predicted and true BP values was 0.3852, obtained with a KNN regressor using 57 nearest neighbours on the VCG feature combination.

### 7.5 Validation Against Respiration Volume

For the NN part of our statistical approach, a 1D convolutional neural network (CNN) was used.

### 7.5.1 Cnn

Convolutional neural networks (CNN) make use of convolving filters that are applied to local features. This concept is useful because forcing the extraction of local features ensures a certain degree of shift, scale and distortion invariance. Although they were originally proposed for computer vision tasks, CNN models and architectures have since been proven to be significantly effective many other tasks. A 1D CNN is essentially a CNN that uses 1D filters instead 2D filters. These models can be useful for analyzing time series data, hence why they were chosen.

### 7.5.2 Validation Step

Since there is little precedent in literature of 1D CNNs being used to analyse SCG data, a small validation step was taken before attempting to calculate BP values. This validation step consisted of a 1D CNN that was trained to predict the respiration volume state of a test subject based on the SCG signal. That is, given an SCG beat, determine whether that beat is from a period of high lung volume (HLV) or low lung volume (LLV). Although this task is not exceedingly similar to our main task (one is binary classification of lung volume while the other is non-binary regression of blood pressure), it was chosen as a validation step because it provided insight as to whether a 1D CNN could adequately capture the pertinent information in a given SCG signal.

To carry out this validation step, the SCG data from the HLV and LLV subtests of our experimental results were processed as described in the Data Preprocessing section to obtain features. For the labels, a “1” was assigned to HLV beats and a “0” to LLV beats.

The 1D CNN model used to classify lung volume had 2 convolutional layers, a max pooling layer, dropout regularization to improve generalization and a softmax activation function at the output for classification. The model was trained for 50 epochs with a sparse categorical cross-entropy loss function and the Adam optimizer. This model was evaluated on the test set of 401 samples and achieved an accuracy score of 89.5%. The confusion matrix is shown in Table 4.

TABLE 4

Confusion matrix for 1D CNN lung volume classification

| | Predicted 0 | Predicted 1 |
|---|---|---|
| Actual 0 | 115 | 22 |
| Actual 1 | 20 | 244 |

Therefore, the validation proved to be encouraging, as they showed that a 1D CNN has the capability to accurately interpret an SCG signal with reference to a simple binary classification task. Moreover, these results suggested that a 1D CNN might well be suitable for BP calculations.

### 7.6 Neural Network Approach

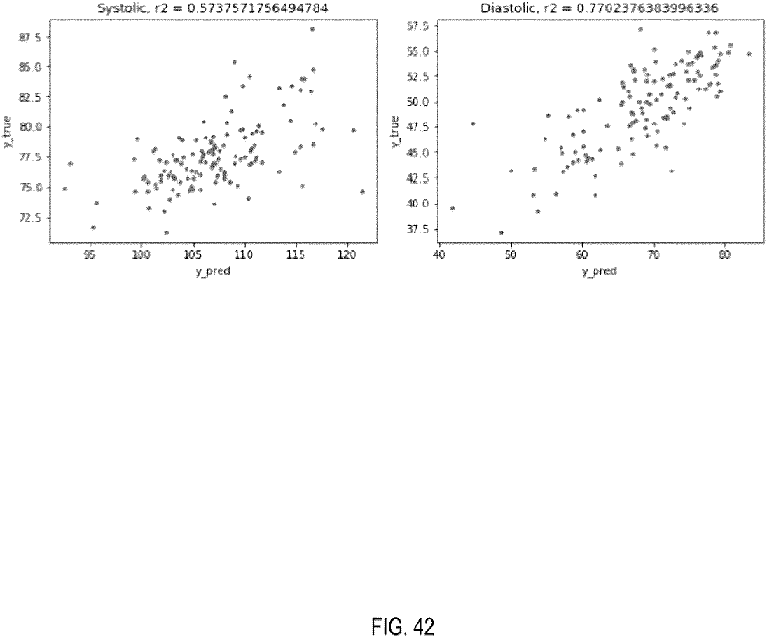

For this approach, features and labels were constructed as described in Section 7.4 The 1D CNN model used to calculate BP was very similar to the one used to classify lung volume, with 2 convolutional layers, a max pooling layer and dropout regularization to improve generalization. The main difference with this model was that a linear activation function with two channels (for prediction of systolic and diastolic BP) was used at the output instead of a softmax activation function (for prediction of HLV or LLV). The linear activation function is better suited in this case because the problem is now a regression problem instead of a classification problem. The model was trained with a mean absolute error loss function and an Adam optimizer. This model was evaluated on the test set of 125 cardiac cycles from one subject and achieved an r2 score of 0.55 for systolic BP and 0.67 for diastolic BP. The correlation plots are shown in FIG. 42.

FIG. 42: Correlation plots of the 1D CNN predictions for systolic and diastolic BP.

### 7.7 Conclusion

The best ML results obtained for BP calculation were from the 1D CNN approach. While the r2 scores are not exceptionally high, they are higher than expected considering that a relatively simple CNN architecture was used to statistically model a relatively complex biological process. Therefore, our next steps are as follows:
Increase the complexity of the CNN architecture being used, as this generally results in more accurate predictions.
Train the model for more time, as this will also increase the accuracy

Algorithms and techniques for identifying and detecting vibrations corresponding to cardiac phase transitions using VCG will now be described. Such algorithms and techniques may be implemented by the systems and methods for blood pressure measurement described herein, such as the systems and methods described in FIGS. 2-6. For example, the algorithms and techniques may be performed by the real-time signal processing unit 318 of the system 300 of FIG. 4 or the computer system 400 of FIG. 5 (e.g. vibrational pulse identifier module 424).

Non-Invasive Identification of Cardiac Phase Transitions using Vibrational Cardiography.

Cardiography is a necessary component of diagnostic and preventive care because it enables the measurement of cardiac time intervals which indicate the phases of the cardiac cycle. These phase transitions induce valve movements, which are manifested as vibrations at the sternum. Objective: Automatically identify the vibrations corresponding to cardiac phase transitions. Methods: Cardiac activity was monitored for subjects while at rest, during exertion, and while performing static breath holds. The subjects consisted of males and females with no known cardiorespiratory ailments. Cardiac activity was recorded via concurrent vibrational cardiography (VCG), electrocardiography (ECG), and impedance cardiography (ICG). The raw acceleration and gyration components of the VCG signal were processed into quantities representing their linear jerk and rotational acceleration, respectively. This mathematical transformation increased the signal to noise ratio of the vibrational pulses in the morphology of the VCG waveform. Results: The timing of first vibrational pulse, V1 was referenced with the ECG R peak using heart rate (HR) as a figure of merit. The timing of the second vibrational pulse, V2, was compared with the ICG X point. Its identification was evaluated using two figures of merit: its delay from the ECG R peak and the time interval between both pulses. Conclusion: The vibrational pulses that occur during cardiac phase transitions are automatically identifiable using VCG. Significance: This study demonstrates the feasibility of using VCG in analyzing mechanical cardiovascular function. It facilitates portable, non invasive cardiac monitoring in daily life.

Cardiovascular diseases are the largest contributor to mortality rates in developed countries. This is because the symptoms of a malfunctioning heart are often inconspicuous and remain undetected. As a result, cardiac issues are typically diagnosed at a later stage, which adversely affects the cost of treatment and the likelihood of success. Such treatment poses a significant burden on healthcare systems. The problem, however, is not necessarily the disease itself. Medical studies have shown that cardiovascular diseases are treatable at an early stage and certain complications can even be detected prior to their onset. Furthermore, economic studies have shown that the cost of treating the disease is drastically higher than preventing it. Prevention requires regular monitoring to enable the detection of early stage symptoms. Regular cardiac monitoring therefore has the potential to aid the diagnosis, analysis, and prevention of cardiac ailments. However, even with regular clinical check-ups, diagnostic accuracy is limited by situational, physiological, and interpersonal variability. This stresses a need for continuous, wearable monitoring. Autonomous cardiac monitors have been shown to enable the detection of irregular and anomalous activity, which could then inform prevention and treatment strategies.

The primary metric for monitoring cardiac activity is heart rate (HR), that is, the frequency of cardiac cycles per minute. The gold standard for HR measurement is Electrocardiography (ECG). ECG records the electrical activity of the heart including cardiac depolarization at the beginning of each cycle. This electrical impulse distinguishes individual cycles and thereby enables the measurement of HR. Within each cycle, however, the transition from the systolic to diastolic phase is undetectable via ECG. Since the durations of these phases are key indicators of left ventricular performance, this limits the utility of ECG in analyzing cardiac function. In situations where a more comprehensive analysis is required, mechanical cardiac activity is typically measured via Echocardiography (EcCG). However, the complexity, size, and cost of EcCG instrumentation limits its utility to trained technicians in dedicated laboratories. These limitations present an opportunity for complementary methods of non-invasive, accessible cardiac monitoring. An indirect approach to this problem is to interpret cardiac phase transitions from their associated valve operation or blood flow.

Transitions between the systolic and diastolic phases are induced by the cardiac blood pressure cycle. These pressure differentials induce the hydraulic opening and closing of cardiac valves. Valve operation regulates blood flow through the heart and therefore determines the phase of the cardiac cycle. The change in impedance caused by the volume of blood flowing through the heart can be detected via impedance cardiography (ICG). While ICG is capable measuring cardiac phase transitions, the sensing method requires 6 dual electrode placements and is susceptible to motion artifact. Alternatively, during a phase transition, the movement associated with valve operation generates mechanical compression waves. These waves diffuse through the chest and are manifested as vibrations at the surface of the skin. High vibrational amplitudes have been recorded at the xiphoid process of the sternum due to its proximity to the heart. These vibrations can be detected by commercially available inertial measurement units (IMUs) due to their ability to leverage recent developments in micro-electro-mechanical systems and motion sensing technology. This method of recording cardiac-induced sternal vibrations is termed Vibrational Cardiography (VCG). A VCG recording consists of three dimensional (3D) linear acceleration and 3D gyration, which are separately known as Seismocardiography (SCG) and Gyrocardiography (GCG), respectively. The potential of VCG lies in its capacity to monitor the vibrations caused by mechanical cardiovascular function in 6 degrees of freedom from a single IMU. Strong oscillatory features have consistently been observed in two segments of the VCG waveform pertaining to each cardiac cycle. The timings of fiducial points in these features have been found to coincide with cardiac valve events detected via EcCG. These coincidences suggest the possibility of using VCG for cardiac monitoring and specifically, to identify cardiac phase transitions.

Our work involved the development of an algorithm that increased the signal to noise ratio (SNR) of cardiac induced vibrations in VCG waveforms. It was used to identify the two prominent vibrational pulses, V1 and V2, which have been shown to coincide with cardiac phase transitions. In this context, the accuracy of our algorithm was evaluated using measurements obtained via ECG and ICG. The transition from the diastolic to the systolic phase was referenced with the ECG R peak whereas the transition from the systolic to the diastolic phase was referenced with the ICG X point. While the ICG B point could have been used to indicate transitions from the diastolic to the systolic phase, the low quality of the recordings rendered ICG measurements to be heavily dependent on its annotation scheme. This variability necessitated the use of ECG as a reliable reference. Despite the subjectivity of ICG measurements, its baseline accuracy made it suitable for comparative measurements. Our choice of these two reference methods was based on their feasibility and accuracy in a laboratory environment as well as their electrical nature, which rendered them less susceptible to the same sources of noise as mechanical VCG.

The objective of this study was to automatically identify both V1 and V2 in order to provide a basis for future analysis concerning the information contained in these vibrations. Previous VCG classification algorithms have focused on the vibration corresponding to V1. Their accuracy was evaluated against R peak detection from concurrent ECG by using the instantaneous HR as a figure of merit. Algorithms that have attempted the identification of V2 appear to have conducted this work as part of a larger analysis. As a result, these algorithms provide feature recognition concerning V2 that is either without a reference measurement or an automatic identification protocol. In this context, our work demonstrates the first algorithm designed to identify both the vibrational pulses associated with cardiac phase transitions in a VCG waveform. In adherence with standard practices, we evaluated the accuracy of our VCG V1 pulse identification protocol and ICG B point annotation scheme by referencing the timings with ECG R peaks. In an analogous manner, the identification of V2 pulses was then compared with valid annotations of the ICG X points. This study extends our preliminary work on vibrational pulse extraction by improving the accuracy of the algorithm and further analyzing the validity of VCG in identifying cardiac phase transitions. The algorithm was developed from a custom algorithm for V1 identification which was designed for an electromechanical cardiac monitor that recorded both VCG and ECG. Based on the accuracy of our algorithm for V1 detection as well as the similarities that were observed between V1 and V2, we extended the previously developed algorithm into a novel technique for the identification of both V1 and V2 pulses within the VCG waveform.

### II. Methods

### A. Experimental Setup and Procedure

All experiments were conducted with the approval of the Review Ethics Board at McGill University. The subjects were tested in a supine position. The sequence of tests included a 3-minute baseline recording at rest, two static breath holds at high and low lung volume (inhaled and exhaled), and a 5-minute recovery test. The recovery test was conducted immediately after the subject performed bicycle kicks until sufficiently exerted. Cardiac activity was recorded as VCG, ECG and ICG.

Cardiac-induced vibrational waves were detected on the surface of the skin by a six-axis motion sensor (MPU 9250, Invensense). The motion sensor was controlled by a Raspberry Pi microcontroller (Pi Zero W, Raspberry), and its sampling frequency ƒₛ was 550 Hz. The accelerometer and gyroscope sensitivities were set to ±2g and ±250°/s, respectively, in order to detect vibrations. The sensor was placed at the xiphoid process of the sternum with its Z-axis oriented outward along the dorsoventral axis of the body. Its exact position in reference to the heart was indeterminable during testing. The experimental setup shown in 43 was assembled to evaluate the cardiac activity of the subjects via concurrent VCG, ICG, and ECG.

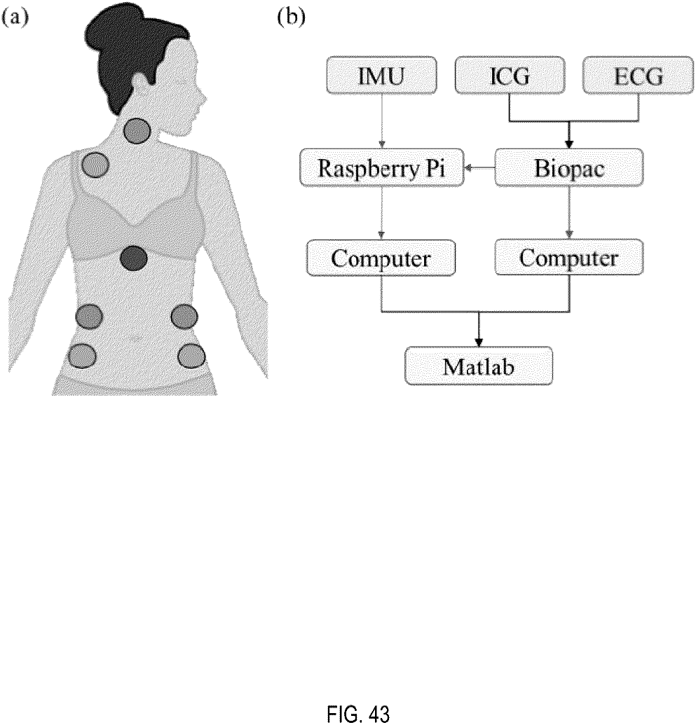

FIG. 43 illustrates (a) General placement of the ICG electrodes (green), ECG electrodes (blue), and VCG sensor (red), (b) System configuration enabling simultaneous recordings of ECG, ICG, and VCG.

Reference measurements were obtained from simultaneous ECG and ICG recordings using a multichannel Biopac analog-to-digital converter (ADC) (MP160WS, Biopac), which was used as the acquisition unit. The ECG and ICG electrodes were attached to the torso and neck in their standard positions. The signals were acquired by modules (ECG100C, Biopac, and NlCO100C, Biopac) which were wirelessly connected with the acquisition unit. VCG recordings were synchronized using a pulse generated by the unit. The raw data was filtered using a combination of software (AcqKnowledge 5, Biopac) and custom algorithms (R2019A, Matlab). The ECG signal and a filtered ICG signal were automatically annotated by the software. However, heavy overfitting in the ICG annotation resulted in numerous false positives that required further processing. The B- and X-points annotated in the ICG waveform were filtered based on their proximity to an R-peak in the ECG waveform of the same cardiac cycle.

### B. Transformation of the Vibrational Cardiogram

Transformation of the vibrational cardiogram, including one or more of the steps detailed below, may be used, for example, by the systems and methods described in FIGS. 2-6 to extract vibrational pulses corresponding to cardiac phase transitions from VCG data. For example, the signal processing unit 318 of the sensor interface computing device 314 of FIG. 4 may be configured to perform various vibrational cardiogram transformation steps and operations described herein via one or more software modules. Similarly, the computer system 400 of FIG. 5 may be configured to implement and perform various vibrational cardiogram transformation steps and operations via one or more software modules located at the processor 404. Extraction of vibrational pulses using vibrational cardiogram transformation may include extracting physical quantities representing linear jerk and rotational acceleration derived from the VCG signal. This may be performed for each cardiac cycle. The extracted physical quantities may comprise processed waveforms. The processed waveforms each include a pair of peaks which correspond with the expected occurrence of cardiac phase transitions. Accordingly, the system, such as computing device 314 or computer system 400, is configured to recognize such peaks as indicators of cardiac phase transitions and can use such information in the determination of a blood pressure measurement. In a particular embodiment, one or more of the systems and methods described in FIGS. 2-6 may implement the signal processing steps of FIG. 44, described below.

Processing the VCG signal may include selecting and processing only a subset of the linear acceleration component and a subset of the rotation component. For example, in an embodiment, this includes selecting and processing a single axis component of the linear acceleration component and two axes components of the gyration component. In a particular case, the single axis component of the linear acceleration component is a Z-axis component and the two axes components of the gyration component are an X-axis component and a Y-axis component. Jerk and rotational acceleration data may be determined for the selected linear acceleration and gyration components, respectively. For the jerk and rotational acceleration waveforms, peaks (e.g. Lorentzian) are identified therein which are centered at approximately the same timestamps. The identified peaks can be attributed to mechanical activity occurring during cardiac phase transitions.

Cardiac-induced longitudinal and shear infrasonic vibrations that propagated to the sternum were recorded as VCG. Respiratory effects were filtered out and lower frequencies in the range 0.6-20 Hz were attributed to ventricular contractions. Frequencies higher than 18 Hz were attributed to valve operation and consequently, the vibrational pulses associated with heart sounds. Since the spectral content of the mechanical oscillations was contained below 50 Hz, the VCG signal was down-sampled to 200 Hz using linear interpolation. This served two purposes. Suppressing spectral components beyond 100 Hz mitigated high frequency noise. Additionally, standardizing fₛ ensured a consistent acquisition rate and faster computational time.

The acquired signal consisted of 6 orthogonal degrees of freedom representing the linear and rotational components of three-dimensional (3D) motion. The linear component was measured as acceleration and the rotational component as gyration. The vectorial components representing cardiac-induced motion, a̅_{SCG} and g̅_{GCG}, could therefore be extracted as vectorial projections onto the coordinate axes of the sensor signal,

$$\begin{matrix}{\vec{a}}_{\text{SCG}} = {a}_{\text{X}} \hat{x} + {a}_{\text{Y}} \hat{y} + {a}_{\text{Z}} \hat{z} \\ {\vec{g}}_{\text{GCG}} = {g}_{\text{X}} {\hat{θ}}_{\text{X}} + {g}_{\text{Y}} {\hat{θ}}_{\text{Y}} + {a}_{\text{Z}} {\hat{θ}}_{\text{Z}}\end{matrix} \qquad \text{(1)}$$

The linear and angular components of the VCG signal required separate processing techniques to retain their fidelity since they were recorded in different units of motion.

### 1. Processing the SCG Signal

Based on the sensor orientation, the Z-axis of the accelerometer aligned with the dorsoventral axis of the body. Hence, the SCG acceleration vector a_{SCG} was assumed to be projected mainly onto the Z-axis. To reflect this, the magnitude of the overall a_{SCG} component was retrieved from its projections onto the coordinate axes as,

$${\vec{a}}_{\text{SCG}} = \frac{{a}_{\text{Z}}}{\left|{a}_{\text{Z}}\right|} \sqrt{{a}_{\text{X}}^{2} + {a}_{\text{Y}}^{2} + {a}_{\text{Z}}^{2}} \qquad \text{(2)}$$

The direction of the a_{Z} component was retained in order to preserve the occurrence of fiducial features associated with cardiac-induced vibrations. The purpose of this step was to filter out motion artifacts and sensor noise that were present in any individual axis. Despite retaining the a_{Z} component, individual cardiac events were indistinguishable from the oscillatory waveforms due to a high inter-subject variability. This decreased the value of applying a standardized feature recognition algorithm. Hence, the waveform morphology was instead processed to identify vibrational pulses.

The human body was assumed to consist of elastically deformable matter in a quasi-static state. In such a material, a changing acceleration would directly indicate the presence of mechanical waves. Hence, cardiac-induced vibrational pulses could be identified by the rate of change of acceleration, or jerk. To extract an effective jerk waveform, the a_{SCG} waveform was differentiated and maximized within a certain time window by using the equation,

$$\begin{matrix}{j}_{\text{SCG}} \left(t\right) = max \left(\frac{{a}_{S C G} \left(t\right) - {a}_{\text{SCG}} \left({t}^{′}\right)}{t - {t}^{′}}\right) \\ \text{where} \left(t - {t}^{′}\right) ∈ \left[0.02 s , 0.05 s\right]\end{matrix} \qquad \text{(3)}$$

A comparatively high oscillation amplitude was classified as a cardiac-induced vibrational pulse if its frequency was within the experimentally verified range between 10-50 Hz. In the context of the algorithm, each data point was evaluated based on its relationship with other points in the signal within a window of 0.02-0.05 s from itself. Any slow-varying oscillations were automatically suppressed by the correspondingly larger time period between timestamps in the denominator of the equation. The GCG signal was processed in a similar manner.

### 2. Processing the GCG Signal

Based on the sensor orientation and the orthogonality between g̅_{GCG} and a̅_{SCG} motion, the GCG vector was projected mainly onto the complementary X and Y gyration axes with a negligible component in the Z axis. This is why the Z component was neglected. The GCG signal was consequently retrieved as,

$${\vec{g}}_{\text{GCG}} = {g}_{X} {\hat{θ}}_{X} + {g}_{Y} {\hat{θ}}_{Y} \qquad \text{(4)}$$

Each axis was processed separately to convert it to acceleration. The spectral window for gyration was slightly different.

$$\begin{matrix}{a}_{\text{GCG}} \left(t\right) = max \left(\frac{{g}_{\text{GCG}} \left(t\right) - {g}_{\text{GCG}} \left({t}^{′}\right)}{t - {t}^{′}}\right) \\ \text{where} \left(\text{t} - {\text{t}}^{′}\right) ∈ \left[? s , ? s\right]\end{matrix} \qquad \text{(5)}$$

These mathematical transformations optimized the signal-to-noise ratio (SNR) of the oscillatory features in the signals. In this manner, the vibrational pulses V₁ and V₂ were extracted as impulses from VCG waveforms.

### 3. Identification of Vibrational Pulses

Individual cardiac cycles were separated by their corresponding R-peaks in the ECG waveform. An offset of 0.05 s was added prior to each R-peak to account for the beginning of the pulse. In each cardiac cycle, physical quantities representing linear jerk, j_{SCG}, and rotational acceleration, α_{GCG},_{X} and α_{GCG,Y}, were derived from the VCG signal. Each of the three processed waveforms produced a pair of peaks that coincided with the expected occurrence of cardiac phase transitions. The Lorentzian shape of the peaks confirmed an increased SNR. Each waveform was therefore fitted with a series of two Lorentzian functions within each cardiac cycle as defined by,

$$L \left(t\right) = {\sum }_{i = 1}^{2} {p}_{3 , i} \frac{{\left({p}_{2 , i} / 2\right)}^{2}}{{\left(t - {p}_{1 , i}\right)}^{2} + {\left({p}_{2 , i} / 2\right)}^{2}} \qquad \text{(6)}$$

The coefficients p₁, p₂ and p₃ represent the center, full width at half maximum, and height, respectively of each Lorentzian function. The subscript i simply denotes the order of the vibrational pulse as V₁ or V₂.

Together, these equations were implemented in the signal processing steps shown in 44.

FIG. 44 illustrates signal processing steps used to obtain the vibrational pulses V₁ and V₂ from the acquired vibrational motion signal represented as VCG, according to an embodiment.

The first Lorentzian, V1, was expected to occur within 0.1 s of an ECG R peak. Hence, its position was evaluated using ECG as a reference. The time period between successive V₁ pulses was interpreted as the beat-to-beat duration (BTB), which was converted to an HR measurement. Similarly, V₂ was expected to occur around the middle of the cycle based on the fraction of BTB occupied by LVET, that is, the LVET fraction (LVETF). The time period between V₁ and V₂ in each cardiac cycle was interpreted as LVET. The position of each pulse was further cross-verified between SCG and GCG.

### III. Results

The experimental data consisted of 58 tests (2 recovery sets were discarded due to acquisition errors) conducted for 15 subjects over a total of 7892.228 s.

### A. Signal Filtration

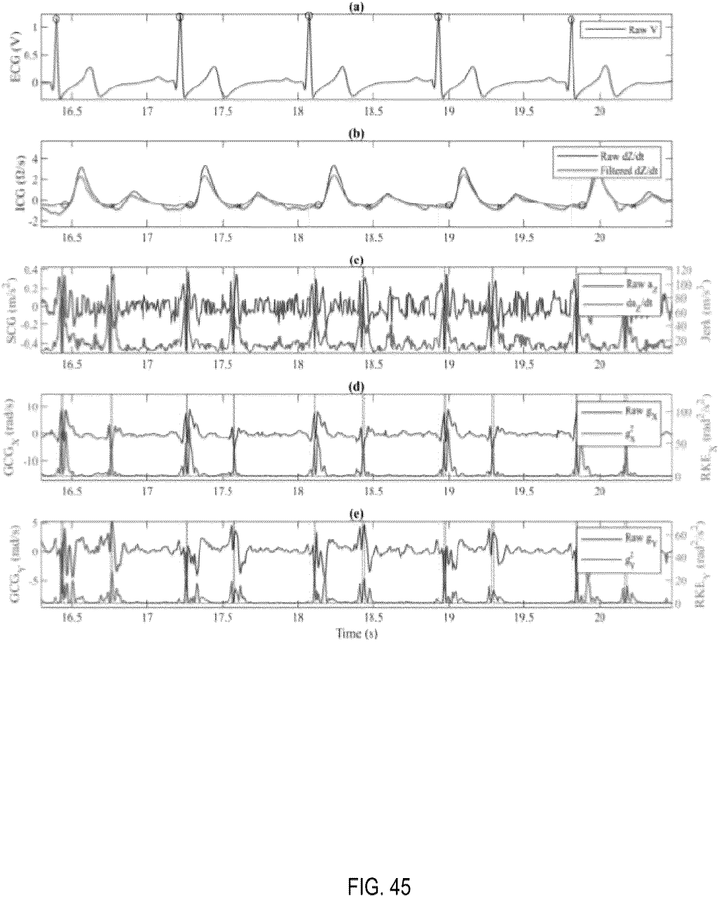

Our goal was to derive a physically valid mathematical transformation that improved the signal to noise ratio (SNR) of V₁ and V₂ in the a_{Z}, g_{X} and g_{Y} components. In this context, the filter was designed to transform the raw signals (blue) in FIG. 45(c)-(e) into impulses which retained their vibrational content. These impulses were identified by their Lorentzian distribution in the processed signals (red), which represented the linear jerk and rotational kinetic energy (RKE) of the vibrations.

FIG. 45 illustrates simultaneous recordings of (a) ECG with circles representing the identified R-peaks; (b) Raw (blue) and filtered (red) ICG with the annotated B- and X-points shown as circles and crosses respectively; and (c) SCG acceleration a_{Z} (blue) and jerk magnitude |da_{Z}/dt| (red), (d) X-axis GCG gₓ (blue) and its RKE component

$${g}_{\text{X}}^{2}$$

(red), and (e) g_{Y} (blue) and

$${g}_{\text{Y}}^{2}$$

(red) with dotted, black lines representing the identified timestamps of V₁ and V₂.

### B. Instantaneous Heart Rate From V₁

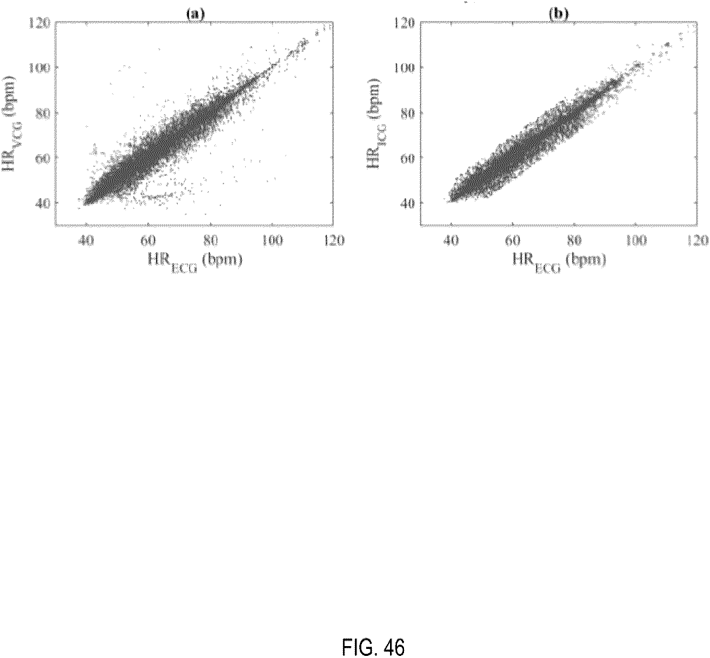

Cardiac activity was recorded using ECG, ICG, and VCG. Each recording was then analyzed separately. For the purpose of this study, the processed ICG and VCG signals were annotated in reference to the R-peaks in the ECG signal. Instantaneous HR was calculated from the time interval between R-peaks for ECG, B-points for ICG, and V₁-peaks for VCG. Their correlations are shown in FIG. 46.

FIG. 46 illustrates correlation of HR calculated from (a) VCG and (b) ICG with a r² of 0.9887 and 0.9824 respectively, when referenced with ECG.

Outliers at the end of each recording were discarded as a result of acquisition inconsistencies. Additionally, HR_{ICG} measurements that differed from HR_{ECG} by 10 bpm were excluded from further evaluation. HR_{VCG} was found to exhibit a larger spread. Despite this, the accuracy of HR_{VCG} produced a r² of 0.9887 versus 0.9824 for HR_{ICG} when referenced with HR_{ECG}. This was attributed to the fact that the exact location of the B-point in the ICG signal was not as evident as the V₁ peaks identified in VCG or the R-peaks from ECG.

### C. Left Ventricular Ejection Time From V₂

The vibrational pulses detected as V₁ and V₂ were expected to indicate cardiac phase transitions, which implied that the time period between them reflected the LVET. However, due to the inconsistency of ICG annotation, a correlation coefficient using LVET measurements did not directly provide any insight. Instead, the accuracy of V₂ detection was compared using two metrics: (i) the distance of the X and V₂ points from their corresponding R-peak demonstrated the ability of each technique to identify the end of the systolic phase, and (ii) the LVETF indicated the ability of B-X and V₁-V₂ to reflect BTB. Using these two figures of merit, the vibrational pulses derived from VCG were compared with ICG as shown in FIG. 7.

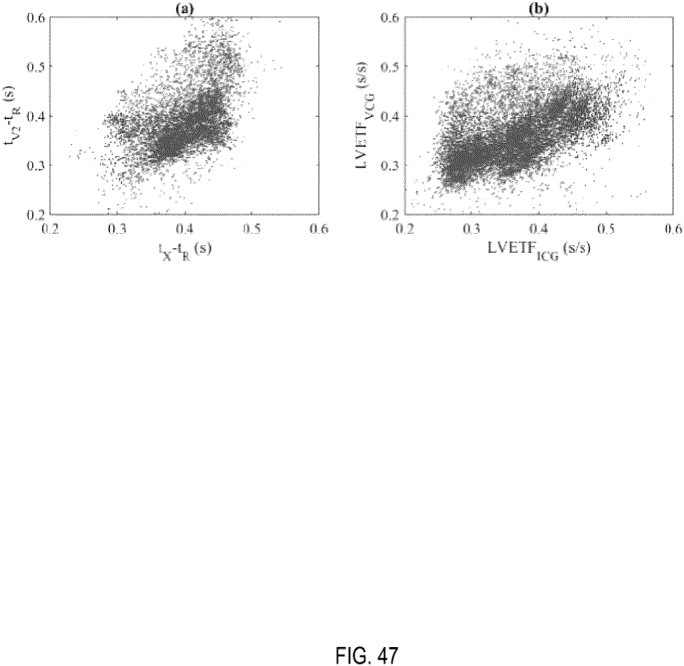

FIG. 47 illustrates correlation of (a) the time interval from the ECG R-peak to both V₂ from VCG and B from ICG, and (b) LVETF obtained from VCG and ICG with a r² of 0.251 and 0.2797 respectively.

The identification of V₂ from VCG waveforms produced comparable results with concurrent ICG measurements. The agreement between HR measurements for ICG and VCG suggested the potential of incorporating an initial calibration period to improve identification accuracy. This would reduce outliers in the ICG annotation generated by the software, thereby providing a more accurate reference.

### IV. Discussion

Cardiac-induced vibrations propagating through the chest were detected by an inertial measurement unit consisting of an accelerometer and a gyroscope. The signal also contained noise from motion artifact and respiration due to the high sensitivity setting of the sensor. To mitigate this effect, the raw signals were converted to physical quantities that directly increased the SNR of the vibrational pulses.

The SCG signal was differentiated to derive the jerk magnitude from its slope. This quantity was derived from the force contained in the vibrations. The height and width of the pulse reflected its strength and jump-discontinuity, respectively. Instead of conventional spectral filtering, a frequency filter was directly incorporated into the jerk calculation. A similar process was used to calculate the rotational acceleration of the two GCG axes. All three processed waveforms exhibited Lorentzian peaks which were centered at approximately the same timestamps. The occurrence of this triplet of coinciding peaks indicated the possibility of a common mechanical origin between them. This origin was attributed to the mechanical activity occurring during cardiac phase transitions. The prominence of these Lorentzian peaks confirmed the validity of this approach.

These vibrational pulses were known to be caused by cardiac mechanical activity. The activity was speculated to be a combination of valve operation and the pressure exerted by the ventricles on the aorta and chest wall. This motion represented the kinetic energy generated by the ventricle for each stroke volume of blood. Hence, the linear kinetic energy (KE) was also considered as a possible candidate for SNR amplification. Assuming the mass of the sensor, torso, and stroke volume of blood to be constant during each cardiac cycle, the linear of the vibrational pulse would be reflected in the integral of the acceleration waveform, that is, its linear velocity. However, the accuracy of the linear velocity calculation was physically limited by its dependence on the initial velocity, which was unknown. Hence, a waveform reflecting linear KE could not be derived because the result was prone to drift with sensor noise and bias. Alternatively, the a_{Z} component of cardiac vibrations was known to exhibit characteristic oscillations coinciding with the occurrence of heart sounds. This implied that vibrational pulses could be identified by the rate of change of acceleration, or jerk. The rotational kinetic energy of the vibrational waveform was also considered as a possible candidate for SNR amplification since up to 60% of the vibrational energy was contained in the rotational component of the signal. Assuming the moment of inertia to be constant during each cycle, the rotational kinetic energy of the vibrational pulse would be reflected in the gyrational g̅_{GCG} waveform. Hence, in our previous work, the gₓ and g_{Y} components were squared to obtain their energy profile. However, due to the mathematical nature of the square function, zero crossings in the signal were also zeroed in the rotational kinetic energy waveform. This caused the waveform to split into two peaks for every oscillation. Such behaviour did not serve the purpose of the algorithm which was to convert each vibrational pulse into a single, identifiable delta function. In this context, the conversion to rotational kinetic energy did not appropriately emphasize the oscillatory features in the GCG signal. This is why the gyration signal was also differentiated, which produced a waveform reflecting rotational acceleration.

The timings of the peaks associated with primary heart sounds were compared with concurrent ECG and ICG recordings. For the second heart sound, LVETF was used as a figure of merit to account for the different physical origin of VCG and ICG signals. Additionally, their ability to identify the end of the systolic phase was evaluated in reference to the ECG R-peaks. The most notable discrepancy between the measurements obtained from ICG and VCG was found to be of physiological origin. Slow-varying oscillations of the BTB measurement in response to respiration would typically be reflected in LVET assuming a relatively constant LVETF. Yet while LVET_{ICG} was modulated by this effect, LVET_{VCG} was not. However, the heights of the Lorentzian peaks in the VCG signal exhibited slowly varying oscillations corresponding to respiration volume. This selective effect of respiration on VCG morphology was attributed to an outward pressure on the heart exerted by lung expansion, which was assumed to proportionately amplify the force exerted on the chest wall as the heart compressed against it. The changing densities of the organic materials along the path of vibrational wave propagation could therefore have increased the pulse amplitude while modifying its group velocity.

Our findings suggest that by using a combination of VCG and ECG in an electro-mechanical cardiac monitor, a deeper analysis of cardiac activity could be conducted. This is because of the content-rich information obtained from coupled electrical ECG and mechanical VCG signals, as well as the high accuracy of both measurement methods. Since these vibrational waves apparently retain much of their characteristic signal profile after propagation, such an analysis could provide new insights into the vibrational energy imparted by cardiac activity.

### V. Conclusion

Heart monitors are a vital tool in the maintenance and improvement of cardiac health. The primary metrics for evaluating cardiac function are HR and LVET. We investigated the feasibility of using VCG in tracking these metrics. The SNR of the vibrational pulses V₁ and V₂ was improved using a novel algorithm. The experimental data consisted of 5129 cardiac cycles with an average BTB of 0.99 s corresponding to a HR of 60.49 bpm. The identification accuracy of V₁ produced squared correlation coefficients of 0.9824 and 0.9887 for instantaneous HR measured from ICG and VCG waveforms respectively, in reference with concurrent ECG measurements. The correlation for V₂ was 0.251 when comparing VCG and ICG to ECG using the (t_{V₂} - t_{R}) and (t_{X} - t_{R}) time periods respectively, and 0.2797 when comparing LVETF measurements. These results demonstrate the potential of VCG in detecting and analyzing the mechanical activity of the heart. Additionally, the form factor and cost of VCG motion sensors makes them an attractive option for applications in wearable sensing and autonomous healthcare.

Algorithms and techniques for neural network-based classification of static lung volume states using VCG will now be described. Such algorithms and techniques may be implemented by the systems and methods for blood pressure measurement described herein, such as the systems and methods described in FIGS. 2-6. For example, the algorithms and techniques may be performed by the sensor interface device 314 (e.g. via the real-time signal processing unit) or the data analytics server 328 of FIG. 4 or the computer system 400 of FIG. 5.

Non-invasive health monitoring has the potential to improve the delivery and efficiency of medical treatment. Objective: This study was aimed at developing a neural network to classify the lung volume state of a subject (i.e. high lung volume (HLV) or low lung volume (LLV), where the subject had fully inhaled or exhaled, respectively) by analyzing cardiac cycles extracted from vibrational cardiography (VCG) signals. Methods: A total of 15619 cardiac cycles were recorded from 50 subjects, of which 9989 cycles were recorded in the HLV state and the remaining 5630 cycles were recorded in the LLV state.A1D convolutional neural network (CNN) was employed to classify the lung volume state of these cardiac cycles. Results: The CNN model was evaluated using a train/test split of 80/20 on the data. The developed model was able to correctly classify the lung volume state of 99.4% of the testing data. Conclusion: VCG cardiac cycles can be classified based on lung volume state using a CNN. Significance: These results provide evidence of a correlation between VCG and respiration volume, which could inform further analysis into VCG-based cardio-respiratory monitoring.

### I. Introduction

Cardiovascular disease (CVD) is the leading contributor to global mortality rates. The pervasiveness of CVD-related incidents, coupled with the fact that preventive care has the potential to reduce mortality rates by millions and economic losses by trillions, has incited the medical community to seek preventative measures in tackling CVD. Non-invasive, continuous health monitoring could hasten diagnoses, improve preventative care and save lives by leveraging algorithms that connect physiological signals to cardiovascular health state trajectories. The potential of machine learning (ML) algorithms to classify such trends is evident. However, despite a wide variety of available techniques for cardiac and respiratory monitoring, the task is still nontrivial for nonmedical scenarios. This demonstrates the need for a noninvasive, continuous method of cardio-respiratory monitoring that will not significantly interfere with daily life while remaining accurate. In this study, we investigate vibrational cardiography (VCG) as a possible solution.

Cardio-respiratory activity generates thoracic vibrations that propagate through the chest wall. These vibrations can be recorded non-invasively by an accelerometer attached to the skin at the xiphoid process of the sternum, where vibrational signals are strongest due to the position of the heart in the thorax. The recorded accelerometer signal is called a seismocardiography (SCG) signal. Recently, microelectro- mechanical systems (MEMS) based motion tracking technology has improved to a point where both an accelerometer and a gyroscope have been integrated into a single miniaturized inertial measurement unit (IMU). This provided an integrated, coupled gyration signal, which motivated research in gyrocardiography (GCG) as a complementary measurement.

It has been shown that over 50% of the total kinetic energy transferred from the heart to the body is contained in the GCG signal, which shows the added value of GCG in monitoring cardiac activity. Additionally, since the two measurements are mutually orthogonal, there is an inherent difference in the noise characteristics to which each measurement is vulnerable. This facilitates a more comprehensive analysis when combining information from both. Therefore, in this work we have utilized vibrational cardiography (VCG).

Multiple studies use electrocardiography (ECG) or SCG to extract respiratory information such as respiration rate or respiration phase, but none of them have done so on a beat-to-beat basis using VCG. The closest works to this study were, in which the effect of static respiration volume on VCG signal morphology was investigated and, in which a machine learning approach was employed on SCG to identify respiratory phase on a beat-to-beat basis.

This paper presents a novel method for classifying high lung volume (HLV) versus low lung volume (LLV) on a beatto- beat basis by analysing the corresponding VCG cardiac cycles (CC). Our approach is based on convolutional neural networks (CNN). To classify the corresponding lung volume state of each CC, a 1D CNN was used. CNNs make use of convolving filters that are applied to local features. A certain degree of shift, scale and distortion invariance is ensured by forcing the extraction of local features. Although they were originally proposed for computer vision tasks, CNN models and architectures have since been proven to be significantly effective in many other applications. A 1D CNN uses 1D filters instead of 2D filters. These models are especially useful for analyzing data along the temporal dimension, hence why they were chosen for the task of analyzing VCG signals.

### II. Methodology

The morphology of SCG and VCG signals is dependent on respiration phase (i.e. inspiration versus expiration) and lung volume state (i.e. HLV versus LLV). Additionally, certain features of the SCG signal, such as amplitude and timing, change based on respiratory activity. These respiratory effects on SCG and VCG cause morphological dissimilarities with the potential to mask other signal variabilities that may be diagnostically valuable. Therefore, to reduce these dissimilarities, it is useful to group VCG signals based on lung volume, as each group would have similar waveform morphology. This could result in more accurate signal analysis and has the potential to increase the diagnostic value of VCG.

Here we define two distinct lung volume states; HLV, where the subject has fully inhaled and LLV, where the subject has fully exhaled.

### A. Data Collection

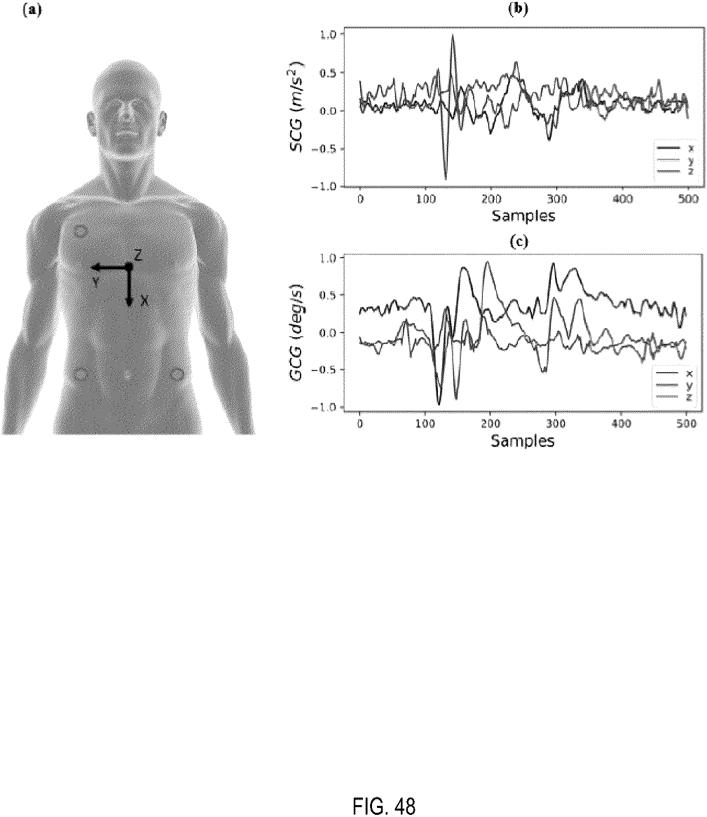

Experimental data were collected at McGill University with approval from the McGill Review Ethics Board. Inertial measurements were recorded by a 6 axis IMU (MPU 9250, InvenSense) attached to the xiphoid process of the sternum with a single piece of double-sided tape. The IMU was connected to a Raspberry Pi (Pi Zero W, Raspberry) to manage data collection. The positive X, Y and Z-axes of the accelerometer were oriented downward, right and outward, respectively. Consequently, the gyroscope coordinates followed the right-hand rule for rotation about these axes. Additionally, a BIOPAC system was used to concurrently record ECG. The ECG electrodes were attached to the skin in an Einthoven triangle on the torso. The described placement and orientation of the IMU and ECG electrodes is shown in FIG. 48(a). The corresponding signal morphology from this placement is shown in FIG. 48(b) for acceleration in all axis components and in FIG. 48(c) for gyration in all axis components.

Subjects were asked to hold their breath for as long as possible at both HLV and LLV, with a maximum of 2 minutes for HLV and 1 minute for LLV. The HLV holds involved inhaling as much as possible before holding, while the LLV holds involved exhaling as much as possible before holding. These holds were repeated twice more with rests in between, giving three HLV holds and three LLV holds per subject. All tests were performed with the subject in the supine position. The sample size was 50 participants and the average metrics of the study population can be seen in Table 5.

A recent study showed that morphological differences between SCG events was more dependent on lung volume state than respiratory flow (i.e. inspiration versus expiration). Therefore, our approach did not consider respiratory flow, with data collected from static breath holds as opposed to regular breathing.

FIG. 48: (a) Placement of the inertial measurement unit (IMU) on the xiphoid process of the sternum (shown in black) with its orientation represented by the Cartesian reference axis, and the electrocardiography (ECG) electrodes (shown in green) attached to the torso. The corresponding signal morphology of a single CC is shown for (b) acceleration in all axis components and (c) gyration in all axis components.

TABLE 5

Study Population

| | |
|---|---|
| Description | Value |
| Participants | 50 |
| Percent Male | 56% |
| Age | 24.4 ± 4.45 years |
| Weight | 69.1 ± 13.0 kg |
| Height | 172.6 ± 10.6 cm |

### B. Preprocessing

Preprocessing involved separating VCG signals into CCs and interpolating them to a uniform length of 500 samples per CC. The beginning of each CC was set as 0.02 seconds prior to the timestamp of the concurrently recorded ECG R-peak. This was done to approximately account for the onset of the P wave and consequently, the vibrations corresponding to the given CC. Matlab (R2019b) and Python packages Scikit-Learn and NumPy were used to preprocess the signals.

### C. Feature Construction

The uniform-length CC vectors were concatenated to form a preliminary n × m feature matrix for each axis component, where n was the number of cardiac cycles in the dataset and m was the number of elements per cardiac cycle (500 in this case). The preliminary feature matrix is shown in equation (1), where xₙ[m] represents the m^{th} element of the n^{th} CC.

$$\left[\begin{matrix}{x}_{⊥} \left[1\right] & {x}_{⊥} \left[2\right] & ⋯ & {x}_{⊥} \left[m\right] \\ ⋮ & ⋮ & ⋱ & ⋮ \\ {x}_{n} \left[1\right] & {x}_{n} \left[2\right] & ⋯ & {x}_{n} \left[m\right]\end{matrix}\right] \qquad \text{(1)}$$

This process was repeated for all six axis components, and the resulting feature arrays were concatenated along a third axis to form the final n × m × 6 feature matrix used for training.

The labels used for training were binary; with a 1 attributed to HLV cardiac cycles and a 0 attributed to LLV cardiac cycles.

### D. Convolutional Neural Networks

The developed CNN consisted of 2 convolutional layers, a max-pooling layer, a fully connected hidden layer and a fully connected output layer. Dropout regularization was used to improve generalization and reduce overfitting. The rectified linear unit (ReLU) was used as the activation function for the 2 convolutional layers and the first fully connected layer, and a softmax activation function was used at the output for classification.

The model was trained for 50 epochs with a sparse categorical cross-entropy function used as the loss function. The Adam optimizer was used to update network weights and dynamically change the learning rate hyperparameter. The overall architecture of the developed CNN is shown in

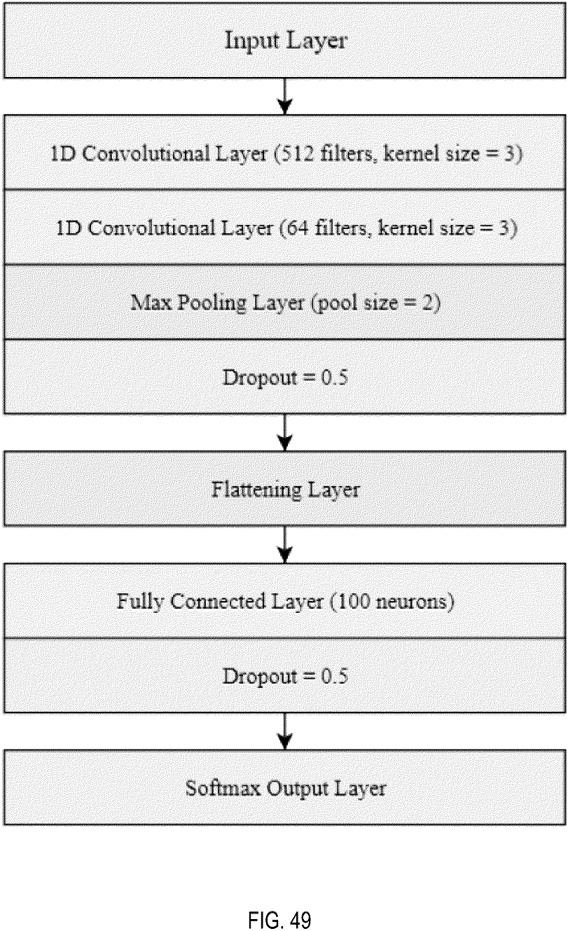

FIG. 49. Implementation of the CNN was performed by the Python package Keras.

FIG. 49: The overall architecture of our proposed CNN to classify lung volume state of VCG cardiac cycles.

### III. Results

In order to investigate the performance of our proposed CNN, the full dataset of 15619 CCs was randomly split into a training and testing set, with 12495 samples used to train and 3124 used to test (i.e. an 80/20 split).

The developed model was evaluated on the test set. As mentioned earlier, HLV was defined as 1 and LLV as 0 during feature construction. Therefore, an HLV cardiac cycle correctly predicted as HLV was labeled a true positive (TP), and an LLV cardiac cycle correctly predicted as LLV was labeled a true negative (TN). Additionally, an HLV cardiac cycle incorrectly predicted as LLV was labeled as a false negative (FN), and an LLV cardiac cycle incorrectly predicted as HLV was labeled as a false positive (FP).

Accuracy, precision and recall were evaluated to be 99.4%, 99.4% and 99.5% respectively, according to equations (2), (3) and (4). The resulting confusion matrix is shown in Table 6.

$$A c c u r a c y = \frac{\sum T P + \sum T N}{\sum T o t a l P o p u l a t i o n} \qquad \text{(2)}$$

$$P r e c i s i o n = \frac{\sum T P}{\sum T P + \sum F P} \qquad \text{(3)}$$

$$R e c a l l = \frac{\sum T P}{\sum T P + \sum F N} \qquad \text{(4)}$$

TABLE 6

Confusion matrix for classification results

| | Predicted LLV | Predicted HLV |
|---|---|---|
| Actual LLV | 1162 | 11 |
| Actual HLV | 9 | 1942 |

### IV. Discussion

Due to the high accuracy of the developed model in classifying lung volume state, our results reiterate the conclusions reached by, in which it was shown that differences in lung volume state (i.e. HLV versus LLV) result in a quantifiable distinction between VCG waveforms. The effects of lung volume state on VCG are hypothesized to include three interrelated mechanisms. First, the volume of air in the lungs may change how the vibrational waves from cardiac activity are modulated, thus affecting the morphology of the VCG signal recorded at the surface of the skin. Moreover, changes in lung volume cause intra-thoracic pressure changes, which may lead to variations in cardiac output. Finally, movement of the heart, lungs and diaphragm due to variations in lung volume state presumably change the position of the heart with respect to the IMU. While the exact contribution of each mechanism has not been determined, these and other mechanisms can cause complex changes in VCG morphology.

Our results are also an improvement on those obtained by, in which it was shown that a support vector machine (SVM) could classify lung volume state (i.e. HLV or LLV) from an SCG signal with 75% accuracy. However, it should be noted that this study utilized data from dynamic breathing as opposed to static breath holds. Our improved classification accuracy leads to three possible implications: 1) the added GCG in our analysis increased accuracy, 2) a CNN approach is more accurate for VCG analysis than an SVM approach and 3) classifying lung volume state using dynamic breath data is much less accurate than using static breath holds. The degree to which any of these implications affects classification accuracy is not clear. Therefore, this is a potential avenue for further investigation into VCG as it relates to lung volume state classification.

Additionally, this study has certain limitations. Firstly, data were acquired from subjects holding their breath as opposed to breathing normally. Therefore, it remains to be shown whether the proposed CNN could be adapted to perform similarly on data from dynamic breathing instead of static breath holds. Secondly, no cross-validation procedures were utilised as a model evaluation technique due to the long training times of the proposed CNN model. Finally, all data were collected with subjects laying in the supine position.

Therefore, it is not clear whether the CNN model would perform similarly on data from subjects in motion or in other positions.

### V. Conclusion

In this work, a 1D CNN architecture was shown as a methodology to classify VCG cardiac cycles based on lung volume state. VCG is a low-cost solution to non-invasive, continuous health monitoring. However, one of its limitations is that biological processes which cause lung volume state to affect VCG signal morphology are not completely understood. Therefore, it is useful to classify VCG cardiac cycles based on their corresponding lung volume state in order to reduce the morphological dissimilarity introduced by the volume of air in the lungs. The proposed CNN managed to learn the effect of these biological processes, without any understanding about how the effects arise. It enabled classification of the lung volume state of a given VCG cardiac cycle with an accuracy of 99.4%, which proved that lung volume created an identifiable variance from the CNN’s perspective.

The results of this work establish a basis for further investigation into VCG-derived lung volume information for continuous and non-invasive health monitoring. Such a health monitoring technique could increase the delivery, speed, access and efficiency of medical diagnoses and treatments.

Algorithms and techniques for heart rate estimation from VCG with different orientations will now be described. Such algorithms and techniques may be implemented by the systems and methods for blood pressure measurement described herein, such as the systems and methods described in FIGS. 2-6. For example, the algorithms and techniques may be performed by the real-time signal processing unit 318 of the system 300 of FIG. 4 or the computer system of FIG. 5.

Remote health monitoring is a widely discussed topic due to its potential to improve quality and delivery of medical treatment and the global increase in cardiovascular diseases. Objective: Seismocardiography and Gyrocardiography have been shown to provide reliable heart rate information. A simple and efficient setup was developed for the monitoring of mechanical signals at the sternum. An algorithm based in autocorrelation was run on subjects with different orientations in order to detect heart rate. Methods: Subjects performed several tests where both SCG and GCG were recorded using an inertial measurement unit, a Raspberry Pi and a BIOPAC acquisition system. A total of 2335 cardiac cycles were obtained from 5 subjects. Heart rate was determined on a per second basis and compared with an electrocardiography (ECG) reference by correlation coefficients. Ensemble averages were used to visualize differences in VCG morphology. Results: Heart rate estimation obtained from VCG signals across all 5 subjects was referenced with ECG and achieved an r-squared correlation coefficient of 0.956 when supine and 0.975 when standing, compared to 0.965 across the entire dataset. Conclusion: Autocorrelated Differential Algorithm was able to successfully detect heart rate, regardless of orientation and posture. Significance: Changes in orientation of the body during measurement introduce inaccuracies. This work shows that the algorithm is resistant to orientation and more adaptable to everyday life.

### I. Introduction

Continuous, remote health monitoring is prominent due to the global increase in cardiovascular disease as a leading cause of death. Complications with the heart could remain undetected for years before the impending ailment. Continuous monitoring over longer times potentially leads to early detection of irregularities in vital signs. This would provide the ability to predict ailments before they occur and offer a better chance at prevention. Moreover, momentary monitoring and diagnosis by health professionals is subject to inaccuracies due to changing cardiac activities depending on psychological or situational influences. Continuous monitoring would provide health professionals with more reliable information for more accurate diagnosis as well as measurement of vital signs during daily life activities (during work, at home, during sport activities, etc.).

These reasons have paved the way for researchers around the world to pursue non-invasive, continuous monitoring of the heart. Cardiac activity produces mechanical waves that propagate through the chest and can be measured at the skin using triaxial accelerometers and gyroscopes. These measurements are referred to as seismocardiography (SCG) and gyrocardiography (GCG) and have been proven to provide reliable information regarding cardiac mechanics, heart rate and sounds, while providing additional features such as Pre-Ejection Period and Left Ventricular Ejection Time. Micro-electro-mechanical systems solutions for motion tracking have offered the possibility of having both an accelerometer and a gyroscope in a single inertial measurement unit (IMU). This facilitates simultaneous measurement of both SCG and GCG, collectively termed Vibrational Cardiography (VCG).

The standard for detecting heart rate is electrocardiography (ECG) where the electric potentials at the heart are measured. However, ECG does not provide direct information as to the actual motion of the heart muscles. ECG provides a measurement of the surface electric potentials from which cardiac mechanics are assumed but not obtained. Alternatively, VCG records the mechanical motion of the heart through vibrations by integrating the six mutually orthogonal axes from both SCG and GCG in a more comprehensive vibrational signal. A combined SCG and GCG measurement was found to improve accuracy due to different noise rejection criteria in the signals.

A prominent problem with VCG signal morphology is that it tends to vary significantly due to several factors including age, gender, BMI, respiration and motion. Posture can distort the SCG signal due to changes in the mechanical vibration response of the body. These variables introduce inconsistencies. A deeper understanding would potentially lead to reducing VCG noise and providing more reliable data. The purpose of this paper is to analyze the effects that orientation has on heart rate detection when subjects are not constrained to the supine position. This paper acts as a pilot study to explore the feasibility of beat to beat estimation and classification of a subject’s orientation and posture. This work demonstrates the next step towards using VCG signals as an everyday cardiac monitoring technique as daily use requires recordings in more positions than just supine.

### II. Methods

### A. System Configuration

Cardiac-induced vibrations were detected by an IMU placed at the xiphoid process of the sternum. The IMU sensor is a nine-axis InvenSense Motion Processing Unit™ 9250. Only the triaxial gyroscope and accelerometer were used for this study. The Raspberry PI (RPI) Zero W was used to control the system. This RPI model employed a PIZ Uptime battery shield for power and wireless mobility to the user. The battery shield used a Li-Ion Rechargeable Battery.

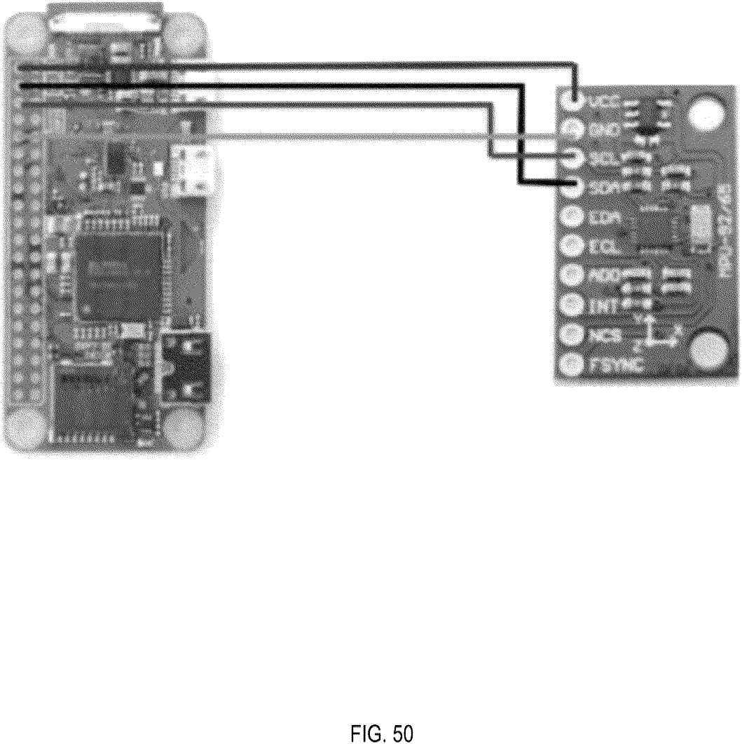

An ECG measurement was acquired from the BIOPAC system at a sampling rate of 1 kHz and used as reference. BIOPAC provides state of the art data acquisition systems and data loggers for physiological monitoring. The BIOPAC clock, which supported post-acquisition synchronization between the RPI and ECG data, was inputted to the RPI using Programmable General-Purpose Input Output (GPIO) pins. The sampling rate of the sensor setup was approximately 270 Hz. As shown in FIG. 50. GPIO pins 1 (purple), 3 (black), 5 (red), 9 (green) were used for the I2C connection to the sensor. Data acquisition was controlled by a custom built, web-based user interface and signal processing was performed using MATLAB (R2019a). The RPI acquired raw data from the IMU as well as the BIOPAC synchronization pulse and appended the data to a text file on the Micro SD card.

### FIG. 50. RPI and IMU System Configuration

### B. Data Collection

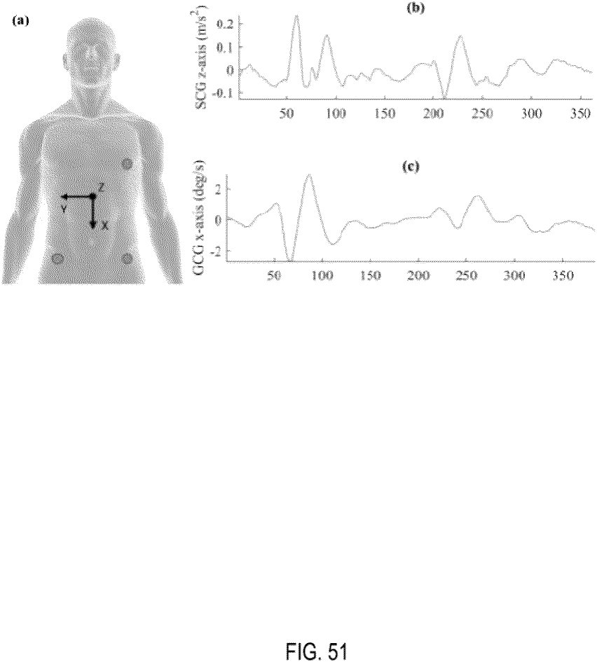

The experimental data was collected with McGill Review Ethics Board approval at McGill University. The microcontroller was strapped around the subject’s torso near the sensor. As shown in FIG. 51 (a), the X-axis, Y-axis and Z-axis of the accelerometer were oriented downward, right and outward, respectively. The gyroscope followed the righthand rule for rotation about these axes. ECG electrodes were attached to the skin in an Einthoven triangle as shown in FIG. 51 (a) and connected to the BIOPAC for measurement. A single heartbeat of the obtained signals, Z-axis acceleration and X-axis gyration, is shown in FIGS. 51 (b) and (c) respectively. The experiment contained 5 (4 Male, 1 Female) healthy subjects with no known history of cardiovascular problems. Participants were (mean): 23.6 years old, weighed 70.8 kg, with a height of 174.1 cm (Table I). After connecting the sensors, each subject performed 5 different tests. Each was a motionless experiment which lasted for a duration of 65 seconds. First was the supine position test, measured on a massage table. For the second and third tests, subjects were asked to orient themselves to face left then right. The fourth test was conducted with the participant sitting on a stool. Finally, the fifth test was a standing experiment. Tests involved relaxed breathing in the posture and orientation specified.

TABLE 7

Subjects’ Age, Weight, And Height

| Age | Weight (Kg) | Height (Cm) |
|---|---|---|
| 22 | 78.5 | 182.8 |
| 23 | 77.3 | 180.3 |
| 22 | 60 | 161.9 |
| 22 | 74.2 | 177.8 |
| 29 | 64 | 167.64 |

FIG. 51. (a) Sensor and electrode placement. (b) Z-axis acceleration. (c) Xaxis gyration.

### C. Processing

Processing included identifying R-peaks using the BIOPAC AcqKnowledge ECG annotation routines. This was followed by converting IMU raw data to acceleration/gyration values and synchronizing them with ECG using the BIOPAC clock. Autocorrelated Differential Algorithm (ADA) was used to obtain the heart rate from the mechanical signals. ADA is an SCG-based solution for real-time cardiorespiratory monitoring that employs windowing, temporal variations, and autocorrelation to yield a heart rate estimation on each evaluated second of data. It was later extended to GCG as well. Autocorrelation was selected as the foundation of the algorithm due to the quasi-periodicity of cardiac cycles and the consistency in the shape of the first heart sound. ADA was rigorously tested through physical exertion and achieved high correlation coefficients with ECG reference measurements of up to 0.97. The processing incorporated the GCG extended version of ADA to estimate heart rate.

### D. Evaluation

This paper exploits the linear relationship between ADA-derived heart rate and ECG-derived heart rate by using the Pearson’s squared correlation coefficient, r2. In order to determine trends in the data, three methods of sectioning the data were used to analyze different subsets. First, correlation coefficients were calculated on a per-test and per-subject basis, where the correlation represented the VCG-ECG relationship for a single 65 second test. It should be noted that a low amount of points leads to outliers causing significant drops in the correlation coefficient. To better analyze trends across orientation, a second subset was used where all heart rates from each test were collected across the five subjects. This produced one total r2 for each of the five orientation tests. The third subset used was to determine the change across each of the subjects. All the heart beats from one subject were collected across all the tests. This produced one total r2 for each of the five subjects. A final total correlation coefficient was shown for all the subtests and subjects combined.

### III. Results

The heart rates obtained from VCG waveforms were referenced with ECG-derived heart rates from the BIOPAC to obtain the correlation coefficients shown in Table 8. The table shows the individual correlation obtained for each subtest and subject, and when grouped together on a per-test and per-subject basis. The results show that in the de-facto SCG supine position, the algorithm produced an r2 of 0.956. When transitioning to other positions which the algorithm was not designed for, there is a small but insignificant change in correlation coefficient. The worst-case orientation was when the subject was lying on the right, with an r2 of 0.915. This demonstrates that the ADA is minimally affected by the change in orientation.

TABLE II

ADA Heart Rate Correlation Coefficients

| Test | Subject | | | | | Test Total |
|---|---|---|---|---|---|---|
| | 1 | 2 | 3 | 4 | 5 | |
| 1 | 0.942 | 0.932 | 0.765 | 0.851 | 0.808 | 0.956 |
| 2 | 0.922 | 0.867 | 0.471 | 0.669 | 0.896 | 0.915 |
| 3 | 0.943 | 0.918 | 0.595 | 0.703 | 0.762 | 0.913 |
| 4 | 0.967 | 0.900 | 0.805 | 0.910 | 0.810 | 0.943 |
| 5 | 0.961 | 0.989 | 0.762 | 0.829 | 0.918 | 0.975 |
| Subject Total | 0.994 | 0.987 | 0.935 | 0.917 | 0.915 | |

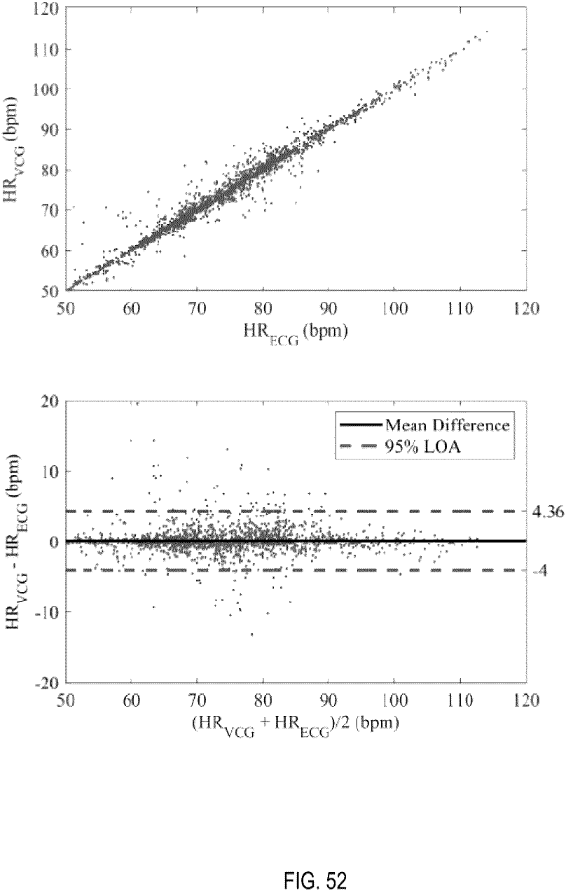

FIG. 52. Correlation and Bland Altman plots comparing VCG-derived HR to ECG-derived HR from across the entire dataset.

The Results showed that ADA successfully analyzed the VCG waveforms and obtained accurate heart rate results with a total r2 coefficient of 0.965 across the entire dataset. This is on par with the correlation produced in the literature when the algorithm was evaluated solely in the supine position. The correlation of the heart rate (HR) from VCG when referenced with ECG is shown in FIG. 52.

### IV. Discussion

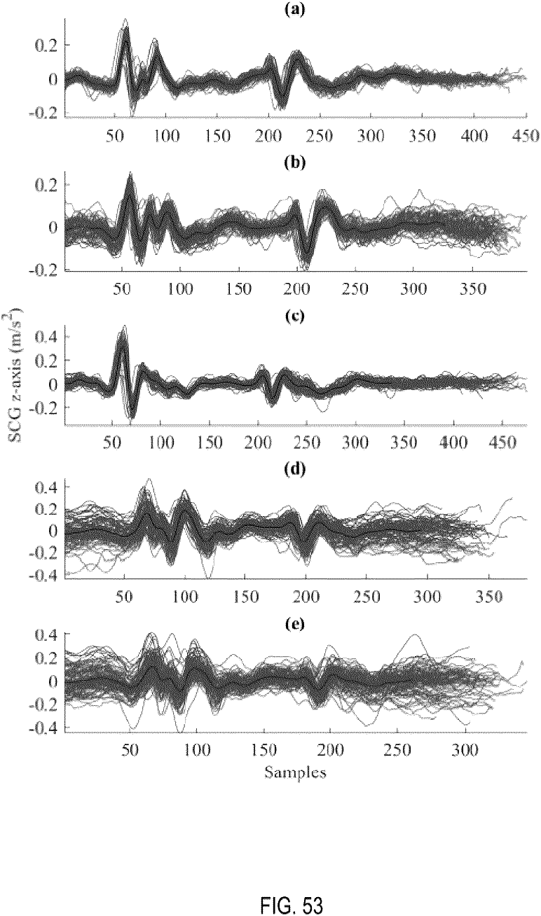

Variations in positioning of the body during heart rate monitoring introduce differences in VCG morphology. Ensemble averages in FIG. 53 were obtained by separating SCG signals from each of the 5 tests from one subject into separate cardiac cycles. The ensemble averages show variation across different orientations and postures. As expected, when transitioning from supine to upright, there is a large increase in the noise of the sensor. This can be attributed to the subject remaining more still during supine than when compared to standing. Additionally, there is a less pronounced peak within the signal. Many thresholding-based algorithms could struggle to distinguish this from the other peaks as they have a similar prominence, however, due to the lack of open source availability of SCG algorithms this was not explored quantitatively. In our results, the described ADA performed with about the same accuracy when standing versus when supine. Therefore it can be deduced that due to the feature amplification and autocorrelation, the algorithm is less sensitive to morphology changes and not constrained to the supine position.

FIG. 53. Ensemble averages for a single subject when (a) supine, (b) facing left, (c) facing right, (d) sitting, and (e) standing.

### V. Conclusion

VCG poses as a promising solution to cardiac monitoring. One of its biggest limitations is the change in morphology seen from intrapersonal effects, including orientation. We have shown that by using just a simple, wireless IMU-RPI setup, we can estimate heart rate in different orientations successfully. The tested algorithm produced a squared correlation coefficient of 0.956 when supine and 0.975 when standing, showing no significant difference. A larger study with more subjects and orientations will be conducted to prove statistical significance. These results establish a basis for further investigation into VCG morphology differences and the adaptability of detection algorithms. This study could be expanded with a second IMU to act as a differential unit to reduce motion artifact and aid in classifying the waveform.

Algorithms and techniques for detecting and determining modulation of VCG recorded via a chest worn inertial sensor due to respiration will now be described. Such algorithms and techniques may be implemented by the systems and methods for blood pressure measurement described herein, such as the systems and methods described in FIGS. 2-6. For example, the algorithms and techniques may be performed by the real-time signal processing unit 318 of the system 300 of FIG. 4 or the computer system 400 of FIG. 5 (e.g. filtering and demodulation unit 420).

Demand of portable health monitoring has been growing due to increasing cardiovascular and respiratory diseases. While both cardiovascular monitoring and respiratory monitoring have been developed independently, there lacks a simple integrated solution to monitor both simultaneously. Seismocardiography (SCG), a method of recording cardiac vibrations with an accelerometer can also be used to extract respiratory information via low frequency chest oscillations. This study used an inertial measurement unit which pairs a 3-axis accelerometer and a 3-axis gyroscope to monitor respiration while maintaining optimum placement protocol for recording SCG. Additionally, the connection between inertial measurement and both respiratory rate and volume were explored based on their correlation with a Spirometer. Respiratory volume was shown to have moderate correlation with chest motion with an average best-case correlation coefficient of 0.679 across acceleration and gyration. The techniques described will assist the design of future SCG algorithms by understanding the sources behind their modulation from respiration. This paper shows that a simplified processing technique can be added to SCG algorithms for respiration monitoring.

### I. Introduction

State-of-the-art medical care in professional settings is enabled by advanced monitoring equipment. In a clinical setting, doctors can monitor cardiovascular, respiratory, or neurological systems with ease. However, for the average person in their home, there is little access to monitoring equipment without professional assistance. At-home monitoring has been growing due to the increasing frequency of cardiovascular and respiratory diseases. Cardiovascular disease on its own represents the largest cause of death worldwide. Some of these diseases have early-onset symptoms which can mitigate the effects of cardio-respiratory diseases if detected quickly. Many individuals lack routine access to medical facilities or do not know when it is necessary to seek intervention. Portable monitoring could alleviate both issues by providing both medical professionals and users with a better understanding of their symptoms. Additionally, some cases such as atrial fibrillation, require long term monitoring as their occurrence might be dormant during medical examinations. In these examinations, most medical equipment is standardized to robust industrial applications and is generally bulky, difficult to use, or cumbersome. The prospect of wearable devices creates simplified solutions to accurately monitor health conditions without significantly interfering with daily life.

Many devices have been designed specifically for at-home monitoring. Electrocardiography represents the gold standard of cardiac monitoring. The accepted Holter monitor that is generally used for outpatient care provides a reliable estimation of cardiac information but gives no direct indication towards respiratory function. The gold standard of respiratory monitoring consists of using a mask to breathe into a spirometer. However, a mask is infeasible for measuring during daily life and activities. The most common portable method used is called Respiratory Inductive Plethysmography (RIP) which uses a deformable band across the torso to measure chest movements. Evaluations of the accuracy of RIP have reported varying results that depend on postural changes. Furthermore, existing techniques that derive breathing information from the respiratory modulation of other cardiac signals, such as electrocardiography, typically lack consistency and replicability.

Currently, there is no simple, integrated system to directly monitor cardiovascular and respiratory function simultaneously. A promising solution would be to use an inertial measurement unit (IMU). When placed on the sternum, an IMU can record cardiac vibrations in the form of accelerations, known as Seismocardiography (SCG) or in the form of gyration it is known as Gyrocardiography (GCG). During this recording, the IMU signal also contains information regarding the motion of the chest wall due to respiration. The respiratory modulation of the vibrational cardiography (VCG) signal causes changes in amplitude, baseline, and frequency – all of which reduce the accuracy of signal processing algorithms used in cardiography. It is therefore important to understand respiratory behavior when analyzing VCG signals. However, most studies provide few output parameters, a limited number of subjects, or record respiration from locations unsuitable for SCG.

In this paper, we explore the low frequency modulation of vibrational cardiography due to respiration. We extract acceleration and gyration baseline wandering to interpret the chest movements and thereby the respiratory information.

### II. Methods

### A. Data Acquisition

Data was collected with approval from the Review Ethics Board at McGill University. The study consisted of 17 (8 Female) healthy participants with no prior known cardio-respiratory ailments. The population were (mean ± standard deviation): age 23.3 ± 4.3 years, weight 67.4 ± 12.8 kg, with a height of 172 ± 9 cm.

All participants were supine and motionless for the study. Each recording lasted approximately 3 minutes in length. They were recorded in a resting state and were told to breathe normally to the best of their ability. No other instructions were given to the subject to regulate the rate or depth of their breaths.

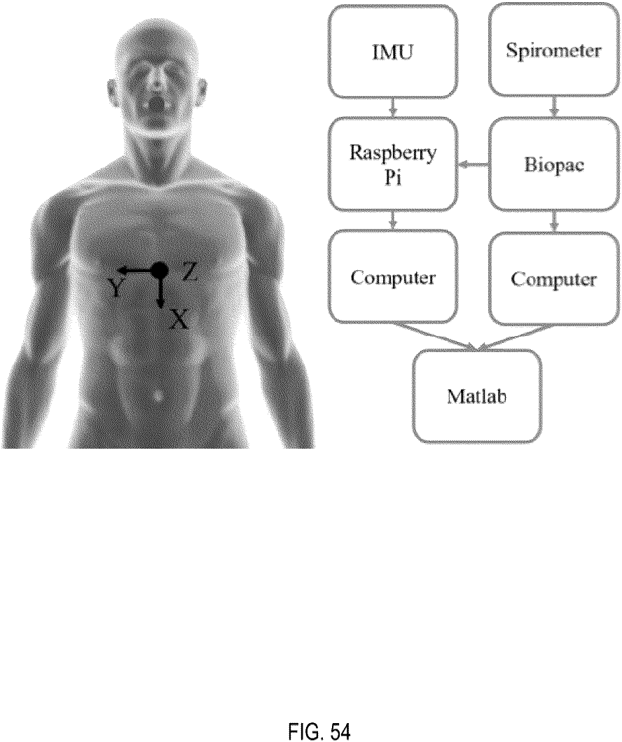

Inertial measurements were recorded by a 6 axis IMU (MPU 9250, Invensense). The device was positioned at the xiphoid process of the sternum to collect VCG recordings This positioning was used as it is the de-facto gold standard of SCG and GCG recording. There was no other optimization for respiration collection. The location is shown by the black dot in FIG. 54. A single piece of double-sided tape was used to secure the IMU to the surface of the chest. The IMU was connected to a Raspberry Pi (Pi Zero W, Raspberry) for control and data transfer. The Raspberry Pi polled the accelerometer at approximately 550 Hz and sent data through WiFi to a local computer. A digital acquisition device (MP160, Biopac) was used as reference. Airflow was monitored by a pneumotach transducer (TSD137H, Biopac) and was recorded by the Biopac system. A 3 L syringe was used before the test to calibrate the volume generated by the spirometer flow measurement. A clock signal was generated by the Biopac and connected to the Pi to synchronize the two systems.

FIG. 54. a) Spirometer (red) and IMU (black) placement with corresponding acceleration coordinates. b) Experimental dataflow diagram.

### B. Methods and Analysis

After the recording, all data was processed in Matlab (R2019A). Air flow from the mouth was recorded by the spirometer while the nose was clamped. The flow was smoothed and integrated to measure respiratory volume. The volume was calibrated to a 3 L syringe before each test. The drift caused by numerical integration was removed by fitting a 2ndorder polynomial to the recording and subtracting it. Removal of the polynomial offset produced a stable respiratory volume.

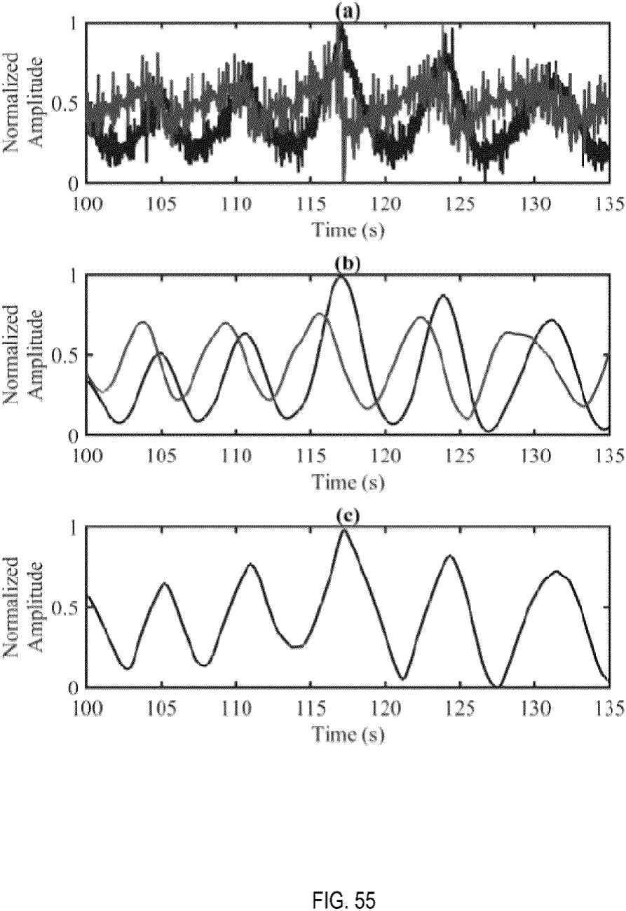

The 6-axis IMU data was interpolated to 200 Hz to match the required sampling rate needed for SCG. Given the orientation of the sensor, the strongest and most periodic cardiac vibrations were generally found in the az, gx, and gy axes. Respiration can often be found, at least partially, in all 6 axes. The strongest consistent respiration was found in the ax and gy axes, which can be seen in FIG. 55(a). A 4thorder Savitsky-Golay filter was used to remove both high-frequency noise and cardiovascular vibrations. A Savitsky-Golay filter was chosen due to its efficiency when removing noise from a wide frequency range. The filter incorporated a variable window size according to respiration frequency. This was determined by the frequency domain across all 6 axes. All spectra were normalized and summed within the range of 0-2 Hz. The maximum frequency from the resulting single spectrum was assumed to be the respiration frequency. The frame length of the filter was adjusted to be proportional to the size of the respiration period. Due to the large random spikes from cardiac vibrations, particularly in the gy axis, the two filtered signals were smoothed by a moving average filter with a window size of 0.75 seconds. An example of the final estimated waveform can be seen in FIG. 55(b).

FIG. 55. a) Raw x-axis acceleration (red) and y-axis gyration (blue), (b) Savitsky-Golay filtered x-axis acceleration (red) and y-axis gyration (blue), (c) reference lung volume. All plots were normalized.

To quantitatively compare the estimated waveform with the reference, the peak inhalation volumes were annotated along with the corresponding positive oriented peak in the acceleration or gyration domains. All datasets were manually inspected such that the same number of breaths were used for correlation purposes.

### III. Results

We analyzed 582 breaths across 17 subjects. Pairs of observed peak volumes and estimated peak volumes (via acceleration and gyration) were analyzed for both respiration rate and volume. The metric used to determine their linear dependency was the Pearson’s correlation coefficient.

First, the relationship between the IMU sensor and respiration rate was evaluated. Respiration rate is used as a common indicator towards health status and obstructive diseases. Respiratory rate is a primary indicator when evaluating respirational function and therefore it was included as a preliminary metric. Across all subjects, the combined respiration rate resulted in a correlation coefficient of 0.895 for acceleration and 0.828 for gyration. Note that no additional processing was done on the filtered signals. This high correlation confirms their ability to detect respiratory motion and has potential to be used in a more refined algorithm for portable devices.

The second metric evaluated was respiration volume. Generally, the methods to extract respiration volume are more difficult and often require additional calibration or produce unstable results. We analyzed the relationship between the relative changes in volume amplitude and the relative corresponding acceleration or gyration values. The results for each test are summarized in Table 9. In most subjects, the accelerometer had a better correlation than the gyroscope. However, in five of the 17 tests, the gyroscope had a better correlation. This could be manipulated by a decision-making algorithm to select either the acceleration or gyration-derived result. In this work, the final column in Table 9 shows maximum result from the two methods.

TABLE 9

Correlation Results of Acceleration, Gyration, and Combined Best Case

| File | Acceleration | Gyration | Max |
|---|---|---|---|
| 1 | 0.817 | 0.474 | 0.817 |
| 2 | 0.813 | 0.516 | 0.813 |
| 3 | 0.752 | 0.226 | 0.752 |
| 4 | 0.738 | 0.322 | 0.738 |
| 5 | 0.681 | 0.441 | 0.681 |
| 6 | 0.675 | -0.158 | 0.675 |
| 7 | 0.675 | 0.045 | 0.675 |
| 8 | 0.629 | -0.137 | 0.629 |
| 9 | 0.531 | 0.101 | 0.531 |
| 10 | 0.121 | 0.725 | 0.725 |
| 11 | -0.037 | 0.655 | 0.655 |
| 12 | 0.316 | 0.592 | 0.592 |
| 13* | 0.862 | 0.571 | 0.862 |
| 14* | 0.630 | -0.877 | 0.630 |
| 15* | 0.584 | -0.107 | 0.584 |
| 16* | 0.436 | 0.661 | 0.661 |
| 17* | -0.184 | 0.515 | 0.515 |
| Average | 0.532 | 0.269 | 0.679 |

*Indicates Inverted Signals

Additionally in five subjects, the corresponding peaks were inverted from the reference signal. This is likely due to variations in sensor placement, body morphology, or breathing patterns of the subject. In these tests, the estimated waveform was inverted and then the local maximums were correlated to the respiration value. Across all 17 subjects, these methods resulted in an average correlation of 0.532 for acceleration, 0.269 for gyration, and 0.679 when considering the best of each result.

### IV. Discussion

There are three main metrics to consider when understanding respiration: rate, volume and phase. Using an accelerometer and gyroscope proved to be sufficient for measuring respiration rate, as expected from the literature. While this study shows that there is a relationship between respiration volume and chest movement, it needs a more refined algorithm to extract reliable data. This could be accomplished with some type of calibration between the sensor, placement and breathing patterns of the subject. Also, there is currently no automatic way to determine which is the best axis to use for estimation and therefore a fusion algorithm should be considered for a real-world implementation.

Only two axes were considered whereas all 6 could potentially be indicative of respiratory volume. These orientations could be included in detection/rejection or fusing algorithms. Although phase was initially considered in this study, there was no clear and obvious indicator towards respiration phase using these methods. Each test appeared to have a shift in phase between the reference and each of the six axes, the in-phase value differing on a per-subject basis. It is possible as well that given a more advanced algorithm, an inertial sensor could be able to predict respiratory phase.

This study is limited to a controlled environment where subjects were constrained to a motionless and supine setting. In a real-world scenario, additional filtering and processing would be required to remove motion artifact from the signal. The study is limited as it only considered healthy subjects with normal breathing. An extension would be to include varying breathing patterns, rates, and depths to get a better understanding to how the baseline wandering is affected by tidal volume.

### V. Conclusion

An IMU was placed on the xiphoid process of the sternum. From this placement, cardiac and respiratory information are recorded with a single sensor. While this location has been heavily characterized for VCG, there is more to be done for respiration. This paper showed that without much filtering and processing - using only a Savitsky-Golay filter, that respiration rate and respiration volume can be detected in a controlled and supine environment. Respiration rate was detected with a correlation coefficient of 0.895. Respiration volume had a weaker relationship with a correlation coefficient of 0.679. A deeper exploration concluded there were inconsistencies regarding the optimal volume detection between the accelerometer and the gyroscope. Therefore a respiratory detection algorithm should use a fusion of the two sensors to increase accuracy performance.

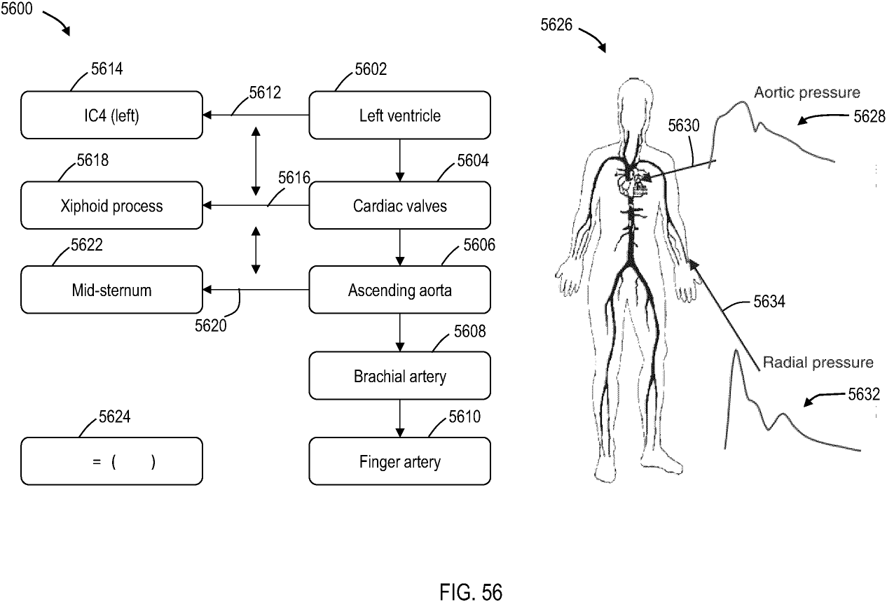

Referring now to FIG. 56, shown therein is a schematic representation 5600 of cardiac system blood flow from the left ventricle to the finger artery and corresponding vibrational activity associated with cardiac mechanical activity of the blood flow, which can be leveraged by the systems and methods for hemodynamic measurement of the present disclosure.

The cardiac system blood flow moves from the left ventricle 5602 to the cardiac valves 5604, to the ascending aorta 5606, to the brachial artery 5608, to the finger artery 5610. Blood pressure can thus be measured at the finger artery 5610 and used as a point of comparison for the effectiveness of the systems and methods for blood pressure determination described herein.

Vibrations 5612 resulting from cardiac mechanical activity at the left ventricle 5602 are sensed most strongly around left intercostal (IC) 4 5614. Vibrations 5616 resulting from motion of the cardiac valves 5604 are sensed most strongly at the xiphoid process 5618. The xiphoid process 5618 also represents a most stable placement point for the vibration sensor. Vibrations 5620 resulting from motion of the ascending aorta 5606 can be sensed at the mid-sternum 5622.

The relationship identified and established in the present disclosure and used in the systems and methods described herein between blood pressure estimate and vibrations detected at the surface of the chest through VCG data (vibration signal) is shown at 5624.

FIG. 56 further illustrates a schematic representation 5626 of an aortic pressure waveform 5628 corresponding to blood pressure at the aorta 5630 and a radial pressure waveform 5632 corresponding to blood pressure at the radial artery 5634.

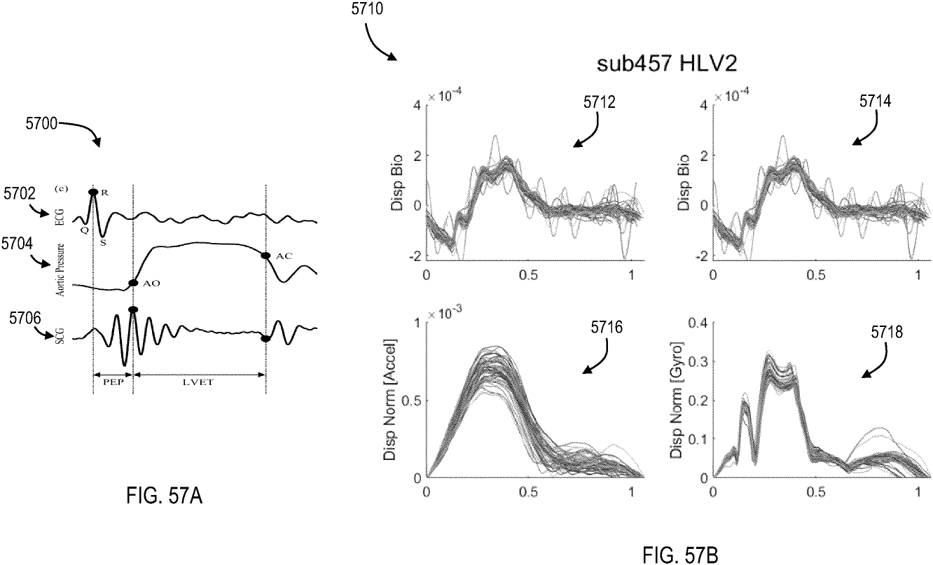

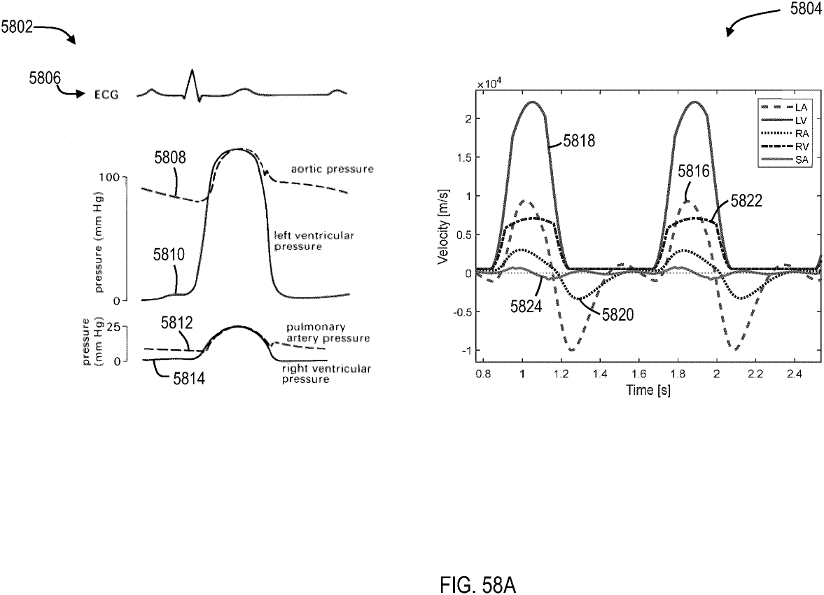

FIGS. 57A and 57B will now be described.

FIG. 57A is a graphical representation 5700 of an ECG waveform 5702, aortic pressure waveform 5704, and SCG waveform 5706 over time including a pre-ejection period (PEP) and left ventricular ejection time (LVET). The SCG waveform 5706 corresponds to a vibration signal sensed at the surface of the chest, as described herein.

FIG. 57B is a graphical representation 5710 showing curves of linear displacement 5712, 5716 and angular displacement 5714, 5718 for the purposes of illustrating the relationship between the vibration signal (i.e. displacement) and cardiac pressure differentials. Curves 5712 and 5714 are generated by integrating the motion signal of the SCG 5706 twice and once, respectively (i.e. double and single integration). Curve 5716 is a vector norm taken of three axes for linear displacement. Curve 5718 is a vector norm taken of three axes for angular displacement. The vector norm was used to track the displacement magnitude as a way to combine all three axes using the root mean square.

In particular, the displacement curve 5716 illustrates that the vibration signal (SCG signal) being sensed in the systems and methods of the present disclosure is related to cardiac pressure differentials.

FIG. 57A also shows a coincidence between the rise and fall of aortic pressure 5704 with the occurrence of vibrational pulses V1 and V2 in the SCG signal 5706, which are marked as the aortic opening (AO) and aortic closure (AC), respectively, indicating the systolic phase of the cardiac cycle. The pressure waveform in an artery is directly related to the volumetric expansion of the artery to accommodate the increase in pressure. This expansion can be measured as the increase in diameter of the artery, or the outward displacement of a motion sensor attached to the artery. Hence, the aortic pressure waveform 5704 is related to the displacement signals observed in the motion sensor as shown in FIG. 57B.

From this, we know that the pressure waveform in an artery is directly related to the volumetric expansion of the artery. This expansion can be measured as the increase in diameter of the artery, or the outward displacement of a motion sensor attached to the artery. A similar principle is used by a finger cuff when measuring blood pressure. The present disclosure thus provides that the displacement therefore provides an indication of central aortic pressure.

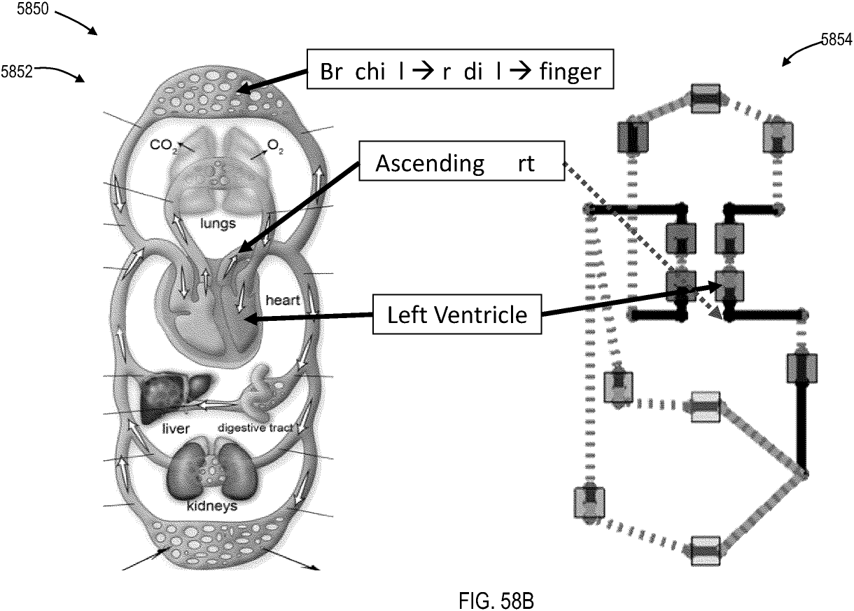

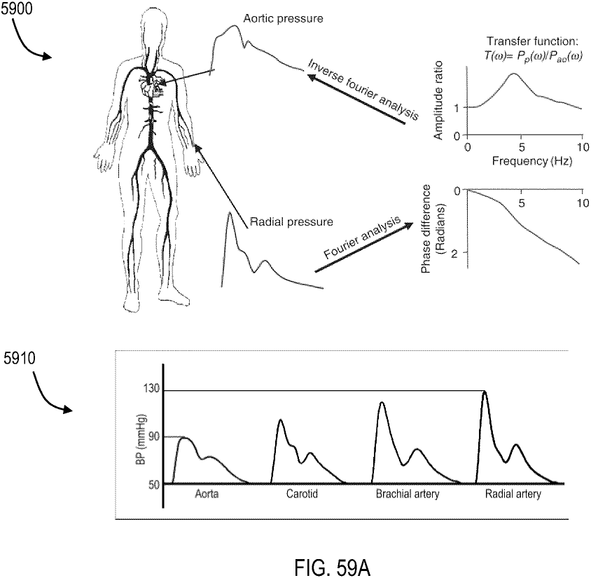

FIGS. 58A and 58B will now be described.

FIG. 58A includes graphs 5802 and 5804. FIG. 58B shows a graphical representation 5850 including a cardiac system representation 5852 and a cardiac model 5854 of the cardiac system achieved mechanically and used to prove connection between vibrations and cardiac pressure, and the relationship between the cardiac system representation 5852 and the cardiac model 5854. The cardiac model 5854 is a mechanical analog to the fluidics in the heart that was developed so that the flow of blood could be modelled using a mass-spring-damper system.

Graph 5802 shows an ECG waveform 5806, and pressure waveforms for aortic pressure 5808, left ventricular pressure 5810, pulmonary artery pressure 5812, and right ventricular pressure 5814.

Graph 5804 illustrates velocity curves over time for the left atrium 5816, left ventricle 5818, right atrium 5820, right ventricle 5822, and sinoatrial node 5824. The velocity is a representation of or proxy for pressure.

Graph 5804 of FIG. 58A is an output of the cardiac model 5854 whose schematic is shown in 58B. Graph 5802 includes curves 5806-5814, which represent what the system is trying to model using model 5854. In FIG. 58B, the model 5854 is a mass spring damper system that represents certain parts of the circulatory system 5852. Certain parts of interest of the circulatory system 5852 which are modelled by the model 5854 are indicated in FIG. 58B by the labelled arrows. In graph 5804, the velocity represents pressure, and acceleration represents dP/dt.

As can be seen, graph 5804 is remarkably similar to a conventional Wiggers diagram, such as shown in FIGS. 12 and 27. In this sense, the graph 5804 may be considered the Wiggers diagram of the model 5854. Graph 5804 (and model 5854) is not measuring pressure in the aorta, but rather measuring voltage and currents that are computed in the model. While the measurements produced by the model 5854 (e.g. in graph 5804) are not the same as in graph 5802, the relationship and the curves are similar, providing a basis to confirm that the modelling performed using the model 5854 is pertinent. Once calibrated, this can allow describing of the pressure flux in the aorta and, potentially, anywhere else in the system. Calibration may be performed, for example, using catheterized measurements in the aorta. The model 5854 in FIG. 58B includes boxes and dashes which show fluid flow and pressures at various points in the body (extremities and abdomen not done). From here, it can be seen how measuring vibration at a central point on the body (e.g. xiphoid process, surface of the chest) can enable the measurement or inference of pressures in vital organs using the systems and methods of the present disclosure.

In graph 5804, velocity is a proxy for pressure. It is the velocity of the displacement or the dt of the aorta swelling and contracting as blood injecting into it. The vibration being monitored is an effect of blood being pushed out of the ventricle with some force and with some impact into the aorta. The aorta bulges with pulsatile flow, the left ventricle collapses, and the aortic valve opens. Blood then fills aorta, which causes a bulging. The bulging and returning represents a primary source of vibration which the systems and methods of the present disclosure are configured to sense and measure at the surface of the chest (xiphoid process).

The work performed through the cardiac model 5854 and the outputs (e.g. 5804) therefrom indicates a connection between displacement and vibration; in particular, that vibration is caused by displacement and displacement is caused by the pressure pulse, which is caused by contraction of the heart. Thus, the present disclosure provides that the vibrations sensed and recorded by the systems and methods herein are characteristic of the pressure wave (as shown, for example, by the correspondence between the curves of graphs 5802 and 5804) and the vibration signals being measured can be used to estimate blood pressure because of the demonstrated connection between displacement and vibration.

FIG. 58B shows a graphical representation 5850 including a cardiac system representation 5852 and a cardiac model 5854 of the cardiac system achieved mechanically and used to prove connection between vibrations and cardiac pressure,and the relationship between the cardiac system representation 5852 and the cardiac model 5854.

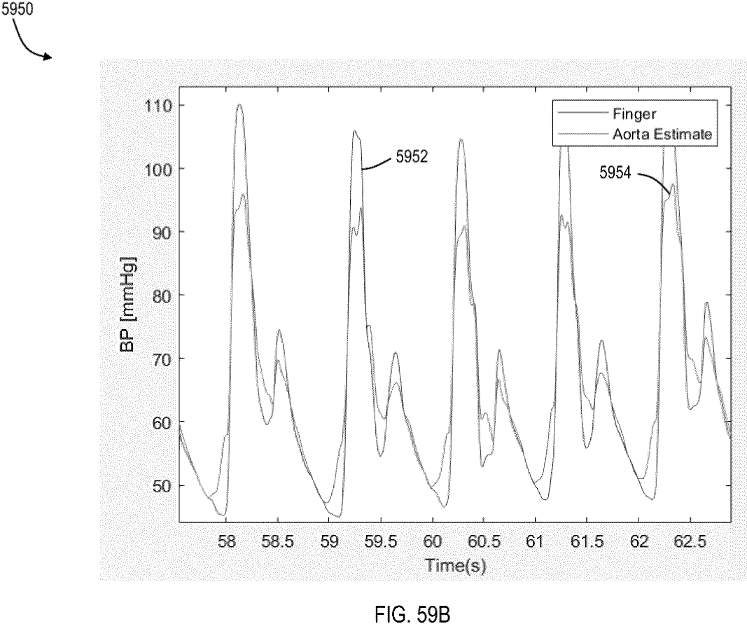

FIGS. 59A and 59B will now be described.

FIG. 59A is a graphical representation 5900 of a transfer function associated with cardiac pressure change and a graph 5910 illustrating evolution of the blood pressure waveform from aorta to finger (aorta, carotid artery, brachial artery, radial artery).

When blood is pumped from the heart, the blood pressure waveform has a certain morphology, or shape. As the pressure pulse travels along the arterial tree, it undergoes branching, reflections, and modulation, which change its morphology. The blood pressure waveform at the finger is quite different from that at the heart although they have certain similar characteristics. The transfer function essentially models (e.g. through manipulations to data) the change of the waveform from aorta to radial. It is also shown in 5910.

FIG. 59B is a graph 5950 showing blood pressure curves over time for finger measurement 5952 and an aorta estimate 5954. The graph 5950 shows the application of the transfer function to try and reproduce a waveform similar to what is expected for the aorta waveform.

All three graphs in FIGS. 58A, 58B show the evolution of the pressure waveform from the aorta to an extremity for a single cardiac cycle. In FIG. 59A, this is the radial artery while in FIG. 59B, this is the finger artery. The aortic pressure waveform in all three graphs is supposed to be the same.

While the above description provides examples of one or more apparatus, methods, or systems, it will be appreciated that other apparatus, methods, or systems may be within the scope of the claims as interpreted by one of skill in the art.

1-12. (canceled)

13. A system for non-invasive blood pressure measurement of a subject, the system comprising:
a sensor device including an accelerometer and a gyroscope, the sensor device for detecting vibrations at the surface of the chest of the subject corresponding to cardiac mechanical activity of the heart and transmitting a vibration signal associated with the detected vibrations;
a computing device communicatively connected to the sensor device via a data communication link, the computing device including:
a communication interface for receiving the vibration signal from the sensor device via the data communication link;
a processor configured to determine a vibration feature from the vibration signal, determine a blood pressure measurement from the vibration feature, and generate a human-readable format of the blood pressure measurement;
a memory for storing the blood pressure measurement; and
a display device for outputting the blood pressure measurement in the human-readable format.

14. The system of claim 13, wherein the processor is further configured to identify vibrational pulses V1 and V2 from vibrational cardiography (VCG) data, the VCG data derived from the vibration signal, and determine the vibration feature from the vibrational pulses V1 and V2.

15-16. (canceled)

17. The system of claim 13, wherein determining the blood pressure measurement by the processor includes determining maxima, minima, or mean of a central aortic or left ventricular pressure waveform for each cardiac cycle in real-time.

18. The system of claim 13, wherein the vibration signal includes a linear acceleration component and a rotational velocity component.

19. The system of claim 18, wherein the vibration signal includes six orthogonal motion signals.

20. The system of claim 13, wherein determining the vibration feature by the processor includes quantifying the fraction of energy of stroke volume converted to vibration.

21. The system of claim 13, wherein determining the vibration feature by the processor includes determining any one or more of jerk, amplitude, frequency, phase, and a cardiac time interval from a linear acceleration component or rotational velocity component of the vibration signal.

22-23. (canceled)

24. A computer system for non-invasive blood pressure measurement of a subject, the system comprising:
a communication interface for receiving a vibration signal, the vibration signal detected at the surface of the chest of the subject and corresponding to cardiac mechanical activity of the heart;
a processor configured to:
generate vibrational cardiography (VCG) waveform data from the vibration signal;
filter and demodulate the VCG waveform data to generate a processed VCG waveform;
determine a vibrational feature from the processed VCG waveform data;
determine a blood pressure measurement from the vibrational feature;
generate a human-readable format of the blood pressure measurement;
a display device for outputting the blood pressure measurement in the human-readable format.

25. The system of claim 24, wherein the processor is further configured to identify vibrational pulses V1 and V2 from the processed vibrational cardiography waveform data and determine the vibration feature from the vibrational pulses V1 and V2.

26-27. (canceled)

28. The system of claim 24, wherein determining the blood pressure measurement from the vibrational feature by the processor includes determining maxima, minima, or mean of a central aortic or left ventricular pressure waveform for each cardiac cycle in real-time.

29. The system of claim 24, wherein the vibration signal includes a linear acceleration component and a rotational velocity component.

30. The system of claim 29, wherein the vibration signal includes six orthogonal motion signals.

31. The system of claim 24, wherein determining the vibration feature from the processed VCG waveform data by the processor includes quantifying the fraction of energy of stroke volume converted to vibration.

32. The system of claim 24, wherein determining the vibration feature from the processed VCG waveform data by the processor includes determining any one or more of jerk, amplitude, frequency, phase, and a cardiac time interval from a linear acceleration component or rotational velocity component of the vibration signal.

33-34. (canceled)

35. A method of non-invasive hemodynamic measurement of a subject, the method comprising:
identifying vibrational pulses V1 and V2 from vibrational cardiography (VCG) data, the VCG data derived from a vibration signal acquired at the surface of the chest of the subject corresponding to cardiac-induced vibrations;
determining a vibration feature from the vibrational pulses V1 and V2; and
determining a hemodynamic measurement from the vibration feature.

36-42. (canceled)

43. The method of claim 35, wherein determining the hemodynamic measurement includes determining blood pressure measurement, and wherein determining blood pressure measurement includes determining maxima, minima, or mean of a central aortic or left ventricular pressure waveform for each cardiac cycle in real-time.

44. The method of claim 35, wherein the vibration signal includes six orthogonal motion signals.

45. The method of claim 37, wherein determining the vibration feature includes quantifying the fraction of energy of stroke volume converted to vibration.

46. The method of claim 35, wherein the vibration feature is determined using a linear acceleration component of the vibration signal and a rotational velocity component of the vibration signal.

47. The method of claim 35, wherein determining the vibration feature includes determining any one or more of jerk, amplitude, frequency, phase, and a cardiac time interval from a linear acceleration component or rotational velocity component of the vibration signal.
